(19) United States
(12) Patent Application Publication
(10) Pub. No.: US 2023/0174669 A1
(43) Pub. Date: Jun. 8, 2023

(54) Anti-CD98 Antibodies And Uses Thereof

(71) Applicant: Aliada Therapeutics, Inc., Boston, MA (US)

(72) Inventors: Suzanne Edavettal, San Diego, CA (US); Sanjaya Singh, Spring House, PA (US); Derrick Domingo, San Diego, CA (US); Deepti Wilkinson, Del Mar, CA (US); Pilar Cejudo-Martin, San Diego, CA (US); Pharavee Jaiprasart, Spring House, PA (US); Brian Geist, Spring House, PA (US)

(21) Appl. No.: 17/995,766

(22) PCT Filed: Apr. 7, 2021
(86) PCT No.: PCT/IB2021/052892

§ 371 Date: Oct. 7, 2022

Related U.S. Application Data
(60) Provisional application No. 63/006,988, filed on Apr. 8, 2020.
(60) Provisional application No. 63/035,961, filed on Jun. 8, 2020.

(51) Int. Cl. C07K 16/28; A61K 47/68; C07K 16/18; A61P 25/28; C07K 16/40

(52) U.S. Cl. CPC C07K 16/2896; A61K 47/6849; C07K 16/18; A61P 25/28; C07K 16/40; A61K 2039/505

(57) ABSTRACT

Monoclonal anti-CD98 antibodies and antigen-binding fragments thereof for delivering an agent to the brain of a subject in need thereof are described. Also described are conjugates and fusion constructs containing the anti-CD98 antibody or antigen-binding fragment thereof coupled to a therapeutic or diagnostic agent, such as a second antibody and antigen-binding fragment thereof, for treating or detecting a neurological disorder and/or delivering a therapeutic or diagnostic agent across the blood-brain barrier. Also described are nucleic acids encoding the antibodies, conjugates and fusion constructs and related recombinant host cells.

### CROSS REFERENCE TO RELATED APPLICATION

This application claims priority to U.S. Provisional Application No. 63/006,988, filed on Apr. 8, 2020, and U.S. Provisional Application No. 63/035,961, filed on Jun. 8, 2020, the disclosures of which are incorporated herein by reference in their entireties.

### REFERENCE TO SEQUENCE LISTING SUBMITTED ELECTRONICALLY

This application contains a sequence listing, which is submitted electronically via EFS-Web as an ASCII formatted sequence listing with a file name “JBI6290WOPCT1_Sequence_Listing” and a creation date of Mar. 31, 2021, and having a size of 478 kB. The sequence listing submitted via EFS-Web is part of the specification and is herein incorporated by reference in its entirety.

### FIELD OF THE INVENTION

The present invention relates to a blood-brain barrier shuttle that binds to human CD98hc, and methods of using the same.

### BACKGROUND OF THE INVENTION

While the blood-brain barrier (BBB) prevents harmful substances from entering the brain and is essential for brain homeostasis, it presents a formidable obstacle for efficiently delivering drugs to the brain. Large molecules, such as monoclonal antibodies and other biotherapeutics, have great therapeutic/diagnostic potential for treating/detecting pathology in the central nervous system (CNS). However, their route into the brain is prevented by the BBB.

Previous studies have illustrated that only a very small percentage (approximately 0.1%) of an IgG injected in the bloodstream are able to penetrate the BBB into the CNS compartment (Felgenhauer, Klin. Wschr. 52: 1158-1164, 1974)). This will limit any pharmacological effect due to the low concentration within the CNS of the antibody.

Numerous approaches have been studied to improve the brain delivery of therapeutic monoclonal antibodies (mAbs), including the use of receptor-mediated transcytosis (RMT). RMT utilizes abundantly expressed receptors on the luminal side of the BBB for transport through brain endothelial cells. Previous efforts to generate a clinically feasible platform for delivery of therapeutic mAbs into the brain have been focused on antibody engineering to increase the efficiency of transcytosis, with gains made through observations on valency of binding, pH dependency and affinity (reviewed in Goulatis et al., 2017, Curr Opin Struct Biol 45: 109-115). However, translation into non-human primates (NHPs) and the clinic has been limited by rapid peripheral clearance from target-mediated drug disposition (TMDD) and safety from acute reticulocyte depletion (Gadkar K, et al. Eur J Pharm Biopharm. 2016; 101: 53-61).

CD98 heavy chain subunit (CD98hc) is a member of the solute carrier family and heterodimerizes with a number of CD98 light chain members to form amino acid transporters at the BBB (Zuchero Y J, et al. Neuron. 2016; 89(1): 70-82, citing Boado R J, et al. PNAS 1999; 96(21): 12079-84). The intracellular portion of CD98hc functions to mediate integrin signaling, which plays a role in both cell growth and tumorigenesis (Zuchero Y J, et al. Neuron. 2016; 89(1): 70-82, citing Feral C C, et al. J Cell Biol 2007; 178: 701-711; Cantor J M and Ginsburg M H. J Cell Sci 2012; 125: 1373-82). CD98hc is highly expressed on human brain microvasculature, and anti-CD98hc bispecific antibodies have been used to deliver a therapeutic antibody to the mouse brain (Zuchero Y J, et al. Neuron. 2016; 89(1): 70-82). However, the ability to target CD98hc safely in human has yet to be investigated.

Therefore, there is a need for an anti-CD98 monoclonal antibody or antigen binding fragment thereof that can be used to shuttle drugs into the brain efficiently with improved safety and PK.

### SUMMARY OF THE INVENTION

The application relates to an optimized platform for brain delivery, accounting for not just brain concentration of a delivered agent, such as a therapeutic monoclonal antibody (mAb), but also therapeutically relevant characteristics of the mAb, including peripheral pharmacokinetics, safety and the pharmacodynamics of the mAb. The platform utilizes a CD98 binding molecule, in particular, an antibody or antigen-binding fragment thereof that binds to CD98, preferably a human CD98 heavy chain (huCD98hc), wherein the CD98 binding molecule has optimized transport function defined by the on-rate kₐ and off-rate k_{d} values both at a neutral pH of 6.8 to 7.8, such as a physiological pH (e.g., 7.4), and at an acidic pH of 4.5 to 6.5, such as an acidic pH often found in endosomal compartments.

The inventors discovered, unexpectedly, the optimal values are not simply the fastest on-rate kₐ values and the slowest off-rate k_{d} values as one might expect in typical antibody-target interactions. That is, for this system, one would not necessarily want to use a molecule that “binds” and associates with CD98 at a relatively high rate and then dissociates from the CD98 more slowly to have the longest life span of the antibody-target complex. Instead, in one embodiment, the optimized transport function of the CD98 binders described herein preferably have kₐ rates that are similar (e.g., within the same order of magnitude) at both physiologic pH (e.g., 7.4) and at lower pH (e.g., 6.5 or 6.0) but have faster off-rate k_{d} at a lower pH (e.g., pH 6.5 or 6.0) when compared to the k_{d} rates at physiological pH (e.g., 7.4).

In one general aspect, the application describes an anti-CD98 antibody or antigen-binding fragment thereof for delivering a therapeutic or diagnostic agent to the brain of a subject in need thereof, wherein the anti-CD98 antibody or antigen-binding fragment thereof binds to a CD98, preferably human CD98hc, with a dissociation constant K_{D} of at least 1 nM, preferably 1 nM to 500 nM at neutral pH and an off-rate constant k_{d} of at least 10⁻⁴ sec⁻¹, preferably 10⁻⁴ to 10⁻¹ sec⁻¹, at an acidic pH, preferably pH 5.

In some embodiments, an anti-CD98 antibody or antigen-binding fragment thereof of of the application has an off-rate constant k_{d} of 2×10⁻² to 2×10⁻⁴ sec⁻¹, preferably 9×10⁻³ sec⁻¹ at a neutral pH.

In another embodiment, the optimized transport function of certain CD98 binders described herein preferably have a kₐ rate of at least 1.05×10⁵ and a k_{d} rate of at least 9×10⁻³ s⁻¹ or faster at physiologic acidic pH (e.g., 7.4). The aforementioned pH, KD, kₐ and k_{d} parameters reflect optimized transcytosis conditions only and in no way limit our findings that CD98-mediated transport, of certain molecules conjugated to certain CD98 binders herein, may nonetheless occur outside of the preferred parameters described.

In one general aspect, the application relates to an antibody or antigen-binding fragment thereof for delivering an agent to the brain of a subject in need thereof, wherein the antibody or antigen-binding fragment thereof binds to CD98, preferably a human CD98 heavy chain (huCD98hc), comprising
(1) a heavy chain variable region comprising heavy chain complementarity determining regions (HCDRs) HCDR1, HCDR2 and HCDR3, and a light chain variable region comprising light chain complementarity determining regions (LCDRs) LCDR1, LCDR2 and LCDR3, wherein the HCDR1, HCDR2, HCDR3, LCDR1, LCDR2 and LCDR3 have the amino acid sequences of:
   (i) SEQ ID NOs: 7, 8, 9, 10, 11 and 12, respectively;
   (ii) SEQ ID NOs: 67, 68, 69, 70, 71 and 72, respectively;
   (iii) SEQ ID NOs: 89, 90, 91, 92, 93 and 94, respectively;
   (iv) SEQ ID NOs: 113, 114, 115, 116, 117 and 118, respectively;
   (v) SEQ ID NOs: 123, 124, 125, 126, 127 and 128, respectively;
   (vi) SEQ ID NOs: 133, 134, 135, 136, 137 and 138, respectively;
   (vii) SEQ ID NOs: 150, 151, 152, 153, 154 and 155, respectively;
   (viii) SEQ ID NOs: 160, 161, 162, 163, 164 and 165, respectively;
   (ix) SEQ ID NOs: 170, 171, 172, 173, 174 and 175, respectively;
   (x) SEQ ID NOs: 180, 181, 182, 183, 184 and 185, respectively;
   (xi) SEQ ID NOs: 194, 195, 196, 197, 198 and 199, respectively; or
   (xii) SEQ ID NOs: 204, 205, 206, 207, 208 and 209, respectively; or
(2) a single variable domain on a heavy chain (VHH) comprising heavy chain complementarity determining regions (HCDRs) HCDR1, HCDR2 and HCDR3 having the amino acid sequences of:
   (i) SEQ ID NOs: 29, 30 and 31, respectively;
   (ii) SEQ ID NOs: 48, 49 and 50, respectively; or
   (iii) SEQ ID NOs: 143, 144 and 145, respectively.

In certain embodiments, the application relates to an anti-CD98 VHH fragment comprising an amino acid sequence having at least 80%, such as at least 85%, 90%, 95% or 100%, sequence identity to SEQ ID NO: 28, SEQ ID NO: 47, or SEQ ID NO: 142.

In other embodiments, the application relates to an anti-CD98 single-chain variable fragment (scFv) comprising a heavy chain variable region covalently linked to a light chain variable region via a linker, preferably, the linker has the amino acid sequence of SEQ ID NO: 189. More preferably, the scFv comprises an amino acid sequence having at least 80%, such as at least 85%, 90%, 95% or 100%, sequence identity to the amino acid sequences of SEQ ID NO: 6, SEQ ID NO: 66, SEQ ID NO: 88, SEQ ID NO: 112, SEQ ID NO: 122, SEQ ID NO: 132, SEQ ID NO: 149, SEQ ID NO: 159, SEQ ID NO: 169, SEQ ID NO: 179, SEQ ID NO: 193, or SEQ ID NO: 203.

Another aspect of the application relates to a conjugate comprising an anti-CD98 antibody or antigen-binding fragment thereof of the application coupled to a therapeutic or diagnostic agent, such as a neurological disorder drug or an agent for detecting a neurological disorder. Preferably, the therapeutic or diagnostic agent is a second antibody or an antigen binding fragment thereof that binds to a brain target.

In certain embodiments, the application relates to a fusion construct comprising an anti-CD98 antibody or antigen-binding fragment thereof of the application covalently linked to a second antibody or an antigen binding fragment thereof that binds to a brain target, such as a brain target selected from the group consisting of beta-secretase 1 (BACE1), amyloid beta (Abeta), epidermal growth factor receptor (EGFR), human epidermal growth factor receptor 2 (HER2), Tau, apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), and caspase 6.

In certain embodiments, a fusion construct of the application comprises a second antibody or antigen binding fragment thereof that binds to Tau and comprises HCDR1, HCDR2, HCDR3, LCDR1, LCDR2 and LCDR3 having the amino acid sequences of SEQ ID NOs: 213-218, respectively. Preferably, the second antibody is a monoclonal antibody comprising a heavy chain having the amino acid sequence of SEQ ID NO: 110 and a light chain having the amino acid sequence of SEQ ID NO: 111.

In one embodiment, a fusion construct of the application comprises an anti-CD98 antibody or antigen-binding fragment thereof, preferably an anti-huCD98hc VHH or scFv fragment, of the application covalently linked, via a linker, to the carboxyl terminus of only one of the two heavy chains of a second antibody or antigen binding fragment thereof that binds to a brain target. Preferably, the linker has the amino acid sequence of SEQ ID NO: 190 or SEQ ID NO: 191.

In certain embodiments, each of the two heavy chains of the second antibody or antigen binding fragment thereof comprises a modified constant heavy chain 3 (CH3) domain as compared to a wild-type CH3 domain to facilitate the formation of a heterodimer between the two heavy chains. Any mutation that facilitates the formation of a heterodimer between the two heavy chains can be used. Preferably, the modified CH3 domain of the first heavy chain comprises amino acid modifications at positions T350, L351, F405, and Y407, and the modified CH3 domain of the second heavy chain comprises amino acid modifications at positions T350, T366, K392 and T394. Preferably, the amino acid modification at position T350 is T350V, T350I, T350L or T350M; the amino acid modification at position L351 is L351Y; the amino acid modification at position F405 is F405A, F405V, F405T or F405S; the amino acid modification at position Y407 is Y407V, Y407A or Y407I; the amino acid modification at position T366 is T366L, T366I, T366V or T366M, the amino acid modification at position K392 is K392F, K392L or K392M, and the amino acid modification at position T394 is T394W. More preferably, the modified heterodimeric CH3 domain of the first heavy chain comprises mutations T350V, L351Y, F405A and Y407V, and the modified heterodimeric CH3 domain of the second heavy chain comprises mutations T350V, T366L, K392L and T394W. The numbering of amino acid residues in the antibody throughout the specification is performed according to the EU index as described in Kabat et al., Sequences of Proteins of Immunological Interest, 5th Ed. Public Health Service, National Institutes of Health, Bethesda, Md. (1991), unless otherwise explicitly stated.

In certain embodiments, the fragment crystallizable region (Fc region) of the second antibody or antigen binding fragment thereof contains substitutions that alter (increase or diminish), preferably eliminate, effector function, such as antibody dependent cellular cytotoxicity (ADCC) and/or complement dependent cytotoxicity (CDC). Preferably, the Fc region of the second antibody or antigen binding fragment thereof comprises one or more amino acid modifications that decrease or abolish the binding of the second antibody or antigen binding fragment thereof to Fc gamma receptors (FcγR) and avoid effector function mediated toxicity. For example, the Fc region of the second antibody or antigen binding fragment thereof can comprise one or more amino acid modifications at positions L234, L235, D270, N297, E318, K320, K322, P331, and P329, such as one, two or three mutations of L234A, L235A and P331S, wherein the numbering of amino acid residues is according to the EU index as set forth in Kabat.

In certain embodiments, the Fc region of the second antibody or antigen binding fragment thereof contains substitutions that alter (increase or diminish), preferably increase, the binding of the second antibody or antigen binding fragment thereof to neonatal Fc receptor (FcRn). Preferably the one or more mutations enhance the binding at an acidic pH, more preferably the Fc has the M252Y/S254T/T256E (YTE) mutations, wherein the numbering of amino acid residues is according to the EU index as set forth in Kabat.

In certain embodiments, a fusion construct of the application comprises:
(1) a first heavy chain having an amino acid sequence that is at least 80%, such as at least 85%, 90%, 95% or 100%, identical to an amino acid sequence selected from the group consisting of SEQ ID NOs: 13, 16, 19, 22, 25, 32, 35, 38, 41, 44, 51, 54, 57, 60, 63, 73, 76, 79, 82, 85, 95, 98, 101, 104, 107, 119, 129, 139, 146, 156, 166, 176, 186, 200, 210, and 219;
(2) two light chains each independently having an amino acid sequence that is at least 80%, such as at least 85%, 90%, 95% or 100%, identical to an amino acid sequence selected from the group consisting of SEQ ID NOs: 14, 17, 20, 23, 26, 33, 36, 42, 45, 52, 55, 58, 61, 64, 74, 77, 80, 83, 86, 96, 99, 102, 105, 108, 120, 130, 140, 147, 157, 167, 177, 187, 201, 211, and 220, respectively; and
(3) a second heavy chain having an amino acid sequence that is at least 80%, such as at least 85%, 90%, 95% or 100%, identical to an amino acid sequence selected from the group consisting of SEQ ID NOs: 15, 18, 21, 24, 27, 34, 37, 43, 46, 53, 56, 59, 62, 65, 75, 78, 81, 84, 87, 97, 100, 103, 106, 109, 121, 131, 141, 148, 158, 168, 178, 188, 202, 212, and 221, respectively.

Another general aspect of the application relates to an isolated nucleic acid encoding the antibody or antigen-binding fragment, a conjugate, or a fusion construct of the application. Also provided is a vector comprising the isolated nucleic acid of the application, a host cell comprising the nucleic acid or the vector of the application.

Another general aspect of the application relates to a method of producing the antibody or antigen-binding fragment, a conjugate, or a fusion construct of the application. The method comprises culturing a cell comprising a nucleic acid of the application under conditions to produce the antibody or antigen-binding fragment, the conjugate or the fusion construct, and recovering the antibody or antigen-binding fragment, the conjugate or the fusion construct from the cell or cell culture.

Further provided is a pharmaceutical composition comprising a conjugate or a fusion construct of the application and a pharmaceutically acceptable carrier.

Another general aspect of the application relates to a method of treating or detecting a neurological disorder in a subject in need thereof, comprising administering to the subject an effective amount of an anti-CD98 antibody or antigen binding fragment thereof, a conjugate or a fusion construct, or a pharmaceutical composition of the application. Preferably, the neurological disorder is selected from the group consisting of neurodegenerative diseases (such as Lewy body disease, postpoliomyelitis syndrome, Shy-Draeger syndrome, olivopontocerebellar atrophy, Parkinson's disease, multiple system atrophy, striatonigral degeneration, spinocerebellar ataxia, spinal muscular atrophy), tauopathies (such as Alzheimer disease and supranuclear palsy), prion diseases (such as bovine spongiform encephalopathy, scrapie, Creutz-feldt-Jakob syndrome, kuru, Gerstmann-Straussler-Scheinker disease, chronic wasting disease, and fatal familial insomnia), bulbar palsy, motor neuron disease, and nervous system heterodegenerative disorders (such as Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (such as Pick's disease, and spinocerebellar ataxia), and cancer of the CNS and/or brain (such as brain metastases resulting from cancer elsewhere in the body).

Preferably, the antibody or antigen binding fragment thereof, the conjugate, the fusion construct or the pharmaceutical composition of the application is administered intravenously.

Also described is a method of delivering a therapeutic or diagnostic agent to the brain of a subject in need thereof, comprising administering to the subject a conjugate comprising the therapeutic or diagnostic agent coupled to an anti-CD98 antibody or antigen-binding fragment thereof of the application. Preferably, the therapeutic or diagnostic agent is a second antibody or an antigen binding fragment thereof that binds to a brain target. More preferably, the administration of the therapeutic or diagnostic agent coupled to an anti-CD98 antibody or antigen-binding fragment thereof of the application to the brain of a subject results in reduced Fc-mediated effector function and/or does not induce rapid reticulocyte depletion, as compared to the administration of the therapeutic or diagnostic agent not coupled to the anti-CD98 antibody or antigen-binding fragment thereof.

Yet another general aspect of the invention relates to a method of inducing antibody-dependent phagocytosis (ADP) without stimulating secretion of a pro-inflammatory cytokine in a subject in need thereof, comprising administering to the subject a complex comprising a therapeutic antibody or antigen binding fragment thereof coupled to, preferably covalently conjugated to, an antigen-binding fragment thereof according to an embodiment of the invention, wherein the therapeutic antibody or antigen binding fragment thereof does not have effector function, for example, the therapeutic antibody or antigen binding fragment thereof comprises one or more amino acid modifications at positions L234, L235, D270, N297, E318, K320, K322, P331, and P329, such as one, two or three mutations of L234A, L235A and P331S, wherein the numbering of amino acid residues is according to the EU index as set forth in Kabat. Preferably, the therapeutic antibody or antigen binding fragment thereof binds specifically to tau aggregates.

Other aspects, features and advantages of the invention will be apparent from the following disclosure, including the detailed description of the invention and its preferred embodiments and the appended claims.

### BRIEF DESCRIPTION OF THE FIGURES

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise embodiments shown in the drawings.

The patent or application file contains at least one drawing executed in color. Copies of this patent or patent application publication with color drawing(s) will be provided by the Office upon request and payment of the necessary fee.

FIG. 1 is an illustration of tripod mAb format used for the brain delivery platform.

FIG. 2 is a bar graph showing the effect of anti-CD98hc Zymeworks tripod antibodies on ³H-Leucine uptake. Data is mean of quadruplicates±SEM. Dotted lines represent ±(3×SD) of Zymeworks Tripod isotype control (B624).

FIG. 3 is an image showing internalization of tripod mAbs in human brain endothelial cells. Tripod mAbs are stained red, nucleus is blue, and actin green. Image shown is for BBBB449.

FIG. 4 is a graph showing pH dependent binding, which was assessed by comparing off-rates at pH 7.4 to off-rate as pH was reduced to 6.5 and 6.0. Tripod mAbs were scored positive if the off-rate was faster as the pH decreased. Data are for BBBB578.

FIG. 5 is an image showing internalization of the tripod mAb BBBB1067, in human brain endothelial cells. Tripod mAbs are stained red and actin green.

FIG. 6 is a graph showing brain concentration of mAbs dosed i.v. All brain shuttle mAbs had increased exposure over the non-brain shuttle mAbs. Individual points represent each animal (n=3).

FIG. 7 is a graph showing mAb mediated uptake into microglial phagosomes. All brain shuttle mAbs promoted similar uptake into phagosomes than the non-brain shuttle mAb, PT1B844.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

Unless otherwise stated, any numerical value, such as a concentration or a concentration range described herein, are to be understood as being modified in all instances by the term “about.” Thus, a numerical value typically includes ±10% of the recited value. For example, a dosage of 10 mg includes 9 mg to 11 mg. As used herein, the use of a numerical range expressly includes all possible subranges, all individual numerical values within that range, including integers within such ranges and fractions of the values unless the context clearly indicates otherwise.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Throughout this specification and the claims which follow, unless the context requires otherwise, the word “comprise,” and variations such as “comprises” and “comprising,” will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising,” “containing,” “including,” and “having,” whenever used herein in the context of an aspect or embodiment of the invention can be replaced with the term “consisting of” or “consisting essentially of” to vary scopes of the disclosure.

The term “antibody” herein is used in the broadest sense and specifically includes full-length monoclonal antibodies, polyclonal antibodies, and, unless otherwise stated or contradicted by context, antigen-binding fragments, antibody variants, and multispecific molecules thereof, so long as they exhibit the desired biological activity. Generally, a full-length antibody is a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarily determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. General principles of antibody molecule structure and various techniques relevant to the production of antibodies are provided in, e.g., Harlow and Lane, ANTIBODIES: A LABORATORY MANUAL, Cold Spring Harbor Laboratory Press, Cold Spring Harbor, N.Y., (1988).

Depending on the amino acid sequence of the constant domain of their heavy chains, full length antibodies can be assigned to different “classes”. There are five major classes of full-length antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known.

An “antibody” can also be a single variable domain on a heavy chain (VHH) antibody, also referred to as a heavy chain only antibody (HcAb), which are devoid of light chains and can be naturally produced by camelids or sharks. The antigen binding portion of the HcAb is comprised of a VHH fragment.

The term “recombinant antibody”, as used herein, refers to an antibody (e.g. a chimeric, humanized, or human antibody or antigen-binding fragment thereof) that is expressed by a recombinant host cell comprising nucleic acid encoding the antibody. Examples of “host cells” for producing recombinant antibodies include: (1) mammalian cells, for example, Chinese Hamster Ovary (CHO), COS, myeloma cells (including YO and NSO cells), baby hamster kidney (BHK), Hela and Vero cells; (2) insect cells, for example, sf9, sf21 and Tn5; (3) plant cells, for example plants belonging to the genus Nicotiana (e.g. Nicotiana tabacum); (4) yeast cells, for example, those belonging to the genus Saccharomyces (e.g. Saccharomyces cerevisiae) or the genus Aspergillus (e.g. Aspergillus niger); (5) bacterial cells, for example Escherichia, coli cells or Bacillus subtilis cells, etc.

An “antigen-binding fragment” of an antibody is a molecule that comprises a portion of a full-length antibody which is capable of detectably binding to the antigen, typically comprising one or more portions of at least the VH region. Antigen-binding fragments include multivalent molecules comprising one, two, three, or more antigen-binding portions of an antibody, and single-chain constructs wherein the VL and VH regions, or selected portions thereof, are joined by synthetic linkers or by recombinant methods to form a functional, antigen-binding molecule. Antigen-binding fragments can also be a single-domain antibody (sdAb), also known as a nanobody, which is an antibody fragment consisting of a single monomeric variable antibody domain (VHH). While some antigen-binding fragments of an antibody can be obtained by actual fragmentation of a larger antibody molecule (e.g., enzymatic cleavage), most are typically produced by recombinant techniques. The antibodies of the invention can be prepared as full-length antibodies or antigen-binding fragments thereof. Examples of antigen-binding fragments include Fab, Fab′, F(ab)₂, F(ab′)₂, F(ab)₃, Fv (typically the VL and VH domains of a single arm of an antibody), single-chain Fv (scFv, see e.g., Bird et al., Science 1988; 242:423-426; and Huston et al. PNAS 1988; 85:5879-5883), dsFv, Fd (typically the VH and CH1 domain), and dAb (typically a VH domain) fragments; VH, VL, VHH, and V-NAR domains; monovalent molecules comprising a single VH and a single VL chain; minibodies, diabodies, triabodies, tetrabodies, and kappa bodies (see, e.g., Ill et al., Protein Eng 1997; 10:949-57); camel IgG; IgNAR; as well as one or more isolated CDRs or a functional paratope, where the isolated CDRs or antigen-binding residues or polypeptides can be associated or linked together so as to form a functional antibody fragment. Various types of antibody fragments have been described or reviewed in, e.g., Holliger and Hudson, Nat Biotechnol 2005; 23:1126-1136; WO2005040219, and published U.S. Patent Applications 20050238646 and 20020161201. Antibody fragments can be obtained using conventional recombinant or protein engineering techniques, and the fragments can be screened for antigen-binding or other function in the same manner as are intact antibodies.

Various techniques have been developed for the production of antibody fragments. Traditionally, these fragments were derived via proteolytic digestion of full-length antibodies (see, e.g., Morimoto et al., Journal of Biochemical and Biophysical Methods, 24:107-117 (1992); and Brennan et al., Science, 229:81 (1985)). However, these fragments can now be produced directly by recombinant host cells. Alternatively, Fab′-SH fragments can be directly recovered from E. coli and chemically coupled to form F(ab′)₂ fragments (Carter et al., Bio/Technology, 10:163-167 (1992)). According to another approach, F(ab′)₂ fragments can be isolated directly from recombinant host cell culture. In other embodiments, the antibody of choice is a single-chain Fv fragment (scFv). See WO 1993/16185; U.S. Pat. Nos. 5,571,894; and 5,587,458. The antibody fragment may also be a “linear antibody”, e.g., as described in U.S. Pat. No. 5,641,870, for example. Such linear antibody fragments can be monospecific or bispecific.

The term “antibody derivative” as used herein refers to a molecule comprising a full-length antibody or an antigen-binding fragment thereof, wherein one or more amino acids are chemically modified or substituted. Chemical modifications that can be used in antibody derivative includes, e.g., alkylation, PEGylation, acylation, ester formation or amide formation or the like, e.g., for linking the antibody to a second molecule. Exemplary modifications include PEGylation (e.g., cysteine-PEGylation), biotinylation, radiolabeling, and conjugation with a second agent (such as a cytotoxic agent).

Antibodies herein include “amino acid sequence variants” with altered antigen-binding or biological activity. Examples of such amino acid alterations include antibodies with enhanced affinity for antigen (e.g. “affinity matured” antibodies), and antibodies with altered Fc region, if present, e.g. with altered (increased or diminished) antibody dependent cellular cytotoxicity (ADCC) and/or complement dependent cytotoxicity (CDC) (see, for example, WO 00/42072, Presta, L. and WO 99/51642, Iduosogie et al); and/or increased or diminished serum half-life (see, for example, WO00/42072, Presta, L.).

A “multispecific molecule” comprises an antibody, or an antigen-binding fragment thereof, which is associated with or linked to at least one other functional molecule (e.g. another peptide or protein such as another antibody or ligand for a receptor) thereby forming a molecule that binds to at least two different binding sites or target molecules. Exemplary multispecific molecules include bi-specific antibodies and antibodies linked to soluble receptor fragments or ligands.

The term “human antibody”, as used herein, is intended to include antibodies having variable regions in which both the framework and CDR regions are derived from (i.e., are identical or essentially identical to) human germline immunoglobulin sequences. Furthermore, if the antibody contains a constant region, the constant region also is “derived from” human germline immunoglobulin sequences. The human antibodies of the invention can include amino acid residues not encoded by human germline immunoglobulin sequences (e.g., mutations introduced by random or site-specific mutagenesis in vitro or by somatic mutation in viva). However, the term “human antibody”, as used herein, is not intended to include antibodies in which CDR sequences derived from the germline of another mammalian species, such as a mouse, have been grafted onto human framework sequences.

A “humanized” antibody is a human/non-human chimeric antibody that contains a minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or non-human primate having the desired specificity, affinity, and capacity. In some instances, FR residues of the human immunoglobulin are replaced by corresponding non-human residues. Furthermore, humanized antibodies can comprise residues that are not found in the recipient antibody or in the donor antibody. These modifications are made to further refine antibody performance. In general, a humanized antibody will comprise substantially all of at least one, and typically two, variable domains, in which all or substantially all of the hypervariable loops correspond to those of a non-human immunoglobulin and all or substantially all of the FR residues are those of a human immunoglobulin sequence. The humanized antibody can optionally also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992), WO 92/02190, US Patent Application 20060073137, and U.S. Pat. Nos. 6,750,325, 6,632,927, 6,639,055, 6,548,640, 6,407,213, 6,180,370, 6,054,297, 5,929,212, 5,895,205, 5,886,152, 5,877,293, 5,869,619, 5,821,337, 5,821,123, 5,770,196, 5,777,085, 5,766,886, 5,714,350, 5,693,762, 5,693,761, 5,530,101, 5,585,089, and 5,225,539.

The term “hypervariable region” when used herein refers to the amino acid residues of an antibody that are responsible for antigen binding. The hypervariable region generally comprises amino acid residues from a “complementarity-determining region” or “CDR” (residues 24-34 (L1), 50-56 (L2) and 89-97 (L3) in the light-chain variable domain and 31-35 (H1), 50-65 (H2) and 95-102 (H3) in the heavy-chain variable domain; (Kabat et al. (1991) Sequences of Proteins of Immunological Interest, Fifth Edition, U.S. Department of Health and Human Services, NIH Publication No. 91-3242) and/or those residues from a “hypervariable loop” (residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol. 1987; 196:901-917). Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide can contain fewer or additional amino acids corresponding to a shortening of, or insertion into, a FR or CDR of the variable domain. For example, a heavy chain variable domain can include a single amino acid insert (residue 52a according to Kabat) after residue 52 of CDR H2 and inserted residues (e.g. residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence.

“Framework region” or “FR” residues are those VH or VL residues other than the CDRs as herein defined.

An “epitope” or “binding site” is an area or region on an antigen to which an antigen-binding peptide (such as an antibody) specifically binds. A protein epitope can comprise amino acid residues directly involved in the binding (also called the immunodominant component of the epitope) and other amino acid residues, which are not directly involved in the binding, such as amino acid residues which are effectively blocked by the specifically antigen binding peptide (in other words, the amino acid residue is within the “solvent-excluded surface” and/or “footprint” of the specifically antigen binding peptide).

A “paratope” is an area or region of an antigen-binding portion of an antibody that specifically binds an antigen. Unless otherwise stated or clearly contradicted by context, a paratope can comprise amino acid residues directly involved in epitope binding, several of which are typically in CDRs, and other amino acid residues, which are not directly involved in the binding, such as amino acid residues which are effectively blocked by the specifically bound antigen (in other words, the amino acid residue is within the “solvent-excluded surface” and/or “footprint” of the specifically bound antigen).

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For a review of methods for assessment of antibody purity, see, e.g., Flatman et al, J. Chromatogr. B 848:79-87 (2007).

The term “administering” with respect to the methods of the invention, means a method for therapeutically or prophylactically preventing, treating or ameliorating a syndrome, disorder or disease as described herein by using a conjugate of the invention or a form, composition or medicament thereof. Such methods include administering an effective amount of said antibody, antigen-binding fragment thereof, or conjugate, or a form, composition or medicament thereof at different times during the course of a therapy or concurrently in a combination form. The methods of the invention are to be understood as embracing all known therapeutic treatment regimens.

The ability of a target antibody to “block” the binding of a target molecule to a natural target ligand, means that the antibody, in an assay using soluble or cell-surface associated target and ligand molecules, can detectably reduce the binding of a target molecule to the ligand in a dose-dependent fashion, where the target molecule detectably binds to the ligand in the absence of the antibody.

The “blood-brain barrier” or “BBB” refers a physiological barrier between the peripheral circulation and the brain and spinal cord which is formed by tight junctions within the brain capillary endothelial plasma membranes, creating a tight barrier that restricts the transport of molecules into the brain. The BBB can restrict the transport of even very small molecules such as urea (60 Daltons) into the brain. Examples of the BBB include the BBB within the brain, the blood-spinal cord barrier within the spinal cord, and the blood-retinal barrier within the retina, all of which are contiguous capillary barriers within the CNS. The BBB also encompasses the blood-CSF barrier (choroid plexus) where the barrier is comprised of ependymal cells rather than capillary endothelial cells.

A “blood-brain barrier receptor” (abbreviated “R/BBB” herein) is an extracellular membrane-linked receptor protein expressed on brain endothelial cells which is capable of transporting molecules across the BBB or be used to transport exogenous administrated molecules. Examples of RBBB include, but are not limited to, Large neutral Amino acid Transporter (LAT) complex, including CD98 component, transferrin receptor (TfR), insulin receptor, insulin-like growth factor receptor (IGF-R), low density lipoprotein receptors including without limitation low density lipoprotein receptor-related protein 1 (LRP1) and low density lipoprotein receptor-related protein 8 (LRP8), and heparin-binding epidermal growth factor-like growth factor (HB-EGF). An exemplary R/BBB herein is CD98hc.

The “central nervous system” or “CNS” refers to the complex of nerve tissues that control bodily function, and includes the brain and spinal cord.

A “conjugate” as used herein refer to a protein covalently linked to one or more heterologous molecule(s), including but not limited to a therapeutic peptide or protein, an antibody, a label, or a neurological disorder drug.

As used herein the term “coupled” refers to the joining or connection of two or more objects together. When referring to chemical or biological compounds, coupled can refer to a covalent connection between the two or more chemical or biological compounds. By way of a non-limiting example, an antibody of the invention can be coupled with a peptide of interest to form an antibody coupled peptide. An antibody coupled peptide can be formed through specific chemical reactions designed to conjugate the antibody to the peptide. In certain embodiments, an antibody of the invention can be covalently coupled with a peptide of the invention through a linker. The linker can, for example, be first covalently connected to the antibody or the peptide, then covalently connected to the peptide or the antibody.

An “effective amount” or “therapeutically effective amount” of an agent, e.g., a pharmaceutical formulation, refers to an amount effective, at dosages and for periods of time necessary, to achieve the desired therapeutic or prophylactic result.

A “linker” as used herein refers to a chemical linker or a single chain peptide linker that covalently connects two different entities. A linker can be used to connect any two of an antibody or a fragment thereof, a blood brain barrier shuttle, a fusion protein and a conjugate of the present invention. The linker can connect, for example, the VH and VL in scFv, or the monoclonal antibody or antigen-binding fragment thereof with a therapeutic molecule, such as a second antibody. In some embodiment, if the monovalent binding entity comprises a scFv directed to CD98, preferably human CD98hc, and the therapeutic molecule comprises an antibody directed to a CNS target, such as Tau, then the linker can connect the scFv to the antibody directed to Tau. Single chain peptide linkers, comprised of from 1 to 25 amino acids, such as 1, 2, 3, 4, 5, 6, 7, 8, 9, 10, 11, 12, 13, 14, 15, 16, 17, 18, 19, 20, 21, 22, 23, 24, or 25 amino acids, joined by peptide bonds, can be used. In certain embodiments, the amino acids are selected from the twenty naturally occurring amino acids. In certain other embodiments, one or more of the amino acids are selected from glycine, alanine, proline, asparagine, glutamine and lysine. Chemical linkers, such as a hydrocarbon linker, a polyethylene glycol (PEG) linker, a polypropylene glycol (PPG) linker, a polysaccharide linker, a polyester linker, a hybrid linker consisting of PEG and an embedded heterocycle, and a hydrocarbon chain can also be used.

A “neurological disorder” as used herein refers to a disease or disorder which affects the CNS and/or which has an etiology in the CNS. Exemplary CNS diseases or disorders include, but are not limited to, neuropathy, amyloidosis, cancer, an ocular disease or disorder, viral or microbial infection, inflammation, ischemia, neurodegenerative disease, seizure, behavioral disorders, and a lysosomal storage disease. For the purposes of this application, the CNS will be understood to include the eye, which is normally sequestered from the rest of the body by the blood-retina barrier. Specific examples of neurological disorders include, but are not limited to, neurodegenerative diseases (including, but not limited to, Lewy body disease, postpoliomyelitis syndrome, Shy-Draeger syndrome, olivopontocerebellar atrophy, Parkinson's disease, multiple system atrophy, striatonigral degeneration, spinocerebellar ataxia, spinal muscular atrophy), tauopathies (including, but not limited to, Alzheimer disease and supranuclear palsy), prion diseases (including, but not limited to, bovine spongiform encephalopathy, scrapie, Creutz-feldt-Jakob syndrome, kuru, Gerstmann-Straussler-Scheinker disease, chronic wasting disease, and fatal familial insomnia), bulbar palsy, motor neuron disease, and nervous system heterodegenerative disorders (including, but not limited to, Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (including, but not limited to, Pick's disease, and spinocerebellar ataxia), cancer (e.g. of the CNS and/or brain, including brain metastases resulting from cancer elsewhere in the body).

A “neurological disorder drug” is a drug or therapeutic agent useful in treating or ameliorating the effects of one or more neurological disorder(s). Neurological disorder drugs of the invention include, but are not limited to, small molecule compounds, antibodies, peptides, proteins, natural ligands of one or more CNS target(s), modified versions of natural ligands of one or more CNS target(s), aptamers, inhibitory nucleic acids (i.e., small inhibitory RNAs (siRNA) and short hairpin RNAs (shRNA)), ribozymes, or active fragments of any of the foregoing. Exemplary neurological disorder drugs of the invention are described herein and include, but are not limited to: antibodies, aptamers, proteins, peptides, inhibitory nucleic acids and small molecules and active fragments of any of the foregoing that either are themselves or specifically recognize and/or act upon (i.e., inhibit, activate, or detect) a CNS antigen or target molecule such as, but not limited to, amyloid precursor protein or portions thereof, amyloid beta, beta-secretase, gamma-secretase, tau, alpha-synuclein, parkin, huntingtin, DR6, presenilin, ApoE, glioma or other CNS cancer markers, and neurotrophins. Non-limiting examples of neurological disorder drugs and the corresponding disorders they may be used to treat: Brain-derived neurotrophic factor (BDNF), Chronic brain injury (Neurogenesis), Fibroblast growth factor 2 (FGF-2), Anti-Epidermal Growth Factor Receptor Brain cancer, (EGFR)-antibody, Glial cell-line derived neural factor Parkinson's disease, (GDNF), Brain-derived neurotrophic factor (BDNF) Amyotrophic lateral sclerosis, depression, Lysosomal enzyme Lysosomal storage disorders of the brain, Ciliary neurotrophic factor (CNTF) Amyotrophic lateral sclerosis, Neuregulin-1 Schizophrenia, Anti-HER2 antibody (e.g. trastuzumab) Brain metastasis from HER2-positive cancer.

The term “pharmaceutical formulation” refers to a preparation which is in such form as to permit the biological activity of an active ingredient contained therein to be effective, and which contains no additional components which are unacceptably toxic to a subject to which the formulation would be administered.

As used herein, “pharmaceutically acceptable carrier or diluent” means any substance suitable for use in administering to an individual. For example, a pharmaceutically acceptable carrier can be a sterile aqueous solution, such as phosphate buffer saline (PBS) or water-for-injection.

As used herein, “pharmaceutically acceptable salts” means physiologically and pharmaceutically acceptable salts of compounds, such as oligomeric compounds or oligonucleotides, i.e., salts that retain the desired biological activity of the parent compound and do not impart undesired toxicological effects thereto.

Pharmaceutically acceptable acidic/anionic salts for use in the invention include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide. Organic or inorganic acids also include, and are not limited to, hydriodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic or trifluoroacetic acid. Pharmaceutically acceptable basic/cationic salts include, and are not limited to aluminum, 2-amino-2-hydroxymethyl-propane-1,3-diol (also known as tris(hydroxymethyl)aminomethane, tromethane or “TRIS”), ammonia, benzathine, t-butylamine, calcium, chloroprocaine, choline, cyclohexylamine, diethanolamine, ethylenediamine, lithium, L-lysine, magnesium, meglumine, N-methyl-D-glucamine, piperidine, potassium, procaine, quinine, sodium, triethanolamine, or zinc.

“Polypeptide” or “protein” means a molecule that comprises at least two amino acid residues linked by a peptide bond to form a polypeptide. Small polypeptides of less than 50 amino acids may be referred to as “peptides”.

The phrases “sequence identity” or “percent (%) sequence identity” or “% identity” or “% identical to” when used with reference to an amino acid sequence describe the number of matches (“hits”) of identical amino acids of two or more aligned amino acid sequences as compared to the number of amino acid residues making up the overall length of the amino acid sequences. In other terms, using an alignment, for two or more sequences the percentage of amino acid residues that are the same (e.g. 90%, 91%, 92%, 93%, 94%, 95%, 97%, 98%, 99%, or 100% identity over the full-length of the amino acid sequences) may be determined, when the sequences are compared and aligned for maximum correspondence as measured using a sequence comparison algorithm as known in the art, or when manually aligned and visually inspected. The sequences which are compared to determine sequence identity may thus differ by substitution(s), addition(s) or deletion(s) of amino acids. Suitable programs for aligning protein sequences are known to the skilled person. The percentage sequence identity of protein sequences can, for example, be determined with programs such as CLUSTALW, Clustal Omega, FASTA or BLAST, e.g. using the NCBI BLAST algorithm (Altschul S F, et al (1997), Nucleic Acids Res. 25:3389-3402).

“Specific binding” or “specifically binds” or “binds” refer to antibody binding to an antigen or an epitope within the antigen with greater affinity than for other antigens. Typically, the antibody binds to the antigen or the epitope within the antigen with a dissociation constant (K_{D}) of about 1×10⁻⁸ M or less, for example about 1×10⁻⁹ M or less, about 1×10⁻¹⁰ M or less, about 1×10⁻¹¹ M or less, or about 1×10⁻¹² M or less, typically with a K_{D} that is at least one hundred fold less than its K_{D} for binding to a non-specific antigen (e.g., BSA, casein). K_{D} is the equilibrium dissociation constant, a ratio of k_{off}/kₒₙ, between the antibody and its antigen. K_{D} and affinity are inversely related. The “on-rate” (kₒₙ) is a constant used to characterize how quickly the antibody binds to its target. The “off-rate” (k_{off}) is a constant used to characterize how quickly an antibody dissociates from its target. The dissociation constant K_{D} can be measured using standard procedures. For example, the K_{D} of an antibody can be determined by using surface plasmon resonance, such as by using a biosensor system, e.g., a Biacore® system, or by using bio-layer interferometry technology, such as an Octet RED96 system. The smaller the value of the K_{D} of an antibody, the higher affinity that the antibody binds to a target antigen. Antibodies that specifically bind to the antigen or the epitope within the antigen can, however, have cross-reactivity to other related antigens, for example to the same antigen from other species (homologs), such as human or monkey, for example Macaca fascicularis (cynomolgus, cyno), Pan troglodytes (chimpanzee, chimp) or Callithrix jacchus (common marmoset, marmoset). While a monospecific antibody specifically binds one antigen or one epitope, a bispecific antibody specifically binds two distinct antigens or two distinct epitopes.

The term “subject” as used herein refers to a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. When the subject is human, they can also be referred to as a “patient”.

The term “substantially identical” in the context of two amino acid sequences means that the sequences, when optimally aligned, such as by the programs GAP or BESTFIT using default gap weights, share at least about 50 percent sequence identity. Typically sequences that are substantially identical will exhibit at least about 60, at least about 70, at least about 80, at least about 90, at least about 95, at least about 98, or at least about 99 percent sequence identity.

The term “CD98” or “CD98hc” as used herein, refers to an integral membrane protein consisting of a cluster of differentiation 98 heavy chain (CD98hc) that links to any of multiple light chains by a disulfide bond. When associated with LAT1 or LAT2, the heterodimer transporter complexes behave as obligatory amino acid exchangers. CD98hc has a molecular weight of about 80 kDa. Preferably, the CD98hc is a human CD98hc (huCD98hc). huCD98hc is encoded by the SLC3A2 gene.

A “target antigen” or “brain target,” as used herein, refers to an antigen and/or molecule expressed in the CNS, including the brain, which can be targeted with an antibody or small molecule. Examples of such antigens and/or molecules include, without limitation: beta-secretase 1 (BACE1), amyloid beta (Abeta), epidermal growth factor receptor (EGFR), human epidermal growth factor receptor 2 (HER2), Tau, apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), and caspase 6. In some embodiments, the target antigen is BACE1. In some embodiments, the target antigen is Tau.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

Antibodies or immunoglobulins can be assigned to five major classes, namely IgA, IgD, IgE, IgG and IgM, depending on the heavy chain constant domain amino acid sequence. IgG is the most stable of the five types of immunoglobulins, having a serum half-life in humans of about 23 days. IgA and IgG are further sub-classified as the isotypes IgA₁, IgA₂, IgG₁, IgG₂, IgG₃ and IgG₄. Each of the four IgG subclasses has different biological functions known as effector functions. These effector functions are generally mediated through interaction with the Fc receptor (FcγR) and/or by binding C1q and fixing complement. Binding to FcγR can lead to antibody dependent cell mediated cytolysis or antibody-dependent cellular cytotoxicity (ADCC), whereas binding to complement factors can lead to complement mediated cell lysis or complement-dependent cytotoxicity (CDC). An anti-CD98 antibody of the invention, or a therapeutic or diagnostic antibody to be conjugated or fused to the anti-CD98 antibody can have no or minimal effector function, but retains its ability to bind FcRn, the binding of which can be a primary means by which antibodies have an extended in vivo half-life.

Binding of FcγR or complement (e.g., C1q) to an antibody is caused by defined protein-protein interactions at the so-called Fc part binding site. Such Fc part binding sites are known in the art. Such Fc part binding sites include, e.g., characterized by the amino acids L234, L235, D270, N297, E318, K320, K322, P331, and P329 (numbering according to EU index of Kabat). In some embodiments, an anti-CD98 antibody of the invention, or a therapeutic or diagnostic antibody to be conjugated or fused to the anti-CD98 antibody contains one or more substitutions in one or more Fc part binding sites to eliminate the effector function. For example, an anti-CD98 antibody of the invention, or a therapeutic or diagnostic antibody to be conjugated or fused to the anti-CD98 antibody can contain a Fc region containing one or more of the following substitutions: substitution of proline for glutamate at residue 233, alanine or valine for phenylalanine at residue 234 and alanine or glutamate for leucine at residue 235 (EU numbering, Kabat, E. A. et al. (1991) Sequences of Proteins of Immunological Interest, 5th Ed. U.S. Dept. of Health and Human Services, Bethesda, Md., NIH Publication no. 91-3242). Preferably, the antibody of interest contains one, two or three mutations of L234A, L235A and P331S (EU numbering, Kabat).

Antibodies of subclass IgG1, IgG2, and IgG3 usually show complement activation including C1q and C3 binding, whereas IgG4 does not activate the complement system and does not bind C1q and/or C3. Human IgG4 Fc region has reduced ability to bind FcγR and complement factors compared to other IgG sub-types. Preferably, an anti-CD98 antibody of the invention, or a therapeutic or diagnostic antibody to be conjugated or fused to the anti-CD98 antibody comprises a Fc region derived from human IgG4 Fc region. More preferably, the Fc region contains human IgG4 Fc region having substitutions that eliminate effector function. For example, removing the N-linked glycosylation site in the IgG4 Fc region by substituting Ala for Asn at residue 297 (EU numbering) is another way to ensure that residual effector activity is eliminated.

### Anti-CD98 Antibodies and Antigen Binding Fragments Thereof

In one general aspect, the application relates to an antibody or an antigen binding fragment thereof that binds to a primate CD98, such as a human CD98 or a monkey CD98, and the antibody or an antigen binding fragment thereof is optimized for delivering an agent to the brain of a subject in need thereof. The inventors of the present invention surprisingly discovered a more nuanced relationship between affinity and transcytosis efficiency than what has been previously described, with influence from both on- and off-rates impacting brain concentration. In particular, a neutral off-rate that is neither too fast nor too slow is required for optimal brain PK and PD of an agent (such as an mAb) to be efficiently delivered by the anti-CD98 antibody or antigen binding fragment thereof.

Preferably, an anti-CD98 antibody or antigen binding fragment thereof of the application is pH-sensitive, e.g., it has different binding affinities to CD98 at different pHs. For example, an anti-CD98 antibody of the application can bind to CD98 at a neutral pH, such as physiological pH (e.g., pH 7.4), with high affinity, but upon internalization into an endosomal compartment, dissociates from CD98 at an acidic pH, such as the relatively lower pH (pH 5-6.0). Affinity is a measure of the strength of binding between two moieties, e.g., an antibody and an antigen. Affinity can be expressed in several ways. One way is in terms of the dissociation constant (K_{D}) of the interaction. K_{D} can be measured by routine methods, include equilibrium dialysis or by directly measuring the rates of antigen-antibody dissociation and association, the k_{off}(kd or k_{dis}) and kₒₙ (or ka) rates, respectively (see e.g., Nature, 1993 361:186-87). The ratio of k_{off}/kₒₙ cancels all parameters not related to affinity, and is equal to the dissociation constant K_{D} (see, generally, Davies et al., Annual Rev Biochem, 1990 59:439-473). Thus, a smaller K_{D} means a higher affinity. Another expression of affinity is Kₐ, which is the inverse of K_{D}, or kₒₙ/k_{off}. Thus, a higher Kₐ means a higher affinity. For example, an antibody or antigen binding fragment thereof for use in a composition and/or method of the application can be an antibody or fragment thereof that binds to a CD98 with a K_{D} of 1 nanomolar (nM, 10⁻⁹ M) or more at a neutral pH (e.g., pH 6.8-7.8), such as a physiological pH (e.g., pH 7.4), and dissociates from CD98 with a k_{dis} of 10⁻⁴ sec⁻¹ or more at an acidic pH (e.g., pH 4.5-6.5), such as pH 5.0.

Accordingly, a general aspect of the application relates to an anti-CD98 antibody or antigen-binding fragment thereof for delivering an agent to the brain of a subject in need thereof, wherein the anti-CD98 antibody or antigen-binding fragment thereof binds to a CD98, preferably a CD98hc, more preferably a human CD98hc, with a dissociation constant K_{D} of at least 1 nM, preferably 1 nM to 500 nM, at neutral pH and an off-rate constant k_{d} of at least 10⁻⁴ sec⁻¹, preferably 10⁻⁴ to 10⁻¹ sec⁻¹, at an acidic pH, preferably pH 5.

In one embodiment, the anti-CD98 antibody or antigen-binding fragment thereof of the application has an off-rate constant k_{d} of 2×10⁻² to 2×10⁻⁴ sec⁻¹, such as 2×10⁻², 1×10⁻², 9×10⁻³, 8×10⁻³, 7×10⁻³, 6×10⁻³, 5×10⁻³, 4×10⁻³, 3×10⁻³, 2×10⁻³, 1×10⁻³, 9×10⁻⁴, 8×10⁻⁴, 7×10⁻⁴, 6×10⁻⁴, 5×10⁻⁴, 4×10⁻⁴, 3×10⁻⁴, 2×10⁻⁴ sec⁻¹, or any value in between, at the neutral pH.

In certain embodiments, the antibody or antigen binding fragment thereof that binds to human CD98 is a single variable domain on a heavy chain (VHH) antibody comprising heavy chain complementarity determining regions (HCDRs) HCDR1, HCDR2 and HCDR3 having the amino acid sequences of:
(i) SEQ ID NOs: 29, 30 and 31, respectively;
(ii) SEQ ID NOs: 48, 49 and 50, respectively; or
(iii) SEQ ID NOs: 143, 144 and 145, respectively.

Preferably, it is a VHH fragment comprising an amino acid sequence having at least 80%, such as at least 85%, 90%, 91%, 92%, 93%, 94%, 95%, 96%, 97%, 98%, 99% or 100%, sequence identity to SEQ ID NO: 28, SEQ ID NO: 47, or SEQ ID NO: 142.

In other embodiments, the antibody or antigen binding fragment thereof that binds to human CD98 comprises a heavy chain variable region comprising heavy chain complementarity determining regions (HCDRs) HCDR1, HCDR2 and HCDR3, and a light chain variable region comprising light chain complementarity determining regions (LCDRs) LCDR1, LCDR2 and LCDR3, wherein the HCDR1, HCDR2, HCDR3, LCDR1, LCDR2 and LCDR3 have the amino acid sequences of:
(i) SEQ ID NOs: 7, 8, 9, 10, 11 and 12, respectively;
(ii) SEQ ID NOs: 67, 68, 69, 70, 71 and 72, respectively;
(iii) SEQ ID NOs: 89, 90, 91, 92, 93 and 94, respectively;
(iv) SEQ ID NOs: 113, 114, 115, 116, 117 and 118, respectively;
(v) SEQ ID NOs: 123, 124, 125, 126, 127 and 128, respectively;
(vi) SEQ ID NOs: 133, 134, 135, 136, 137 and 138, respectively;
(vii) SEQ ID NOs: 150, 151, 152, 153, 154 and 155, respectively;
(viii) SEQ ID NOs: 160, 161, 162, 163, 164 and 165, respectively;
(ix) SEQ ID NOs: 170, 171, 172, 173, 174 and 175, respectively;
(x) SEQ ID NOs: 180, 181, 182, 183, 184 and 185, respectively;
(xi) SEQ ID NOs: 194, 195, 196, 197, 198 and 199, respectively; or
(xii) SEQ ID NOs: 204, 205, 206, 207, 208 and 209, respectively.

In other embodiments, an antibody or antigen binding fragment thereof of the application competes with an antibody or antigen binding fragment exemplified herein. The binding site of an antibody or antigen can be determined by known methods such as ELISA, Western blot, etc. In certain embodiments, such a competing antibody binds to the same epitope (e.g., a linear or a conformational epitope) that is bound by an exemplified antibody or antigen binding fragment thereof. Detailed exemplary methods for mapping an epitope to which an antibody binds are provided in Morris, G. E., (ed.), “Epitope Mapping Protocols,” In: Methods in Molecular Biology, Vol. 66, Humana Press, Totowa, N.J. (1996). An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

Preferably, the antibody or antigen-binding fragment thereof is single-chain variable fragment (scFv) comprising the heavy chain variable region (Hv) covalently linked to the light chain variable region (Lv) via a flexible linker. The scFv can retain the specificity of the original immunoglobulin, despite removal of the constant regions and the introduction of the linker. In a scFv, the order of the domains can be either Hv-linker-Lv, or Lv-linker-Hv. The linker can be designed de novo or derived from known protein structure to provide a compatible length and conformational in bridging the variable domains of a scFv without serious steric interference. The linker can have 10 to about 25 amino acids in length. Preferably, the linker is a peptide linker spanning about 3.5 nm (35 Å) between the carboxy terminus of the variable domain and the amino terminus of the other domain without affecting the ability of the domains to fold and form an intact antigen-binding site (Huston et al., Methods in Enzymology, vol. 203, pp. 46-88, 1991, which is incorporated herein by reference in its entirety). The linker preferably comprises a hydrophilic sequence in order to avoid intercalation of the peptide within or between the variable domains throughout the protein folding (Argos, Journal of Molecular Biology, vol. 211, no. 4, pp. 943-958, 1990). For example, the linker can comprise Gly and Ser residues and/or together with the charged residues such as Glu, Thr and Lys interspersed to enhance the solubility. In one embodiment, the linker has the amino acid sequence of SEQ ID NO: 189 (GTEGKSSGSGSESKST). Any other suitable linker can also be used in view of the present disclosure.

In some embodiments, the scFv comprises an amino acid sequence having at least 80%, such as at least 85%, 90%, 91%, 92%, 93%, 94%, 95%, 96%, 97%, 98%, 99% or 100%, sequence identity to the amino acid sequences of SEQ ID NO: 6, SEQ ID NO: 66, SEQ ID NO: 88, SEQ ID NO: 112, SEQ ID NO: 122, SEQ ID NO: 132, SEQ ID NO: 149, SEQ ID NO: 159, SEQ ID NO: 169, SEQ ID NO: 179, SEQ ID NO: 193, or SEQ ID NO: 203.

In a preferred embodiment, an antibody or antigen binding fragment thereof that binds to CD98, preferably CD98hc, more preferably human CD98hc, does not contain a free cysteine.

An anti-CD98 antibody or antigen-binding fragment thereof (such as a VHH or scFv fragment) can be produced using suitable methods in the art in view of the present disclosure.

For example, a VHH or scFv fragment can be recombinantly produced by growing a recombinant host cell (such as a bacterial, yeast or mammalian cell) under suitable conditions for the production of the antibody fragment and recovering the fragment from the cell culture.

Brain Shuttle Construct

An optimized RMT brain delivery platform is developed using CD98 by enhancing the intrinsic transcytosis efficiency, extending peripheral pharmacokinetics, and engineering for an acceptable safety profile while maintaining efficacy of the therapeutic mAb. The interplay between transcytosis receptor affinity and brain concentration in cynomolgus monkey is studied. A thorough study of binding kinetics demonstrate that for optimal brain PK and PD of mAbs, a neutral off-rate that is neither too fast nor too slow is required.

It is also discovered that engineered antibody constant region with increased binding to the neonatal Fc receptor (FcRn) resulted in decreased peripheral clearance and enhancement in brain concentration.

Additional Fc mutations are introduced to abolish binding to Fc gamma receptors (FcγR) and avoid effector function mediated toxicity. When coupled with a high affinity anti-Tau binding mAb, these mutations prevent effector function mediated toxicity in the periphery while maintaining antibody dependent phagocytosis (ADP) through a novel, non-FcγR mechanism for microglial uptake and target degradation. This mechanism is dependent upon internalization through CD98 and is more efficient in promoting target degradation than traditional FcγR mediated ADP without the stimulating the secretion of pro-inflammatory cytokines. To the knowledge of the inventors, this is the first report of non-FcγR mediated ADP, representing a novel, efficient, non-inflammatory mechanism for phagocytosis that can be exploited for a variety of therapeutic applications.

Accordingly, in one general aspect, the application relates to an antibody-targeted brain delivery system comprising an anti-CD98 antibody or antigen binding fragment thereof of the application. The anti-CD98 antibody or antigen binding fragment thereof can be used to deliver a therapeutic or diagnostic agent into a cell (e.g., a cancer cell) or a BBB system. Agents that can be delivered include any neurological disorder drug or agent that can be used to detect or analyze a neurological disorder drug. For example, such agent can be neurotrophic factors, including, but not limited to, nerve growth factor (NGF), brain derived neurotrophic factor (BDNF), ciliary neurotrophic factor (CNTF), glial cell-line neurotrophic factor (GDNF) and insulin-like growth factor (IGF); neuropeptides, including, but not limited to, Substance P, neuropeptide Y, vasoactive intestinal peptide (VIP), gamma-amino-butyric acid (GABA), dopamine, cholecystokinin (CCK), endorphins, enkephalins and thyrotropin releasing hormone (TRH); cytokines; anxiolytic agents; anticonvulsants; polynucleotides and transgenes, including, for example, small interfering RNAs and/or antisense oligos; or antibodies or antigen binding fragments thereof that bind to a brain target. An anti-CD98 antibody or antigen binding fragment thereof of the application can be an effective means to enhance the delivery of an agent of interest from the blood into the brain and function there.

In particular, an agent of interest can be delivered in a combined form or linked to an anti-CD98 antibody or antigen binding fragment thereof of the application, parenterally, e.g., intravenously. For example, the agent can be non-covalently attached to the anti-CD98 antibody or antigen binding fragment thereof. The agent can also be covalently attached to the anti-CD98 antibody or antigen binding fragment thereof to form a conjugate. In certain embodiments, the conjugation is by construction of a protein fusion (i.e., by genetic fusion of the two genes encoding an anti-CD98 antibody or antigen binding fragment thereof and a neurological disorder drug and expression as a single protein). Known methods can be used to link an agent to an antibody or antigen binding fragment thereof in view of the present disclosure. See, for example, Wu et al., Nat Biotechnol., 23(9):1137-46, 2005; Trail et al., Cancer Immunol Immunother., 52(5):328-37, 2003; Saito et al., Adv Drug Deliv Rev., 55(2):199-215, 2003; Jones et al., Pharmaceutical Research, 24(9):1759-1771, 2007.

In some embodiments, a therapeutic or diagnostic agent to be delivered to the brain and an anti-CD98 antibody or antigen binding fragment thereof can be covalently linked together (or conjugated) via a non-peptide linker or a peptide linker. Examples of non-peptide linkers include, but are not limited to, polyethylene glycol, polypropylene glycol, copolymer of ethylene glycol and propylene glycol, polyoxyethylated polyol, polyvinyl alcohol, polysaccharides, dextran, polyvinyl ether, biodegradable polymer, polymerized lipid, chitins, and hyaluronic acid, or derivatives thereof, or combinations thereof. A peptide linker can be a peptide chain consisting of 1 to 50 amino acids linked by peptide bonds or a derivative thereof, whose N-terminus and C-terminus can be covalently linked to an anti-CD98 antibody or an antigen binding fragment thereof.

In certain embodiments, a conjugate of the application is a multi-specific antibody comprising a first antigen binding region which binds a CD98 and a second antigen binding region which binds a brain antigen, such as beta-secretase 1 (BACE1), tau, and the other brain antigens disclosed herein. Techniques for making multi-specific antibodies include, but are not limited to, recombinant co-expression of two immunoglobulin heavy chain-light chain pairs having different specificities (see Milstein and Cuello, Nature 305: 537, 1983), WO 93/08829, and Traunecker et al, EMBO J. 10: 3655, 1991), and “knob-in-hole” engineering (see, e.g., U.S. Pat. No. 5,731,168). Multi-specific antibodies can also be made by engineering electrostatic steering effects (WO 2009/089004A1); cross-linking two or more antibodies or fragments (see, e.g., U.S. Pat. No. 4,676,980, and Brennan et al, Science, 229: 81, 1985); using leucine zippers (see, e.g., Kostelny et al, J. Immunol., 148(5): 1547-1553, 1992)); using “diabody” technology (see, e.g., Hollinger et al, Proc. Natl. Acad. Sci. USA, 90:6444-6448, 1993)); using single-chain Fv (sFv) dimers (see, e.g. Gruber et al, J. Immunol, 152:5368 (1994)); and preparing trispecific antibodies as described, e.g., in Tutt et al. J. Immunol. 147: 60, 1991. A multi-specific antibody of the application also encompasses antibodies having three or more functional antigen binding sites, including “Octopus antibodies” or “dual-variable domain immunoglobulins” (DVDs) (see, e.g. US 2006/0025576A1, and Wu et al. Nature Biotechnology, 25(11):1290-7, 2007). A multi-specific antibody of the application also encompasses a “Dual Acting Fab” or “DAF” comprising an antigen binding region that binds to CD98 as well as the brain antigen (e.g. BACE1 or Tau) (see, US 2008/0069820, for example). In one embodiment, the antibody is an antibody fragment, various such fragments being disclosed herein.

In one embodiment, a multi-specific antibody of the application is a fusion construct comprising an anti-CD98 antibody or antigen-binding fragment thereof of the application covalently linked (or fused) to a second antibody or antigen binding fragment thereof. Preferably, the second antibody or antigen binding fragment thereof binds to a brain target, such as BACE, tau or other brain antigens, such as those described herein. The anti-CD98 antibody or antigen-binding fragment thereof can be fused to the carboxy- and/or amino-terminus of a light and/or heavy chain of the second antibody or antigen binding fragment thereof, directly or via a linker.

In one embodiment, the anti-CD98 antibody or antigen-binding fragment thereof is fused to the carboxy-terminus of a light chain of the second antibody or antigen binding fragment thereof, directly or via a linker.

In another embodiment, the anti-CD98 antibody or antigen-binding fragment thereof is fused to the amino-terminus of a light chain of the second antibody or antigen binding fragment thereof, directly or via a linker.

In another embodiment, the anti-CD98 antibody or antigen-binding fragment thereof is fused to the carboxy-terminus of a heavy chain of the second antibody or antigen binding fragment thereof, directly or via a linker.

In another embodiment, the anti-CD98 antibody or antigen-binding fragment thereof is fused to the amino-terminus of a heavy chain of the second antibody or antigen binding fragment thereof, directly or via a linker.

In a preferred embodiment, a fusion construct of the application comprises an anti-CD98 antibody or antigen-binding fragment thereof, preferably an anti-huCD98hc VHH or scFv fragment, of the application covalently linked, via a linker, to the carboxy terminus of only one of the two heavy chains of a second antibody or antigen binding fragment thereof that binds to a brain target. Preferably, the linker has the amino acid sequence of SEQ ID NO: 312 or SEQ ID NO: 313.

To facilitate the formation of a heterodimer between the two heavy chains, e.g., one with a fusion of the anti-CD98 antibody or antigen-binding fragment thereof and one without, or one containing the Fc for the anti-CD98 arm and one for the anti-brain target arm, heterodimeric mutations introduced into the Fc of the two heavy chains. Examples of such Fc mutations include, but are not limited to, the Zymework mutations (see, e.g., U.S. Pat. No. 10,457,742) and the “knob in hole” mutations (see, e.g., Ridgway et al., Protein Eng., 9(7): 617-621, 1996). Other heterodimer mutations can also be used in the invention. In some embodiment, a modified CH3 as described herein is used to facilitate the formation of a heterodimer between the two heavy chains.

In addition to the heterodimeric mutations, other mutations can also be introduced. In some embodiment, the Fc region of the fusion construct or bispecific antibody further comprises one or more mutations that alter (increase or diminish), preferably eliminate ADCC/CDC (such as the AAS mutations described herein), and/or one or more mutations that alter (increase or diminish), preferably increase, the binding of the fusion construct or bispecific antibody to FcRn (such as the YTE mutations described herein). In some embodiment, one or more cysteine residues in the fusion construct or bispecific antibody are substituted with other amino acids, such as serine.

In certain embodiments, a fusion construct of the application comprises:
(1) a first heavy chain having an amino acid sequence that is at least 80%, such as at least 85%, 90%, 95% or 100%, identical to an amino acid sequence selected from the group consisting of SEQ ID NOs: 13, 16, 19, 22, 25, 32, 35, 38, 41, 44, 51, 54, 57, 60, 63, 73, 76, 79, 82, 85, 95, 98, 101, 104, 107, 119, 129, 139, 146, 156, 166, 176, 186, 200, 210, and 219;
(2) two light chains each independently having an amino acid sequence that is at least 80%, such as at least 85%, 90%, 95% or 100%, identical to an amino acid sequence selected from the group consisting of SEQ ID NOs: 14, 17, 20, 23, 26, 33, 36, 42, 45, 52, 55, 58, 61, 64, 74, 77, 80, 83, 86, 96, 99, 102, 105, 108, 120, 130, 140, 147, 157, 167, 177, 187, 201, 211, and 220, respectively; and
(3) a second heavy chain having an amino acid sequence that is at least 80%, such as at least 85%, 90%, 95% or 100%, identical to an amino acid sequence selected from the group consisting of SEQ ID NOs: 15, 18, 21, 24, 27, 34, 37, 43, 46, 53, 56, 59, 62, 65, 75, 78, 81, 84, 87, 97, 100, 103, 106, 109, 121, 131, 141, 148, 158, 168, 178, 188, 202, 212, and 221, respectively.

A conjugate, such as a multi-specific antibody or fusion construct, of the application can be produced by any of a number of techniques known in the art in view of the present disclosure. For example, it can be expressed from a recombinant host cells, wherein expression vector(s) encoding the heavy and light chains of the fusion construct or multi-specific antibody is (are) transfected into a host cell by standard techniques. The host cells can be prokaryotic or eukaryotic host cells.

In an exemplary system, one or more recombinant expression vectors encoding the heterodimeric two heavy chains and the light chains of a fusion construct of the application is/are introduced into host cells by transfection or electroporation. The selected transformant host cells are cultured to allow for expression of the heavy and light chains under conditions sufficient to produce the fusion construct, and the fusion construct is recovered from the culture medium.

Standard molecular biology techniques are used to prepare the recombinant expression vector, transfect the host cells, select for transformants, culture the host cells and recover the protein construct from the culture medium.

The application provides an isolated nucleic acid encoding the amino acid sequence of an anti-CD98 antibody or antigen binding fragment thereof alone or as part of a fusion construct or multispecific antibody in any of the embodiments described herein or any of the claims. The isolated nucleic acid can be part of a vector, preferably an expression vector.

In another aspect, the application relates to a host cell transformed with the vector disclosed herein. In an embodiment, the host cell is a prokaryotic cell, for example, E. coli. In another embodiment, the host cell is a eukaryotic cell, for example, a protist cell, an animal cell, a plant cell, or a fungal cell. In an embodiment, the host cell is a mammalian cell including, but not limited to, CHO, COS, NSO, SP2, PER.C6, or a fungal cell, such as Saccharomyces cerevisiae, or an insect cell, such as Sf9.

Pharmaceutical Composition and Related Methods

The invention also relates to pharmaceutical compositions, methods of preparation and methods for use thereof.

In another general aspect, the invention relates to a pharmaceutical composition, comprising an anti-CD98 antibody or antigen binding fragment thereof or a conjugate thereof of the invention and a pharmaceutically acceptable carrier. The anti-CD98 antibody or antigen binding fragment thereof or conjugate (such as a multi-specific antibody or fusion construct) of the invention is also useful in the manufacture of a medicament for therapeutic applications mentioned herein. The pharmaceutically acceptable carrier can be any suitable excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application.

Accordingly, in one embodiment, the application relates to a method of transporting a therapeutic or diagnostic agent across the blood-brain barrier (BBB) comprising exposing an anti-CD98 antibody or antigen binding fragment thereof coupled to the therapeutic or diagnostic agent to the blood-brain barrier such that the antibody or antigen binding fragment thereof transports the agent coupled thereto across the blood-brain barrier. In one embodiment, the agent is a neurological disorder drug. In another embodiment, the agent is an imaging agent or an agent for detecting a neurological disorder. Preferably, the anti-CD98 antibody or antigen binding fragment thereof or conjugate thereof does not interfere with amino acid transport. The antibody specifically binds to CD98 in such a manner that it does not interfere with amino acid transport. In some embodiments, the BBB is in a mammal, preferably a primate, such as a human, more preferably a human having a neurological disorder. In one embodiment, the neurological disorder is selected from the group consisting of Alzheimer's disease (AD), stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, and traumatic brain injury.

In one embodiment, an anti-CD98 antibody or antigen binding fragment thereof, or a conjugate thereof of the application, is used to detect a neurological disorder before the onset of symptoms and/or to assess the severity or duration of the disease or disorder. The antibody, antigen binding fragment or conjugate thereof permits detection and/or imaging of the neurological disorder, including imaging by radiography, tomography, or magnetic resonance imaging (MRI).

In another embodiment, an anti-CD98 antibody or antigen binding fragment thereof, or a conjugate thereof, is used in treating a neurological disorder (e.g., Alzheimer's disease), comprising administering to a subject in need of the treatment an effective amount of anti-CD98 antibody or antigen binding fragment thereof, or a conjugate thereof. In some embodiments, the method further comprises administering to the subject an effective amount of at least one additional therapeutic agent.

In another embodiment, the application relates to the use of an anti-CD98 antibody or antigen binding fragment or conjugate thereof of the application in the manufacture or preparation of a medicament. In one embodiment, the medicament is for treatment of neurological disease or disorder. In a further embodiment, the medicament is for use in a method of treating neurological disease or disorder comprising administering to an individual having neurological disease or disorder an effective amount of the medicament.

Another general aspect of the application relates to a method of inducing antibody dependent phagocytosis (ADP) without stimulating secretion of a pro-inflammatory cytokine in a subject in need thereof, comprising administering to the subject a complex comprising a therapeutic antibody or antigen binding fragment thereof coupled to, preferably covalently conjugated to, the antigen-binding fragment thereof of an anti-CD98 antibody binding fragment according to an embodiment of the application, wherein the therapeutic antibody or antigen binding fragment thereof does not have effector function. For example, the therapeutic antibody or antigen binding fragment thereof can comprise one or more amino acid modifications that reduces or eliminates the effector function, such as the ADCC or CDC, such as mutations that reduce or abolish the binding to Fc gamma receptor. Such mutations can be at positions L234, L235, D270, N297, E318, K320, K322, P331, and P329, such as one, two or three mutations of L234A, L235A and P331S, wherein the numbering of amino acid residues is according to the EU index as set forth in Kabat. In one embodiment, the therapeutic antibody or antigen binding fragment thereof binds specifically to tau aggregates.

In some embodiments, the method further comprises administering to the subject an effective amount of at least one additional therapeutic agent. In certain embodiments, an additional therapeutic agent is a therapeutic agent effective to treat the same or a different neurological disorder as the anti-CD98 antibody or antigen binding fragment or conjugate thereof is being employed to treat. Exemplary additional therapeutic agents include, but are not limited to: the various neurological drugs described above, cholinesterase inhibitors (such as donepezil, galantamine, rovastigmine, and tacrine), NMDA receptor antagonists (such as memantine), amyloid beta peptide aggregation inhibitors, antioxidants, γ-secretase modulators, nerve growth factor (NGF) mimics or NGF gene therapy, PPARy agonists, HMS-CoA reductase inhibitors (statins), ampakines, calcium channel blockers, GABA receptor antagonists, glycogen synthase kinase inhibitors, intravenous immunoglobulin, muscarinic receptor agonists, nicrotinic receptor modulators, active or passive amyloid beta peptide immunization, phosphodiesterase inhibitors, serotonin receptor antagonists and anti-amyloid beta peptide antibodies. In certain embodiments, the at least one additional therapeutic agent is selected for its ability to mitigate one or more side effects of the neurological drug. The additional therapeutic agent can be administered in the same or separate formulations and administered together or separately with the anti-CD98 antibody or antigen binding fragment or conjugate thereof. The anti-CD98 antibody or antigen binding fragment or conjugate of the application can be administered prior to, simultaneously with, and/or following, the administration of the additional therapeutic agent and/or adjuvant. The anti-CD98 antibody or antigen binding fragment or conjugate thereof of the application can also be used in combination with other interventional therapies such as, but not limited to, radiation therapy, behavioral therapy, or other therapies known in the art and appropriate for the neurological disorder to be treated or prevented.

The anti-CD98 antibody or antigen binding fragment or conjugate thereof of the application (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration, depending in part on whether the administration is brief or chronic. Various dosing schedules including but not limited to single or multiple administrations over various time-points, bolus administration, and pulse infusion are contemplated herein.

For the prevention or treatment of a disease, the appropriate dosage of an anti-CD98 antibody or antigen binding fragment or conjugate thereof of the application (when used alone or in combination with one or more other additional therapeutic agents) will depend on various factors, such as the type of disease to be treated, the type of antibody or conjugate, the severity and course of the disease, whether the antibody, antigen binding fragment or conjugate thereof is administered for preventive or therapeutic purposes, previous therapy, the patient's clinical history and response to the antibody, the physiological state of the subject (including, e.g., age, body weight, health), and the discretion of the attending physician. Treatment dosages are optimally titrated to optimize safety and efficacy. The antibody, antigen binding fragment or conjugate thereof is suitably administered to the patient at one time or over a series of treatments.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

In another aspect, the application relates to an article of manufacture (such as a kit) containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers can be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody, antigen binding fragment thereof or a conjugate of the application. The label or package insert indicates that the composition is used for treating the condition of choice. Moreover, the article of manufacture can include (a) a first container with a composition contained therein, wherein the composition comprises an antibody, antigen binding fragment thereof or a conjugate of the application; and (b) a second container with a composition contained therein, wherein the composition comprises a further cytotoxic or otherwise therapeutic agent. The article of manufacture in this embodiment of the invention can further include a package insert indicating that the compositions can be used to treat a particular condition. Optionally, the article of manufacture can further comprise a second (or third) container comprising a pharmaceutically acceptable buffer, such as bacteriostatic water for injection (BWFI), phosphate-buffered saline, Ringer's solution and dextrose solution. It can further include other materials desirable from a commercial and user standpoint, including other buffers, diluents, filters, needles, and syringes.

### EMBODIMENTS

The invention provides also the following non-limiting embodiments.

1. An anti-CD98 antibody or antigen-binding fragment thereof for delivering an agent to the brain of a subject in need thereof, wherein the anti-CD98 antibody or antigen-binding fragment thereof binds to a CD98, preferably human CD98 heavy chain (huCD98hc), with a dissociation constant KD at least 1 nM at a neutral pH and an off-rate constant kd of at least 10⁻⁴ sec⁻¹ at an acidic pH, preferably pH 5.

1a. The anti-CD98 antibody or antigen-binding fragment thereof of embodiment 1, having a dissociation constant KD of 1 nM to 500 nM, such as 1 nM, 10 nM, 50 nM, 100 nM, 200 nM, 300 nM, 400 nM, 500 nM, or any value in between, at the neutral pH.

1b. The anti-CD98 antibody or antigen-binding fragment thereof of embodiment 1 or 1a, having an off-rate constant kd of 10⁻⁴ sec⁻¹ to 10⁻¹ sec⁻¹, such as 10⁻⁴, 10⁻³, 10⁻², 10⁻¹ sec⁻¹ or any value in between, at the acidic pH.

2. The anti-CD98 antibody or antigen-binding fragment thereof of any one of embodiments 1 to 1b, having an off-rate constant kd of 2×10⁻² to 2×10⁻⁴ sec⁻¹, preferably 8×10⁻³ sec⁻¹, at the neutral pH.

2a. The anti-CD98 antibody or antigen-binding fragment thereof of embodiment 2, wherein the off-rate constant kd at the neutral pH is 2×10⁻² to 2×10⁻⁴ sec⁻¹, such as 2×10⁻², 1×10⁻², 9×10⁻³, 8×10⁻³, 7×10⁻³, 6×10⁻³, 5×10⁻³, 4×10⁻³, 3×10⁻³, 2×10⁻³, 1×10⁻³, 9×10⁻⁴, 8×10⁻⁴, 7×10⁻⁴, 6×10⁻⁴, 5×10⁻⁴, 4×10⁻⁴, 3×10⁻⁴, 2×10⁻⁴ sec⁻¹, or any value in between.

3. The anti-CD98 antibody or antigen-binding fragment thereof of any one of embodiments 1 to 2a, comprising
(1) a heavy chain variable region comprising heavy chain complementarity determining regions (HCDRs) HCDR1, HCDR2 and HCDR3, and a light chain variable region comprising light chain complementarity determining regions (LCDRs) LCDR1, LCDR2 and LCDR3, wherein the HCDR1, HCDR2, HCDR3, LCDR1, LCDR2 and LCDR3 have the amino acid sequences of:
   i. SEQ ID NOs: 7, 8, 9, 10, 11 and 12, respectively;
   ii. SEQ ID NOs: 67, 68, 69, 70, 71 and 72, respectively;
   iii. SEQ ID NOs: 89, 90, 91, 92, 93 and 94, respectively;
   iv. SEQ ID NOs: 113, 114, 115, 116, 117 and 118, respectively;
   v. SEQ ID NOs: 123, 124, 125, 126, 127 and 128, respectively;
   vi. SEQ ID NOs: 133, 134, 135, 136, 137 and 138, respectively;
   vii. SEQ ID NOs: 150, 151, 152, 153, 154 and 155, respectively;
   viii. SEQ ID NOs: 160, 161, 162, 163, 164 and 165, respectively;
   ix. SEQ ID NOs: 170, 171, 172, 173, 174 and 175, respectively;
   x. SEQ ID NOs: 180, 181, 182, 183, 184 and 185, respectively; or
(2) a single variable domain on a heavy chain (VHH) comprising heavy chain complementarity determining regions (HCDRs) HCDR1, HCDR2 and HCDR3 having the amino acid sequences of:
   i. SEQ ID NOs: 29, 30 and 31, respectively;
   ii. SEQ ID NOs: 48, 49 and 50, respectively; or
   iii. SEQ ID NOs: 143, 144 and 145, respectively.

4. The antibody or antigen-binding fragment thereof of embodiment 3, being a VHH fragment comprising an amino acid sequence having at least 80%, such as at least 85%, 90%, 95% or 100%, sequence identity to SEQ ID NO: 28, SEQ ID NO: 47, or SEQ ID NO: 142.

4a. The antibody or antigen-binding fragment thereof of embodiment 3, wherein the VHH fragment comprising the amino acid sequence of SEQ ID NO: 28, SEQ ID NO: 47, or SEQ ID NO: 142.

5. The antibody or antigen-binding fragment thereof of embodiment 3, being a single-chain variable fragment (scFv) comprising the heavy chain variable region (VH) covalently linked to the light chain variable region (VL) via a linker, such as a peptide linker having about 10 to about 25 amino acids in length.

5a. The antibody or antigen-binding fragment thereof of embodiment 5, wherein the VH is linked to the amino-terminus of the VL via the linker in the scFv.

5b. The antibody or antigen-binding fragment thereof of embodiment 5, wherein the VH is linked to the carboxy-terminus of the VL via the linker in the scFv.

5c. The antibody or antigen-binding fragment thereof of embodiment 5a or 5b, wherein the linker comprises one or more of Gly and Ser, with one or more interspersed Glu, Thr and Lys residues, preferably the linker has the amino acid sequence of SEQ ID NO: 189.

5d. The antibody or antigen-binding fragment thereof of embodiment 5c, wherein the scFv comprises the HCDR1, HCDR2, HCDR3, LCDR1, LCDR2 and LCDR3 having the amino acid sequences of SEQ ID NOs: 279, 280, 281, 282, 283 and 284, respectively, or SEQ ID NOs: 292, 293, 294, 295, 296, and 297, respectively;

5e. The antibody or antigen-binding fragment thereof of embodiment 5c or 5d, wherein the scFv comprises an amino acid sequence having at least 80%, such as at least 85%, 90%, 95% or 100%, sequence identity to the amino acid sequences of SEQ ID NO: 6, SEQ ID NO: 66, SEQ ID NO: 88, SEQ ID NO: 112, SEQ ID NO: 122, SEQ ID NO: 132, SEQ ID NO: 149, SEQ ID NO: 159, SEQ ID NO: 169, SEQ ID NO: 179, SEQ ID NO: 193, or SEQ ID NO: 203.

5f. The antibody or antigen-binding fragment thereof of embodiment 5e, wherein the scFv comprises the amino acid sequence of SEQ ID NO: 6, SEQ ID NO: 66, SEQ ID NO: 88, SEQ ID NO: 112, SEQ ID NO: 122, SEQ ID NO: 132, SEQ ID NO: 149, SEQ ID NO: 159, SEQ ID NO: 169, SEQ ID NO: 179, SEQ ID NO: 193, or SEQ ID NO: 203.

5g. The antibody or antigen-binding fragment thereof of embodiment 5e, wherein the scFv comprises the amino acid sequence of SEQ ID NO: 278, 291, 162 or 218.

5h. An antibody or antigen-binding fragment thereof that binds to the same epitope of the antibody or antigen-binding fragment thereof of any one of embodiments 1 to 5g.

5i. An antibody or antigen-binding fragment thereof that competes with the antibody or antigen-binding fragment thereof of any one of embodiments 1 to 5g in binding to the CD98.

5j. The antibody or antigen-binding fragment thereof of any one of embodiments 3 to 5i, binding to human CD98 with a dissociation constant KD of 1 to 500 nM, such as 1 nM, 10 nM, 50 nM, 100 nM, 200 nM, 300 nM, 400 nM, 500 nM, or any value in between, at pH7.4

5k. The antibody or antigen-binding fragment thereof of any one of embodiments 1 to 5j, binding to human CD98 with an off-rate constant kd of 10⁻⁴ to 10⁻¹ sec⁻¹, such as 10⁻⁴, 10⁻¹, 10⁻², 10⁻¹ sec⁻¹ or any value in between, at pH5.

6. A complex comprising the antibody or antigen-binding fragment thereof of any one of embodiments 1 to 5k coupled to a therapeutic or diagnostic agent.

6a. The complex of embodiment 6, wherein the antibody or antigen-binding fragment thereof is coupled to the therapeutic or diagnostic agent noncovalently.

6b. The complex of embodiment 6, wherein the antibody or antigen-binding fragment thereof is coupled to the therapeutic or diagnostic agent covalently to form a conjugate.

6c. The complex of embodiment 6, wherein the antibody or antigen-binding fragment thereof is covalently linked to the therapeutic or diagnostic agent via a linker.

6d. The complex of embodiment 6c, wherein the linker is a non-peptide linker, such as polyethylene glycol, polypropylene glycol, copolymer of ethylene glycol and propylene glycol, polyoxyethylated polyol, polyvinyl alcohol, polysaccharides, dextran, polyvinyl ether, biodegradable polymer, polymerized lipid, chitins, and hyaluronic acid, or derivatives thereof, or combinations thereof.

6e. The complex of embodiment 6c, wherein the linker is a peptide linker, such as a peptide chain consisting of 1 to 50 amino acids linked by peptide bonds or a derivative thereof.

6f. The complex of any one of embodiments 6 to 6e, wherein the antibody or antigen-binding fragment thereof is coupled to the diagnostic agent for detecting a neurological disorder, preferably, the diagnostic agent is an agent for positron emission tomography (PET), or an agent for IDK.

6g. The complex of any one of embodiments 6 to 6e, wherein the antibody or antigen-binding fragment thereof is coupled to a therapeutic agent, preferably a neurological disorder drug.

6h. The complex of embodiment 6g, wherein the neurological disorder drug is selected from the group consisting of small molecule compounds, antibodies, peptides, proteins, natural ligands of one or more CNS target(s), modified versions of natural ligands of one or more CNS target(s), aptamers, inhibitory nucleic acids (i.e., small inhibitory RNAs (siRNA) and short hairpin RNAs (shRNA)), ribozymes, and active fragments of the foregoing.

6i. The complex of embodiment 6g, wherein the neurological disorder drug is selected from the group consisting of antibodies, aptamers, proteins, peptides, inhibitory nucleic acids and small molecules and active fragments of any of the foregoing that either are themselves or specifically recognize and/or act upon (i.e., inhibit, activate, or detect) a CNS antigen or target molecule such as, but not limited to, amyloid precursor protein or portions thereof, amyloid beta, beta-secretase, gamma-secretase, tau, alpha-synuclein, parkin, huntingtin, DR6, presenilin, ApoE, glioma or other CNS cancer markers, and neurotrophins Non-limiting examples of neurological disorder drugs and the corresponding disorders they may be used to treat: Brain-derived neurotrophic factor (BDNF), Chronic brain injury (Neurogenesis), Fibroblast growth factor 2 (FGF-2), Anti-Epidermal Growth Factor Receptor Brain cancer, (EGFR)-antibody, Glial cell-line derived neural factor Parkinson's disease, (GDNF), Brain-derived neurotrophic factor (BDNF) Amyotrophic lateral sclerosis, depression, Lysosomal enzyme Lysosomal storage disorders of the brain, Ciliary neurotrophic factor (CNTF) Amyotrophic lateral sclerosis, Neuregulin-1 Schizophrenia, Anti-HER2 antibody (e.g. trastuzumab) Brain metastasis from HER2-positive cancer.

7. The complex of embodiment 6, being a multi-specific antibody comprising a first antigen binding region which binds a CD98 and a second antigen binding region which binds a brain antigen (or brain target), wherein the first antigen binding region comprises the antigen-binding fragment thereof of any one of embodiments 1 to 5k.

7a. The multi-specific antibody of embodiment 7, wherein the brain target is selected from the group consisting of beta-secretase 1 (BACE1), amyloid beta (Abeta), epidermal growth factor receptor (EGFR), human epidermal growth factor receptor 2 (HER2), Tau, apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), and caspase 6.

7b. The multi-specific antibody of embodiment 7a, wherein the second antigen binding region binds a BACE1 or Tau.

7c. The multi-specific antibody of any one of embodiments 7 to 7b, wherein the first antigen binding region is covalently linked to a first Fc, and the second antigen binding region is covalently linked to a second Fc.

7d. The multi-specific antibody of embodiment 7c, wherein the first Fc is different from the second Fc in one or more amino acid residues to facilitate the formation of a heterodimer between the first Fc and the second Fc.

8. The multi-specific antibody of any one of embodiments 7 to 7b, being a fusion construct comprising the antibody or antigen-binding fragment thereof of any one of embodiments 1 to 5k covalently linked a second antibody or antigen binding fragment thereof that binds the brain antigen (or brain target).

8a. The fusion construct of embodiment 8, wherein the antibody or antigen-binding fragment thereof of any one of embodiments 1 to 5k is covalently linked, preferably via a linker, to the amino-terminus of a heavy chain of the second antibody or antigen binding fragment thereof.

8b. The fusion construct of embodiment 8, wherein the antibody or antigen-binding fragment thereof of any one of embodiments 1 to 5k is covalently linked, preferably via a linker, to the amino-terminus of a light chain of the second antibody or antigen binding fragment thereof.

8c. The fusion construct of embodiment 8, wherein the antibody or antigen-binding fragment thereof of any one of embodiments 1 to 5k is covalently linked, preferably via a linker, to the carboxy-terminus of a light chain of the second antibody or antigen binding fragment thereof.

8d. The fusion construct of embodiment 8, wherein the antibody or antigen-binding fragment thereof of any one of embodiments 1 to 5k is covalently linked, preferably via a linker, to the carboxy-terminus of a heavy chain of the second antibody or antigen binding fragment thereof.

9. The fusion construct of embodiment 8d, wherein the antibody or antigen-binding fragment thereof any one of embodiments 1 to 5k is covalently linked, via a linker, to the carboxy terminus of only one of the two heavy chains of the second antibody or antigen binding fragment.

9a. The fusion construct of any one of embodiments 8a to 9, wherein the linker is a peptide linker comprising one or more of Gly and Ser, preferably the linker has the amino acid sequence of SEQ ID NO: 190 or SEQ ID NO: 191.

9b. The fusion construct of any one of embodiments 8 to 9a, wherein the second antibody or antigen binding fragment thereof comprise a first Fc in its first heavy chain and a second Fc in its second heavy chain, and the first Fc is different from the second Fc in one or more amino acid residues to facilitate the formation of a heterodimer between the first Fc and the second Fc.

9c. The multi-specific antibody of embodiment 7d or the fusion construct of embodiment 9b, wherein the first Fc contains one or more “knob” mutations and the second Fc contains more or more corresponding “hole” mutations, or vice versa (see, e.g., U.S. Pat. No. 5,731,168, Ridgway et al., Protein Eng., 9(7): 617-621, 1996, for the “knob-in-hole” mutations, which are incorporated herein by reference in its entirety), preferably a Knob mutation of T366W and a hole mutation of T366S, L368A or Y407V.

9d. The multi-specific antibody of embodiment 7d or the fusion construct of embodiment 9b, wherein each of the first Fc and the second Fc comprises a modified heterodimeric CH3 domain as compared to a wild-type CH3 domain polypeptide, preferably, the modified heterodimeric CH3 domain comprises one or more mutations as described in U.S. Pat. No. 10,457,742.

9e. The multi-specific antibody or fusion construct of embodiment 9d, wherein the modified heterodimeric CH3 domain of the first Fc comprises amino acid modifications at positions T350, L351, F405, and Y407, and the modified heterodimeric CH3 domain of the second Fc comprises amino acid modifications at positions T350, T366, K392 and T394.

9f. The multi-specific antibody or fusion construct of embodiment 9e, wherein the amino acid modification at position T350 is T350V, T350I, T350L or T350M; the amino acid modification at position L351 is L351 Y; the amino acid modification at position F405 is F405A, F405V, F405T or F405S; the amino acid modification at position Y407 is Y407V, Y407A or Y407I; the amino acid modification at position T366 is T366L, T366I, T366V or T366M, the amino acid modification at position K392 is K392F, K392L or K392M, and the amino acid modification at position T394 is T394W.

9g. The multi-specific antibody or fusion construct of embodiment 9e, wherein the modified heterodimeric CH3 domain of the first Fc comprises mutations T350V, L351Y, F405A and Y407V, and the modified heterodimeric CH3 domain of the second Fc comprises mutations T350V, T366L, K392L and T394W, or vice versa.

10. The multi-specific antibody or fusion construct of any one of embodiments 7 to 9g, wherein the Fc region of the multi-specific antibody or fusion construct further comprises substitutions that alter (increase or diminish), preferably increase, the binding of the second antibody or antigen binding fragment thereof to neonatal Fc receptor (FcRn).

10a. The multi-specific antibody or fusion construct of embodiment 10, wherein the second antibody or antigen binding fragment thereof comprises one or more mutations in the Fc domain that enhance binding of the fusion to the neonatal Fc receptor (RcRn).

10b. The multi-specific antibody or fusion construct of embodiment 10 or 10a, wherein the one or more mutations enhance the binding at an acidic pH.

10c. The multi-specific antibody or fusion construct of embodiment 10b, wherein the Fc of the second antibody has the M252Y/S254T/T256E (YTE) mutations, wherein the numbering of amino acid residues is according to the EU index as set forth in Kabat.

11. The multi-specific antibody or fusion construct of any one of embodiments 7-10c, wherein the Fc region of the multi-specific antibody or fusion construct further comprises substitutions that alter (increase or diminish), preferably reduces or eliminates the effector function.

11a. The multi-specific antibody or fusion construct of embodiment 11, wherein the second antibody or antigen binding fragment thereof comprises one or more mutations in the Fc domain that reduce or eliminate the effector function, such antibody dependent cellular cytotoxicity (ADCC) or complement dependent cytotoxicity (CDC).

11b. The multi-specific antibody or fusion construct of embodiment 11a, wherein the Fc of the second antibody has one or more amino acid modifications at positions L234, L235, D270, N297, E318, K320, K322, P331, and P329, wherein the numbering of amino acid residues is according to the EU index as set forth in Kabat.

11c. The multi-specific antibody or fusion construct of embodiment 11b, wherein the Fc of the second antibody has one, two or three mutations of L234A, L235A and P331S (the AAS mutations).

12. The multi-specific antibody or fusion construct of any one of embodiments 7 to 11c, wherein the first antigen binding region or the antibody or antigen-binding fragment thereof does not contain cysteine.

13. The multi-specific antibody or fusion construct of embodiments 7 to 12, wherein the second antigen binding region or the second antibody or antigen binding fragment thereof that binds Tau, preferably comprising the HCDR1, HCDR2, HCDR3, LCDR1, LCDR2 and LCDR3 having the amino acid sequences of SEQ ID NOs: 213-218, respectively, preferably, the second antibody is a monoclonal antibody comprising a heavy chain having the amino acid sequence of SEQ ID NO: 110 and a light chain having the amino acid sequence of SEQ ID NO: 111.

14. The fusion construct of embodiment 9, comprising:
(1) a first heavy chain having an amino acid sequence that is at least 80%, such as at least 85%, 90%, 95% or 100%, identical to an amino acid sequence selected from the group consisting of SEQ ID NOs: 13, 16, 19, 22, 25, 32, 35, 38, 41, 44, 51, 54, 57, 60, 63, 73, 76, 79, 82, 85, 95, 98, 101, 104, 107, 119, 129, 139, 146, 156, 166, 176, 186, 200, 210, and 219;
(2) two light chains each independently having an amino acid sequence that is at least 80%, such as at least 85%, 90%, 95% or 100%, identical to an amino acid sequence selected from the group consisting of SEQ ID NOs: 14, 17, 20, 23, 26, 33, 36, 42, 45, 52, 55, 58, 61, 64, 74, 77, 80, 83, 86, 96, 99, 102, 105, 108, 120, 130, 140, 147, 157, 167, 177, 187, 201, 211, and 220, respectively; and
(3) a second heavy chain having an amino acid sequence that is at least 80%, such as at least 85%, 90%, 95% or 100%, identical to an amino acid sequence selected from the group consisting of SEQ ID NOs: 15, 18, 21, 24, 27, 34, 37, 43, 46, 53, 56, 59, 62, 65, 75, 78, 81, 84, 87, 97, 100, 103, 106, 109, 121, 131, 141, 148, 158, 168, 178, 188, 202, 212, and 221, respectively.

14a. The fusion construct of embodiment 14, wherein the two light chains have an identical amino acid sequence.

14b. The fusion construct of embodiment 14, wherein the two light chains have different amino acid sequences.

14c. The fusion construct of embodiment 14, wherein:
(1) the first heavy chain has the amino acid sequence selected from the group consisting of SEQ ID NOs: 13, 16, 19, 22, 25, 32, 35, 38, 41, 44, 51, 54, 57, 60, 63, 73, 76, 79, 82, 85, 95, 98, 101, 104, 107, 119, 129, 139, 146, 156, 166, 176, 186, 200, 210, and 219;
(2) the two light chains each have the amino acid sequence selected from the group consisting of SEQ ID NOs: 14, 17, 20, 23, 26, 33, 36, 42, 45, 52, 55, 58, 61, 64, 74, 77, 80, 83, 86, 96, 99, 102, 105, 108, 120, 130, 140, 147, 157, 167, 177, 187, 201, 211, and 220, respectively; and
(3) the second heavy chain has the amino acid sequence selected from the group consisting of SEQ ID NOs: 15, 18, 21, 24, 27, 34, 37, 43, 46, 53, 56, 59, 62, 65, 75, 78, 81, 84, 87, 97, 100, 103, 106, 109, 121, 131, 141, 148, 158, 168, 178, 188, 202, 212, and 221, respectively.

15. An isolated nucleic acid encoding the antibody or antigen-binding fragment of any one of embodiments 1-5k, or the fusion construct of any one of embodiments 7-14c.

16. A vector comprising the isolated nucleic acid of claim 15.

17. A host cell comprising the nucleic acid of embodiment 15 or the vector of claim 16.

18. A method of producing the antibody or antigen-binding fragment of any one of embodiments 1-5k, or the fusion construct of any one of embodiments 7-14c, comprising culturing a cell comprising a nucleic acid encoding the antibody or antigen-binding fragment or the fusion construct under conditions to produce the antibody or antigen-binding fragment or the fusion construct, and recovering the antibody or antigen-binding fragment or the fusion construct from the cell or cell culture.

19. A pharmaceutical composition comprising the antibody or antigen-binding fragment of any one of embodiments 1-5k, the complex of any one of embodiments 6-6i, or the multi-specific antibody or fusion construct of any one of embodiments 7-14c, and a pharmaceutically acceptable carrier.

20. A method of treating or detecting a neurological disorder in a subject in need thereof, comprising administering to the subject an effective amount of the antibody or antigen-binding fragment of any one of embodiments 1-5k, the complex of any one of embodiments 6-6i, or the multi-specific antibody or fusion construct of any one of embodiments 7-14c, or the pharmaceutical composition of embodiment 19.

21. A method of increasing delivery of a therapeutic or diagnostic agent to the brain of a subject in need thereof, comprising administering to the subject a conjugate comprising the therapeutic or diagnostic agent coupled to the antibody or antigen-binding fragment thereof of any one of embodiments 1-5k.

22. A method of transporting a therapeutic or diagnostic agent across the blood-brain barrier (BBB) comprising exposing an anti-CD98 antibody or antigen binding fragment thereof of any one of embodiments 1-5k coupled to the therapeutic or diagnostic agent to the blood-brain barrier such that the antibody or antigen binding fragment thereof transports the agent coupled thereto across the blood-brain barrier.

23. A method of delivering a therapeutic or diagnostic agent across the blood-brain barrier (BBB) of a subject in need thereof, comprising administering to the subject a complex comprising the therapeutic or diagnostic agent coupled to, preferably covalently conjugated to, the antibody or antigen-binding fragment thereof of any one of embodiments 1 to 5.

24. A method of inducing antibody dependent phagocytosis (ADP) without stimulating secretion of a pro-inflammatory cytokine in a subject in need thereof, comprising administering to the subject a complex comprising a therapeutic antibody or antigen binding fragment thereof coupled to, preferably covalently conjugated to, the antigen-binding fragment thereof of any one of embodiments 1 to 5, wherein the therapeutic antibody or antigen binding fragment thereof comprises one or more mutations in the Fc domain that reduce or eliminate the effector function, such antibody dependent cellular cytotoxicity (ADCC) or complement dependent cytotoxicity (CDC).

24a. The method of embodiment 24, wherein the therapeutic antibody or antigen binding fragment thereof comprises one or more amino acid modifications at positions L234, L235, D270, N297, E318, K320, K322, P331, and P329, wherein the numbering of amino acid residues is according to the EU index as set forth in Kabat.

24b. The method of embodiment 24a, wherein the therapeutic antibody or antigen binding fragment thereof comprises one, two or three mutations of L234A, L235A and P331S.

25. The method of any one of embodiments 20-24b, wherein the subject is in need of a treatment of a neurological disorder, preferably the neurological disorder is selected from the group consisting of neurodegenerative diseases (such as Lewy body disease, postpoliomyelitis syndrome, Shy-Draeger syndrome, olivopontocerebellar atrophy, Parkinson's disease, multiple system atrophy, striatonigral degeneration, spinocerebellar ataxia, spinal muscular atrophy), tauopathies (such as Alzheimer disease and supranuclear palsy), prion diseases (such as bovine spongiform encephalopathy, scrapie, Creutz-feldt-Jakob syndrome, kuru, Gerstmann-Straussler-Scheinker disease, chronic wasting disease, and fatal familial insomnia), bulbar palsy, motor neuron disease, and nervous system heterodegenerative disorders (such as Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (such as Pick's disease, and spinocerebellar ataxia), and cancer of the CNS and/or brain (such as brain metastases resulting from cancer elsewhere in the body).

26. The method of any one of embodiments 20 to 25, wherein the antibody or antigen-binding fragment thereof, the complex, the multispecific antibody, the fusion construct, or the pharmaceutical composition is administered intravenously.

27. The method of any one of embodiments 20 to 26, wherein the therapeutic agent or therapeutic antibody or antigen-binding fragment thereof is a neurological disorder drug.

28. The method of any one of embodiments 20 to 23, wherein the agent is an imaging agent or an agent for detecting a neurological disorder.

29. The method of any one of embodiments 20 to 28, wherein the anti-CD98 antibody or antigen binding fragment thereof, complex, or fusion thereof, does not interfere with amino acid transport.

30. The method of any one of embodiments 20 to 29, wherein the administration reduces Fc-mediated effector function.

31. The method of any one of embodiments 21 to 30, wherein the administration does not induce rapid reticulocyte depletion.

32. The method of embodiment 31, wherein the therapeutic antibody or antigen binding fragment thereof binds specifically to tau aggregates.

33. The method of any one of embodiments 20-32, wherein the subject is a primate, such as a human, more preferably a human having a neurological disorder.

34. The method of embodiment 33, wherein the neurological disorder is selected from the group consisting of Alzheimer's disease (AD), stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, and traumatic brain injury.

The following examples of the invention are to further illustrate the nature of the invention. It should be understood that the following examples do not limit the invention and the scope of the invention is to be determined by the appended claims.

### EXAMPLES

### Example 1

While the blood-brain barrier (BBB) prevents harmful substances from entering the brain and is essential for brain homeostasis, it presents a formidable obstacle for efficiently delivering drugs to the brain. Towards this end, a monoclonal antibody (mAb) brain shuttle platform was developed that penetrates the BBB and results in substantially higher brain concentrations than mAb alone.

### Antibody Generation (OMT Rats and Ablexis Mice)

OMT rats (OmniRat® from Ligand Pharmaceuticals) and Ablexis mice (Ablexis, LLC, San Diego, Calif.) were immunized with human CD98hc (SEQ ID NO: 1), or human CD98hc, cyno CD98hc (SEQ ID NO: 2) and marmoset CD98hc (SEQ ID NO: 3). Two protocols were used for OMT immunization, a short (28 days) and a long protocol (48 days), for immunization of 5 OMT rats for each protocol. Ablexis mice were immunized using repetitive immunizations multiple sites (RIMMS) protocol with Sigma/CFA adjuvant for either 20 or 51 days. Briefly, animals were repeatedly immunized at multiple subcutaneous sites proximal to regional draining lymph nodes. Serum titration by ELISA was done at day 31 for the long protocol and at day 14 for the short protocol using huCD98hc ECD coated plates (OMT) or huCD98hc overexpressing CHO-S cells (Ablexis). Lymph nodes were harvested from sera-positive animals and fused to generate hybridomas or screened as single B-cells.

Hybridomas were first screened by ELISA for binding to huCD98hc coated plates. Hits were then tested in a confirmatory screen against human, cyno (SEQ ID NO: 2), marmoset (SEQ ID NO: 3), mouse (SEQ ID NO: 4), and rat (SEQ ID NO: 5) CD98hc coated ELISA plates. Then a cell-based screening assay was completed using CHO-S cells overexpressing huCD98hc or cyCD98hc, pBECS (primary human brain endothelial cortex cells, endogenous CD98hc expression), and CHO parental cells as a negative cell line. After confirmatory ELISA, 94 hits were selected (62 hits from fusion 1 and 32 hits from fusion 2). In the FACS assay, 31 cell binders could be confirmed, out of which 20 were not confirmed positive in ELISA.

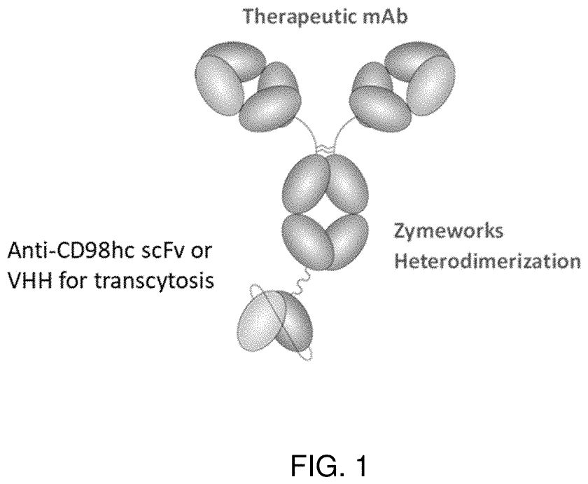

All the clones that were assayed and found positive in at least one assay were re-arrayed (94 clones) and re-measured in ELISA and cell binding (FACS). The data of the first confirmatory screening was confirmed in the second confirmatory screen. mAbs were then assessed for internalization in pBECs. V-region sequencing data was then obtained for mAbs that internalized. The sequencing data was used to construct tripod mAbs (FIG. 1).

### Antibody Generation (Llama)

For the generation of single domain antibodies (VHH) against human CD98hc, two llamas were used for immunizations at Abcore. Antibody titers were determined by ELISA using human CD98hc protein (1 μg/ml). Three bleeds from the two animals were tested and for both animals, both animals showed good early titers.

Phage display was done at Abcore using their standard protocol. Two libraries were made: Library 1 from the second bleed of both animals, and Library 2 from the second and third bleed. Plasmid DNA from 12 random individual clones were sequenced and >80% contained VHH inserts with the correct reading frame. Both phage display libraries were screened with human CD98hc using standard Abcore panning procedures. Three rounds of panning with human CD98hc at 10 μg/ml were done. After panning, 94 individual clones were screened by phage ELISA for specific binding to protease-activated receptor 1 (Par1)N-terminal domain and non-specific binding to BSA (bovine serum albumin). Cross reactivity with cyno, mouse, and rat CD98hc was measured. 94 clones that demonstrated binding were selected for sequence analysis in the heavy and light chain variable regions. Sequences were used to construct tripod mAbs (FIG. 1).

Examples of CD98hc antibodies or antigen binding fragments of the invention are summarized in Table 1:

TABLE 1

| Name | Sequence | HCDR1 | HCDR2 | HCDR3 |
|---|---|---|---|---|
| CD98hc 1 | SEQ ID | SEQ ID NO: 7 | SEQ ID NO: 8 | SEQ ID NO: 9 |
| | NO: 6 | GGSISSGAYYW | IYYSGST | ARAGPKWELWFDY |
| CD98hc 2 | SEQ ID | SEQ ID NO: 29 | SEQ ID NO: 30 | SEQ ID NO: 31 |
| | NO: 28 | GRAFSSNG | ISCSGTST | AADTRHCMGYTKDEGYAY |
| CD98hc 3 | SEQ ID | SEQ ID NO: 48 | SEQ ID NO: 49 | SEQ ID NO: 50 |
| | NO: 47 | GRTISTYG | SCSGTST | AADTRHCMGYTKHGGYDY |
| CD98hc 4 | SEQ ID | SEQ ID NO: 67 | SEQ ID NO: 68 | SEQ ID NO: 69 |
| | NO: 66 | GFTFSSYG | ISYDGSNK | ARAPSSFYFDY |
| CD98hc 5 | SEQ ID | SEQ ID NO: 89 | SEQ ID NO: 90 | SEQ ID NO: 91 |
| | NO: 88 | GGSISSSY | IYNSGST | ARETYYFDY |
| CD98hc 6 | SEQ ID | SEQ ID NO: 113 | SEQ ID NO: 114 | SEQ ID NO: 115 |
| | NO: 112 | GYTFTSYY | INPSGGTT | ARDPNDYGGPKYFPH |
| CD98hc 7 | SEQ ID | SEQ ID NO: 123 | SEQ ID NO: 124 | SEQ ID NO: 125 |
| | NO: 122 | EYTFTSYY | INPSGGST | ARAGYCTINSCFNWFDP |
| CD98hc 8 | SEQ ID | SEQ ID NO: 133 | SEQ ID NO: 134 | SEQ ID NO: 135 |
| | NO: 132 | GYTFTSYY | FNPSGGST | ARGGVGATGYFDY |
| CD98hc 9 | SEQ ID | SEQ ID NO: 143 | SEQ ID NO: 144 | SEQ ID NO: 145 |
| | NO: 142 | GRTFSRYG | ISCSGTST | AADTRHCMGYTKDGGYAY |
| CD98hc 10 | SEQ ID | SEQ ID NO: 150 | SEQ ID NO: 151 | SEQ ID NO: 152 |
| | NO: 149 | GYTFTGYY | INPNSGGS | LRGKTFDP |
| CD98hc 11 | SEQ ID | SEQ ID NO: 160 | SEQ ID NO: 161 | SEQ ID NO: 162 |
| | NO: 159 | GYTFTSYY | NPSGGST | ARAGYCRSTTSCFNWFDP |
| CD98hc 12 | SEQ ID | SEQ ID NO: 170 | SEQ ID NO: 171 | SEQ ID NO: 172 |
| | NO: 169 | GFTLSSSW | INSDGSST | ARWNWGWYFDL |
| CD98hc 13 | SEQ ID | SEQ ID NO: 180 | SEQ ID NO: 181 | SEQ ID NO: 182 |
| | NO: 179 | GFTFSSYW | INSDGSII | VREGRYTGSSLDY |
| CD98hc 14 | SEQ ID | SEQ ID NO: 194 | SEQ ID NO: 195 | SEQ ID NO: 196 |
| | NO: 193 | GFTFSSYG | ISYDGSNK | ARAPSAFYFDY |
| CD98hc 15 | SEQ ID | SEQ ID NO: 204 | SEQ ID NO: 205 | SEQ ID NO: 206 |
| | NO: 203 | GFTFSSYG | ISYDGSNK | ARAPSHFYFDY |
| | Name | LCDR1 | LCDR2 | LCDR3 |
| | CD98hc 1 | SEQ ID NO: 10 | SEQ ID NO: 11 | SEQ ID NO: 12 |
| | | SSDVDNYNY | DVSNRPS | SSFTSSSTWV |
| | CD98hc 2 | | | |
| | CD98hc 3 | | | |
| | CD98hc 4 | SEQ ID NO: 70 | SEQ ID NO: 71 | SEQ ID NO: 72 |
| | | QSVLFSSNNKNY | WAS | QQYYSTPPT |
| | CD98hc 5 | SEQ ID NO: 92 | SEQ ID NO: 93 | SEQ ID NO: 94 |
| | | QNVGIN | SAA | QQYNSYPLT |
| | CD98hc 6 | SEQ ID NO: 116 | SEQ ID NO: 117 | SEQ ID NO: 118 |
| | | QSVLYRSNNKNY | WAS | HQYYSTPLT |
| | CD98hc 7 | SEQ ID NO: 126 | SEQ ID NO: 127 | SEQ ID NO: 128 |
| | | QSVLYASRNKNY | WAS | QYYSTPFT |
| | CD98hc 8 | SEQ ID NO: 136 | SEQ ID NO: 137 | SEQ ID NO: 138 |
| | | QSVLYRSNNKNY | WAS | QQYYGTPFT |
| | CD98hc 9 | | | |
| | CD98hc 10 | SEQ ID NO: 153 | SEQ ID NO: 154 | SEQ ID NO: 155 |
| | | QGISSH | AAS | QQLNNYPLT |
| | CD98hc 11 | SEQ ID NO: 163 | SEQ ID NO: 164 | SEQ ID NO: 165 |
| | | QSVLYNSNNKNY | WAS | QQYYSTPFT |
| | CD98hc 12 | SEQ ID NO: 173 | SEQ ID NO: 174 | SEQ ID NO: 175 |
| | | QSLLHSHGDNY | LGS | MQTLETPYT |
| | CD98hc 13 | SEQ ID NO: 183 | SEQ ID NO: 184 | SEQ ID NO: 185 |
| | | NSNIGAGYD | GNS | QSYDSSLTGWV |
| | CD98hc 14 | SEQ ID NO: 197 | SEQ ID NO: 198 | SEQ ID NO: 199 |
| | | QSVLFSSNNKNY | WAS | QQYYSTPPT |
| | CD98hc 15 | SEQ ID NO: 207 | SEQ ID NO: 208 | SEQ ID NO: 209 |
| | | QSVLFSSNNKNY | WAS | QQYYSTPPT |

### Tripod Construct Design

The antibodies generated against CD98 were screened for binding competition with CD98 and non-competitive mAbs formatted as scFv or nanobodies in a tripod mAb (also named TTP mAb) architecture and characterized. The tripod is used to deliver a substance of interest (e.g., a monoclonal antibody) to the brain. More specifically, a tripod construct (FIG. 1) containing a fusion of an antigen binding fragment of an antibody against CD98hc and a monoclonal antibody of interest (mAb) was developed to help the mAb penetrate the BBB and result in substantially higher brain concentrations of the mAb than the mAb alone.

For example, a tripod mAb consists of the therapeutic mAb with a CD98hc binding scFv or nanobody appended to the C-terminus of one antibody heavy chain using a short, flexible linker. Tripod mAbs were analysed for characteristics that have been previously described to enhance transcytosis (reviewed in (Goulatis and Shusta 2017): valency, binding affinity, pH dependent binding, and rapid internalization in brain endothelial cells.

Heavy and light chain variable sequences of an antibody against CD98hc were fused in a single genetic construct as the single-chain variable fragment (scFv) using the following format:
Hc_GTEGKSSGSGSESKST(SEQ ID NO: 189)_Lc

The scFv or VHH against CD98hc were then fused to the C-terminus of one heavy chain (Hc) of an antibody of interest using either GGSGGS (SEQ ID NO: 190) or GGAGGA (SEQ ID NO: 191) linker. The Zymeworks heterodimerization mutations in CH3 were utilized in the antibody He (Hc A: T350V_L351Y_F405A_Y407V; He B: T350V_T366L_K392L_T394W) to generate the tripod construct (FIG. 1), which is also referred to as a tripod mAb. A tripod mAb contains two light chains with the identical amino acid sequence and two heavy chains with different amino acid sequences. Only one of the two heavy chains is fused to a scFv or VHH of a CD98 antibody of the invention and the two heavy chains also differ in their constant regions to facilitate heterodimerization between the two heavy chains. Thus, each tripod mAb according to an embodiment of the application is associated with three amino acid sequences: the amino acid sequence of the first heavy chain fused to the antigen binding fragment of a CD98 antibody, the amino acid sequence of the light chain, and the amino acid sequence of the second heavy chain not fused to the antigen binding fragment of a CD98 antibody.

### Tripod Expression and Purification

Tripod mAbs were expressed in CHO-Expi cells and purified using Protein A affinity chromatography followed by Size Exclusion chromatography or Jon-exchange chromatography.

Examples of the tripod mAbs made are provided in Table 1a:

TABLE 1a

| | | scFv | VL | VH |
|---|---|---|---|---|
| | mAb component name | component | SEQ ID | SEQ ID |
| Tripod name | (target) | name | NO | NOs |
| BBBB404 | B21M IgG1 AAS | C98hc1 | 14 | 13/15 |
| BBBB1058 | Anti-Tau IgG1 AAS YTE | C98hc1 | 17 | 16/18 |
| BBB1056 | Anti-Tau IgG1 | C98hc1 | 20 | 19/21 |
| BBBB1097 | Anti-BAM IgG1 AAS YTE | C98hc1 | 23 | 22/24 |
| BBBB1226 | B21M IgG4 | C98hc1 | 26 | 25/27 |
| BBBB448 | B21M IgG1 AAS | C98hc2 | 33 | 32/34 |
| BBBB1061 | Anti-Tau IgG1 AAS YTE | C98hc2 | 36 | 35/37 |
| BBBB1059 | Anti-Tau IgG1 | C98hc2 | 39 | 38/40 |
| BBBB1100 | Anti-BAM IgG1 AAS YTE | C98hc2 | 42 | 41/43 |
| BBBB1228 | B21M IgG4 | C98hc2 | 45 | 44/46 |
| BBBB449 | B21M IgG1 AAS | C98hc3 | 52 | 51/53 |
| BBBB1064 | Anti-Tau IgG1 AAS YTE | C98hc3 | 55 | 54/56 |
| BBBB1062 | Anti-Tau IgG1 | C98hc3 | 58 | 57/59 |
| BBBB1103 | Anti-BAM IgG1 AAS YTE | C98hc3 | 61 | 60/62 |
| BBBB1229 | B21M IgG4 | C98hc3 | 64 | 63/65 |
| BBBB578 | B21M IgG1 AAS | C98hc4 | 74 | 73/75 |
| BBBB1067 | Anti-Tau IgG1 AAS YTE | C98hc4 | 77 | 76/78 |
| BBBB1065 | Anti-Tau IgG1 | C98hc4 | 80 | 79/81 |
| BBBB1106 | Anti-BAM IgG1 AAS YTE | C98hc4 | 83 | 82/84 |
| BBBB1230 | B21M IgG4 | C98hc4 | 86 | 85/87 |
| BBBB584 | B21M IgG1 AAS | C98hc5 | 96 | 95/97 |
| BBBB1070 | Anti-Tau IgG1 AAS YTE | C98hc5 | 99 | 98/100 |
| BBBB1068 | Anti-Tau IgG1 | C98hc5 | 102 | 101/103 |
| BBBB1109 | Anti-BAM IgG1 AAS YTE | C98hc5 | 105 | 104/106 |
| BBBB1231 | B21M IgG4 | C98hc5 | 108 | 107/109 |
| BBBB580 | B21M IgG1 AAS | C98hc6 | 120 | 119/121 |
| BBBB586 | B21M IgG1 AAS | C98hc7 | 130 | 129/131 |
| BBBB593 | B21M IgG1 AAS | C98hc8 | 140 | 139/141 |
| BBBB445 | B21M IgG1 AAS | C98hc9 | 147 | 146/148 |
| BBBB1227 | B21M IgG4 | C98hc9 | 220 | 219/221 |
| BBBB589 | B21M IgG1 AAS | C98hc10 | 157 | 156/158 |
| BBBB594 | B21M IgG1 AAS | C98hc11 | 167 | 166/168 |
| BBBB602 | B21M IgG1 AAS | C98hc12 | 177 | 176/178 |
| BBBB610 | B21M IgG1 AAS | C98hc13 | 187 | 186/188 |

### Cell Binding and CD98hc Specificity

Human brain endothelial cells (hCMECD3, 50,00 cells) were incubated with 10 ug/mL purified tripod mAb and allowed to incubate overnight at 4° C. in the presence of 10× molar concentration of huCD98hc (SEQ ID NO: 1). Cells were fixed and washed the following morning, incubated with secondary antibody (Jackson Immunosciences Cat#109-546-170), washed again and then analyzed by FACS. Positive binders were defined as having binding signal greater than 3-fold over isotype control (Table 2).

TABLE 2
hCMECD3 cell binding for tripod mAbs.

| mAb | SEQ ID NOs: | Rep 1 | Rep 2 | Ave Signal |
|---|---|---|---|---|
| BBBB404 | 13, 14, 15 | 11944 | 10148 | 11046 |
| BBBB448 | 32, 33, 34 | 12851 | 7458 | 10155 |
| BBBB449 | 51, 52, 53 | 7359 | 5521 | 6440 |
| BBBB578 | 73, 74, 75 | 23248 | 23583 | 23416 |
| BBBB584 | 95, 96, 97 | 3771 | 3079 | 3425 |
| BBBB580 | 119, 120, 121 | 13118 | 11030 | 12047 |
| Isotype control | 110, 111 | 371 | 123 | 247 |

### CD98hc Functional Assessment

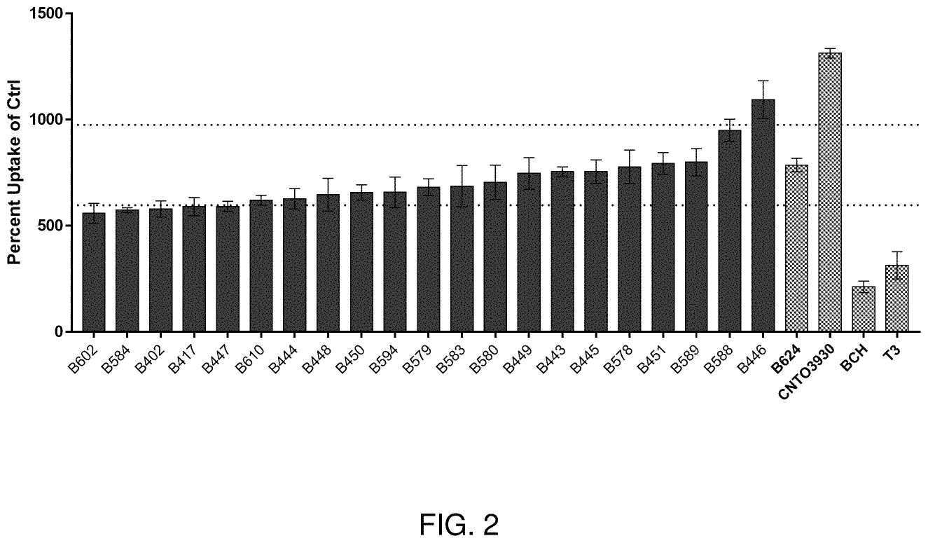

hCMECD3 cells were seeded into white, opaque 384-well plates and incubated overnight at 37° C. On the following day, the cells were washed and pulsed with sodium-free Ringer's Solution for 30 minutes at 37° C. The cells were washed once more with sodium-free Ringer's Solution and anti-CD98hc antibodies (140 nM), or LAT inhibitors BCH (50 mM) and T3 (200 μM), added to the cells. L-[3,4,5-3H(N)]-Leucine (0.4 μCi) was then added and after a 5-minute incubation, the cells were washed twice with ice-cold sodium-free Ringer's Solution and the cells lysed with 0.2% SDS in 0.2 N NaOH solution. The lysates were incubated with Microscint-PS solution for 1 hour at room temperature and scintillation counts obtained on a Perkin-Elmer TopCount NXT instrument. Criteria for antagonist activity was inhibition of ³H-leucine relative to the Zymeworks Tripod isotype control [Mean−(3×SD)] (FIG. 2). Based on the criteria, none of the test antibodies showed inhibition of ³H-leucine uptake, while both BCH and T3 LAT inhibitors showed significant inhibition of ³H-leucine uptake. CNTO3930 (hIgG1 isotype control) was included as additional negative control and showed no activity.

### Internalization

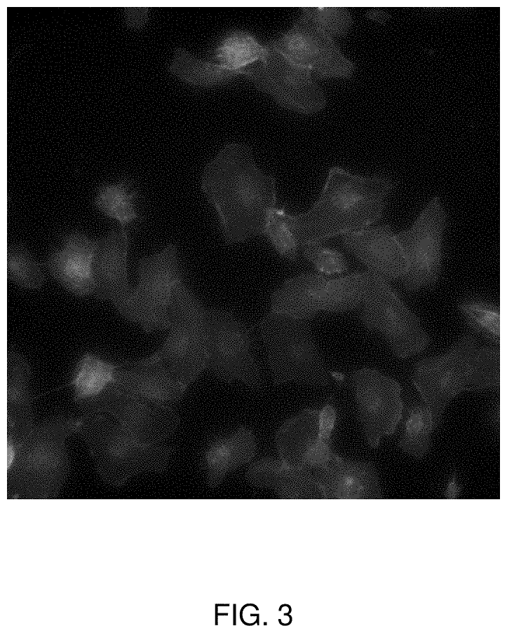

Human brain endothelial cells (hCMECD3) were plated at 10,000 cells/well in Collagen-coated 384-well Cell Carrier Ultra plates (Perkin Elmer) and allowed to attach for 16 hours at 37° C. in a humidified incubator. The cells (50,000 cells) were then incubated with 200 ug/mL purified tripod mAb and allowed to incubate at 37° C. for one hour. The cells were fixed, washed and incubated with a fluorescently labeled secondary antibody for one hour. The cells were then washed again and incubated with fluorescently labeled actin stain, Phalloidin, and nuclear stain, Hoeschst 33342. Cells were washed again and imaged using the ImageXpress Micro (Molecular Devices) with a 40× objective. Internalizing mAbs were identified on the basis of colocalization with Phalloidin using MetaXpress 6.0 (example in FIG. 3). All mAbs were positive for internalization.

Affinity Analysis & pH-Dependent Binding, Species Cross-Reactivity

To gain additional accuracy for affinity measurements, tripod mAb affinity for huCD98hc was determined using surface plasmon resonance (SPR) on a BioRad ProteOn XPR36 system. An Fc capture surface was generated by coupling anti-IgG Fc mAb (Jackson ImmunoResearch) to a GLC chip (BioRad) using the amine-coupling chemistry (BioRad).

Tripod mAbs were captured using a concentration of 0.3 ug/mL, flowed for 30 seconds at 60 uL/min for a target density of ˜200 RU. huCD98hc was then flowed over the immobilized tripod mAbs at concentrations from 6.25-400 nM, 800 nM, or 100-1600 nM (in a 4-fold serial dilution) for 4 min (at 50 μL/min) association followed by dissociation for 30 minutes at 50 uL/min. The chip surface was regenerated with two 18 second pulses of 100 mM H3PO4 (Sigma) at 100 μL/min. The collected data were processed using ProteOn Manager software V3.1.0.6 (BioRad). First, the data was corrected for background using inter-spots. Then, double reference subtraction of the data was performed by using the buffer injection for analyte injections. The kinetic analysis of the data was performed using a Langmuir 1:1 binding model. The result for each mAb was reported in the format of Ka (On-rate), Kd (Off-rate) and KD (equilibrium dissociation constant) (Table 3 and 3a).

TABLE 3
Binding affinity of tripod mAbs for huCD98hc

| Sample ID | SEQ ID NOs: | ka (1/Ms) | kd (1/s) | KD (nM) |
|---|---|---|---|---|
| BBBB404 | 13, 14, 15 | (4.23 ± 1.32)E−04 | (19.9 ± 4.01)E−03 | 5.03 ± 1.76 |
| BBBB445 | 146, 147, 148 | 7.10E+05 | 4.48E−03 | 6.31 |
| BBBB448 | 32, 33, 34 | 6.50E+05 | 1.49E−03 | 2.29 |
| BBBB449 | 51, 52, 53 | 8.33E+05 | 2.79E−03 | 3.35 |
| BBBB578 | 73, 74, 75 | 3.90E+05 | 1.54E−03 | 3.95 |
| BBBB584 | 95, 96, 97 | 5.00E+04 | 5.65E−03 | 1.13 |
| BBBB580 | 119, 120, 121 | 1.88E+05 | 9.65E−03 | 51.4 |
| BBBB586 | 129, 130, 131 | (3.00 ± 0.23)E+05 | (2.16 ± 0.05)E−03 | 7.23 |
| BBBB589 | 156, 157, 158 | 6.36E+05 | 2.63E−02 | 41.3 |
| BBBB593 | 139, 140, 141 | (1.05 ± 0.04)E+05 | (3.44 ± 0.14)E−03 | 32.80 |
| BBBB594 | 166, 167, 168 | 3.48E+05 | 1.39E−03 | 4.00 |
| BBBB602 | 176, 177, 178 | 3.99E+04 | 8.71E−03 | 218 |
| BBBB610 | 186, 187, 188 | 6.10E+05 | 8.89E−04 | 1.46 |
| BBBB1201 | 200, 201, 202 | 3.57E+05 | 6.00E−03 | 16.85 |
| BBBB1202 | 210, 211, 212 | 1.67E+05 | 8.82E−03 | 52.90 |

TABLE 3a
SPR binding for IgG4 mAbs

| Name | Description | ka (1/Ms) | kd (1/s) | KD (M) |
|---|---|---|---|---|
| BBBB1227 | B21M IgG4 ZW BBBB445 | (4.40 ± 0.17)E+05 | (2.42 ± 0.64)E−03 | (5.49 ± 1.23)E−09 |
| BBBB1228 | B21M IgG4 ZW BBBB448 | (4.26 ± 1.13)E+05 | (1.77 ± 0.71)E−03 | (4.21 ± 2.34)E−09 |
| BBBB1229 | B21M IgG4 ZW BBBB449 | (4.82 ± 1.34)E+05 | (3.36 ± 2.38)E−03 | (6.88 ± 3.14)E−09 |

Results are Average ± 95% Cl

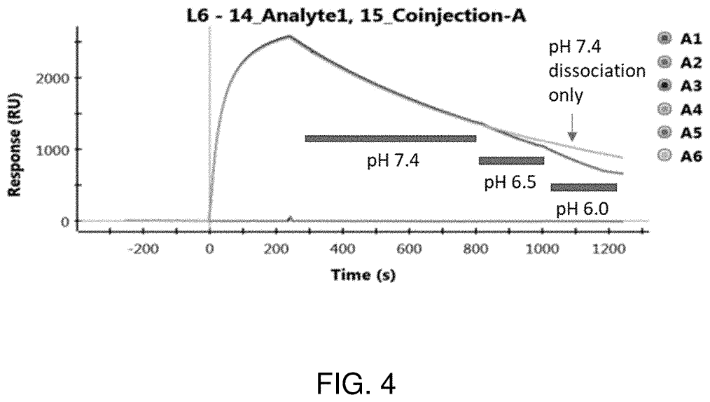

pH dependent binding was assessed using the SPR (proteon) method described above, except biotinylated CD98hc was immobilized on a streptavidin chip, and during the dissociation, the buffer pH was stepped down from 7.4 to 6.5 to 6.0. The individual sensorgrams were evaluated and scored for pH dependent binding if the off-rate was faster as the pH decreased (example in FIG. 4).

Species cross-reactivity was assessed using the same method as for determining binding affinity, except the CD98hcs used were cyno (SEQ ID NO: 2), marmoset (SEQ ID NO: 3), mouse (SEQ ID NO: 4) and rat (SEQ ID NO: 5). No rat or mouse cross-reactive mAbs were identified. Cyno and marmoset cross-reactive tripod mAbs were identified (Table 4).

TABLE 4
Species cross-reactivity for selected tripod mAbs

| | | | Marmoset KD | |
|---|---|---|---|---|
| mAb | SEQ ID NOs: | Cyno KD (M) | (M) | Human KD (M) |
| BBBB404 | 13, 14, 15 | 4.54E−08 | No binding | 5.03E−09 |
| BBBB445 | 146, 147, 148 | 6.31E−09 | 1.38E−08 | 2.28E−09 |
| BBBB448 | 32, 33, 34 | 5.32E−09 | 9.45E−09 | 2.29E−09 |
| BBBB449 | 51, 52, 53 | 7.56E−09 | 1.97E−08 | 3.35E−09 |
| BBBB578 | 73, 74, 75 | 6.22E−08 | No binding | 3.95E−09 |
| BBBB584 | 95, 96, 97 | 1.85E−07 | 4.70E−07 | 1.13E−07 |
| BBBB586 | 119, 120, 121 | (2.02 ± 0.15)E−08 | Not measured | (7.23 ± 0.56)E−09 |
| BBBB589 | 129, 130, 131 | 4.13E−08 | No binding | 9.17E−09 |
| BBBB593 | 156, 157, 158 | (3.35 ± 0.08)E−07 | Not measured | (3.28 ± 0.18)E−08 |
| BBBB594 | 139, 140, 141 | 4.00E−09 | No binding | 2.17E−09 |
| BBBB602 | 166, 167, 168 | 2.18E−07 | Weak binding | 1.28E−07 |
| BBBB610 | 176, 177, 178 | 1.46E−09 | 8.39E−08 | 2.18E−09 |

Selection of mAbs for Cyno Studies and Assessment of Brain Shuttles Fused to Anti-Tau mAb

Critical to confirming the ability of the CD98hc targeting tripod mAbs to enhance therapeutic antibody brain exposure in humans is demonstrating enhanced brain delivery in non-human primates. BBBB578, BBBB448 and BBBB584 were selected for the cyno PK study. The anti-CD98hc scFv was fused to the anti-Tau mAb, PT1B844 (SEQ ID NOs: 110, 111) to generate BBBB1067/BBBB1065 (SEQ ID NOs: 76, 77, 78/SEQ ID NOs: 79, 80, 81), BBBB1061 (SEQ ID NOs: 35, 36, 37) and BBBB1070 (SEQ ID NOs: 98, 99, 100). The affinity for huCD98hc was measured (Table 5) and was similar to binding when fused to an anti-B21M mAb.

TABLE 5
Binding affinity for huCD98hc of anti-CD98hc brain
shuttles fused to the anti-Tau mAb

| mAb Fusion | SEQ ID NOs: | ka (1/Ms) | kd (1/s) | KD (M) |
|---|---|---|---|---|
| BBBB1065 | 79, 80, 81 | (4.70 ± 0.21)E+05 | (1.39 ± 0.02)E−03 | (2.96 ± 0.17)E−09 |
| BBBB1067 | 76, 77, 78 | (4.39 ± 0.15)E+05 | (1.38 ± 0.03)E−03 | (3.15 ± 0.13)E−09 |
| BBBB1070 | 98, 99, 100 | (5.57 ± 0.94)E+04 | (6.48 ± 0.82)E−03 | (1.18 ± 0.21)E−07 |
| BBBB1061 | 35, 36, 37 | (9.35 ± 1.16)E+05 | (1.35 ± 0.09)E−03 | (1.46 ± 0.18)E−09 |

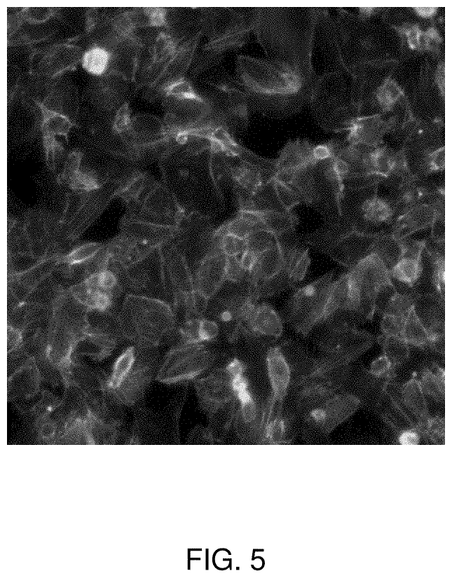

Similar to previous studies, internalization was also assessed and fusion of the brain shuttle to anti-Tau mAb did not impact its ability to internalize in human brain endothelial cells (FIG. 5).

Cyno Pharmacokinetics of Anti-Tau Brain Shuttle mAbs

Cynomolgus monkeys were administered with test articles by IV injection (slow bolus) with the indicated dose. At the scheduled timepoints, cynomolgus monkey brain was collected and rinsed with cold saline solution following upper body perfusion with saline for minimum of 5 minutes. Predefined brain locations were isolated, snap frozen in liquid nitrogen, and stored at −80° C. until tissue homogenization and capillary depletion processing.

BBBB1067, BBBB1061 and BBB1070 were dosed i.v. at 10 mg/kg along with the non-brain shuttle-enabled mAbs PT1B844 and PT1B916 in cynomolgus monkeys. Plasma was sampled at 4, 24 and 72 hours. Cynomolgus monkey brain was collected and rinsed with cold saline solution following upper body perfusion with saline for minimum of 5 minutes. Predefined brain locations were isolated, snap frozen in liquid nitrogen, and stored at −80° C. until tissue homogenization and capillary depletion processing.

For sample preparation of the capillary-depleted brain tissue lysates, individual tissue weights were obtained for the brain locations collected. The brain tissue samples were added to a calculated volume of modified dPBS buffer (2.5 μL buffer/1 mg tissue) containing protease inhibitor (Pierce; A32955) and transferred to Lysing Matrix D (MP Biomedicals™; 6913-100) tubes. Tissue samples were homogenized at 2.9 m/s for 15 seconds using a Bead Ruptor 24 Elite (Omni International). The total cell suspension was transferred into a new tube and mixed with an equal volume of a 26% dextran buffer (13% final dextran concentration). Mixed tissue homogenate was centrifuged at 2,000 g for 10 minutes at 4° C. Carefully, the upper layer (capillary-depleted fraction) was separated from the remaining sample and transferred to a new tube containing 10×RIPA lysis buffer (Millipore™; 20-188). Capillary-depleted samples plus lysis buffer were vortexed well, centrifuged at 14,000 rpm for 30 minutes at 4° C., and supernatant transferred to a new tube. Brain tissue sample lysates were either stored frozen at −70° C. or measured for protein concentrations using BCA protein assay kit (Pierce™; 23227). Final brain tissue sample lysates were normalized to 7 mg/mL total protein concentration prior to immunoassay determination of BBB-enabled mAbs.

The concentration of BBB-enabled mAbs in cynomolgus monkey brain tissue for PK assessment was determined using MesoScale Discovery (MSD®; Gaithersburg, Md.) ECLIA technology developed in a typical sandwich immunoassay format. The assay was performed on MSD Gold™ Small Spot Streptavidin 96-well plates (Cat: L45SA). The streptavidin-coated plates were blocked with 1% bovine serum albumin (BSA) in 1× phosphate buffered saline (PBS) for 30 minutes at room temperature. The standard curve was prepared fresh in 50% naïve cyno brain tissue lysates by serial dilution. Frozen quality controls (QCs) prepared in naïve cyno brain tissue lysates at 2× of the working assay concentration were diluted and tested with each assay. Master mix containing the capture (biotinylated anti-human Fc mAb, 1 μg/mL) and detection (ruthenium-labeled anti-human Fc mAb, 0.5 μg/mL) reagents was combined with diluted standards, QCs, and samples at a 1:1 volume ratio in the assay plate. The mixture was incubated for 1 hour with shaking at room temperature. Assay plates were washed, and MSD T read buffer (1×) was added to all wells. Raw data values were read on an MSD SECTOR® S600 imager. The standard curve range for the assay was tested at 1-512 ng/mL with a minimum required sample dilution (MRD) of 1:2, yielding a limit of sensitivity of 2 ng/mL in brain tissue lysates. The MSD output files with the raw ECL counts were imported into Watson LIMS (Thermo Scientific) and then regressed with a 5-parameter logistic fit with 1/F2 weighting.

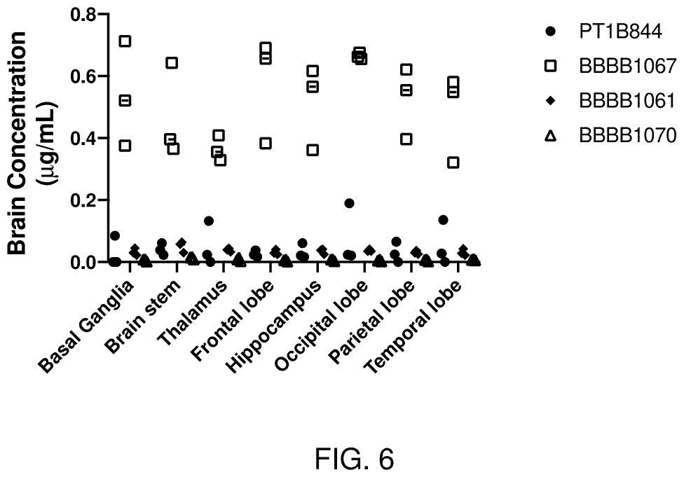

Brain concentration was determined for the four mAbs across a variety of areas (FIG. 6). All brain shuttle containing mAbs had increased brain exposure over the non-brain shuttle containing mAbs in every region of the brain. When data were converted to % injected dose/kg brain and averaged across regions, a 16× improvement in brain concentration was realized for the brain shuttle mAbs.

Due to the widespread tissue expression of CD98hc, a tripod mAb capable of binding to FcgRs could induce ADCC/ADCP on a variety of tissues. To avoid this possibility an effector silent Fc was utilized by introducing the “AAS” mutations into the tripod mAb. While avoiding ADCC/ADCP is an important characteristic for the safety of the therapeutic mAb, one potential therapeutic mechanism of action relies on Fc-dependent microglia phagocytosis of Tau aggregates. By inhibiting the ability of the brain shuttle mAb to bind to FcγR, the mAb would not be able to bind FcγR on microglia cells to promote phagocytosis of Tau aggregates. However, a novel mechanism of microglia uptake is exploited by our brain shuttle through CD98hc-mediated, non-Fc dependent internalization.

To assess the potential of the AAS IgG1 tripod mAb to promote uptake of Tau aggregates in microglia cell, human microglia derived from Induced Pluripotent Stem Cells (iPSC) were plated onto 384 well Perkin Elmer Cell Carrier Ultra plates at a dilution of 7000 cells per plate and maintained in advanced DMEM/F12 media with Glutamax+, Penicillin/Streptomycin, IL34 (100 ng/ml) and GMCSF (10 ng/ml). On the day of the assay, biotinylated phospho-tau oligomers [sequence: SCBiot-(dPEG4)GTPGSRSR(pT)PSLP(pT) PPTREPLL-amide (SEQ ID NO: 192)] were allowed to complex with streptavidin Alexa Flour 488 (AF488) at 15-fold molar excess. Labelled phospho-tau oligomers were then allowed to bind test mAbs at approximately 2× molar excess at room temperature for 30 minutes. The mAb:tau oligomer complex was then delivered to microglia at 20 μl/well. At 2, 4, and 8 hours post incubation, cells were washed twice with phosphate buffered saline (PBS) and fixed in the presence of 4% paraformaldehyde for 15 minutes at room temperature. Following fixation, cells were once again washed twice in PBS and incubated overnight with LAMP1 primary antibody, a marker for lysosomes, at a concentration of 4 μg/ml in permeabilization buffer (0.1% saponin+1% Fish skin gelatin) at 4° C. Post incubation, cells were washed twice with PBS and stained with 1 μg/ml secondary antibody conjugated to Alexa flour 647 in permeabilization buffer for 1 hour at 4° C. Post incubation, cells were washed twice with PBS, counter stained with Hoechst DNA stain at 1 μg/ml for 10 minutes at room temperature in PBS. The cells were then washed one final time in PBS, resuspended in 20 μl of PBS per well, and imaged on the Opera Phenix confocal high content microscope. Acquired images were analyzed using Harmony and ImageJ analysis software. Approximately 500 cells per condition were scored for the presence of Tau oligomers within phagolysosomal structures, labelled with LAMP1 antibody.

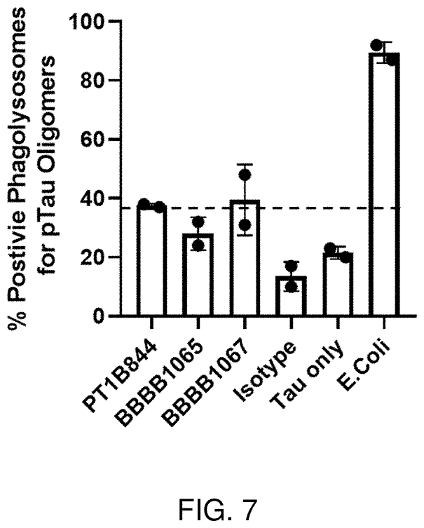

The brain shuttle mAb BBBB1067 promoted equivalent uptake into phagosomes than the non-brain shuttle mAb, PT1B844 (FIG. 7), with BBBB1065 unexpectedly worse. These data demonstrate that eliminating binding to FcγR should not impact therapeutic efficacy of the anti-Tau mAbs, in fact CD98hc-mediated internalization and trafficking to the phagolysosome appears more efficient in microglia than did traditional FcγR mediated phagocytosis.

### Discussion

To achieve an optimized brain delivery platform based on receptor mediated transcytosis, mAbs were generated that bind specifically to the human CD98hc with a range of affinities in a pH-dependent manner. The relationship between binding affinity and transcytosis efficiency has been covered extensively in numerous publications with a focus on the equilibrium dissociation constant, K_{D}. While K_{D} is an important measure, it has been surprisingly demonstrated in the invention the criticality of the binding kinetics, kₐ and k_{d}, for transcytosis. Inventors discovered that both on- and off-rates need to be optimized for efficient transcytosis and pharmacodynamic activity of the therapeutic mAb delivered. Based on the results, optimal transcytosis occurs when, for example, the kₐ≥10⁵ M⁻¹ s⁻¹ and neutral k_{d}=8×10⁻³ sec⁻¹. While not wishing to be bound by theories, it is hypothesized the interplay between on-rate and off-rate is critical to ensuring efficient transcellular transport through the diverse intracellular vesicles responsible for protein trafficking in polarized cells.

It has been demonstrated that dosing tripod mAbs in cynomolgus monkeys results in 16× enhancement in brain concentration over a control mAb. Increasing acidic FcRn binding resulted in reduced peripheral clearance and enhanced brain concentration. Under normal physiological conditions, FcRn-mediated efflux of antibodies from the brain is likely crucial in maintaining brain homeostasis by avoiding unwanted inflammation and immune responses in the brain (Schlachetzki, Zhu et al. 2002, Roopenian and Akilesh 2007). While the preponderance of evidence suggests a strong role for FcRn-mediated efflux of antibodies, there does remain some debate about this clearance mechanism (Garg and Balthasar 2009, Abuqayyas and Balthasar 2013). Inventors discovered that increasing the binding affinity for FcRn has a positive impact on both peripheral and brain concentration, suggesting that any enhanced efflux is insignificant in this system.

The clear disadvantage to Fc mutagenesis is the elimination of effector function from the therapeutic mAb. For many therapeutic targets in the brain, like beta-amyloid and Tau, ADP is believed critical to efficacy. Inventors demonstrated that this endogenous diversion of multivalent cargo can be used as an alternative, non-classical, non-FcγR mechanism of ADP. Tau internalized through non-classical and classical ADP are trafficked similarly in microglia, with Tau aggregates trafficking through the endolysosomal system to the lysosome for degradation. The described non-classical ADP can be exploited for a variety of therapeutic applications where ADP is necessary for efficacy but classical ADP harmful for safety.

The data indicate that non-classical ADP is more efficient than classical ADP Observations for macrophage exhaustion have been made in vitro and in patients (Baig, 2014), indicating that this exhaustion phenotype may impact therapeutic efficacy of mAbs with effector function. The non-classical ADP offers an efficacy advantage, avoiding this exhaustion phenotype by mediating ADP without activating microglia by binding FcγRs.

Another advantage of non-classical ADP is that by avoiding microglial activation ADP occurs without stimulating the production pro-inflammatory cytokines. There remains debate on the safety of using effector-function competent mAbs in treating diseases in the brain, particularly around increasing neuroinflammation in patients who already suffer from chronic neuroinflammation (reviewed in Heneka, et al. Lancet Neurol. 2015 April; 14(4): 388-405). In addition, there is increasing attention to the role that inflammation plays in the pathogenesis of neurodegenerative disease with the implications of increasing inflammation, as well as, the ability to engage or further activate potentially already exhausted microglia under debate. For example, the toxic impact of classic ADP on neurons was been demonstrated and hypothesized that effector function competent mAbs may pose safety risks (Lee, et al. 2016). In conclusion, a robust brain delivery platform has been characterized for pharmacokinetics, pharmacodynamics and safety establishing the robust preclinical characterization needed to advance to clinical trials.

The optimized brain shuttles were fused to PT1B844, a Tau binding mAb. Brain shuttles fused to PT1B844 demonstrated a 16-fold improvement in brain concentration when dosed i.v. in cynomolgus monkey. The enhancement in brain concentration exceeded the best brain shuttles reported in literature. In addition to superior brain PK, the brain shuttles are engineered to reduce Fc-mediated effector function and do not induce rapid reticulocyte depletion in cynomolgus as has been reported by competitors. Importantly, the loss of Fc-function doesn't impact the effectiveness of the therapeutic Tau mAb, as the brain shuttle is more efficient at mediating microglial uptake of Tau than PT1B844 alone.

### Example 2

Selected anti-CD98 brain shuttles will be fused to a prototypical anti-BACE (β-secretase) mAb and binding affinity reassessed using same method as described above. The affinity of the anti-CD98 brain shuttles is expected to be similar when fused to B21M mAb (anti-human respiratory syncytial virus) and anti-BACE antagonist mAb. Internalization will be assessed for selected molecules and expected to be found unchanged from internalization observed when the anti-CD98 brain shuttle was fused to B21M mAb.

Since none of the anti-CD98 brain shuttles bound to mouse or rat CD98, in vivo rodent studies will be conducted in huCD98hc knock-in (KI) mice using an anti-BACE antagonist mAb. The anti-BACE antagonist mAb was selected as a model PD system for measuring inhibition of BACE1 (through the concentration of its product peptide, AP1-40), a reflection of the amount of mAb that is trafficked to the brain.

The first in vivo study will assess the PK/PD relationship in the retina of huCD98hc mice. The knock-in (KI) mice will be dosed at 10 mg/kg i.v. with the anti-BACE antagonist mAb and a control mAb. Eyes and plasma will be harvested at 4- and 24-hours following dosing. At the scheduled timepoints, mice will be anesthetized by inhalation of isoflurane. Mouse eyes from KI mice will be collected following whole-body perfusion with 5 mL of 0.9% saline solution. The collected eye sample (minus the optic nerve) will be snap frozen in liquid nitrogen, and stored at −70° C. until tissue homogenization or prepared for immunohistochemistry.

BACE activity measurements will be made by homogenizing mouse eyes in lysing matrix D tube (8 μl of 0.4% DEA/50 mM NaCl per mg of brain weight, Fast Prep-24 at 6/shakes/sec for 20 sec). Tubes will then be centrifuged at 4° C. for 5 min in an Eppendorf Centrifuge set to a maximum speed. Homogenate (supernatant) will then transferred to precooled tubes which were then centrifuged for 70 minutes at 13,000 rpm at 4° C. Supernatant will then be transferred to a tube containing 10% of 0.5 M Tris/HCL and frozen at −80° C. until assayed. Aβ 1-40 peptide standards and thawed processed eye homogenate are then pre-complexed at 1:1 with ruthenium (Meso Scale Discovery (MSD), R91AN-1) labeled anti-Aβ antibody. 50 ul of complex will be added to blocked plate containing capture antibody to Aβ 1-40. After overnight incubation at 2-8° C. with no shaking, plates will be washed and 2× read buffer (MSD, R92TC-1) added. Plate will be read using Meso Sector S 600 (MSD, ICOAA).

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

### CITATIONS

Abuqayyas, L. and J. P. Balthasar (2013). “Investigation of the role of FcgammaR and FcRn in mAb distribution to the brain.” Mol Pharm 10(5): 1505-1513.
Church, A. K., K. R. VanDerMeid, N. A. Baig, A. M. Baran, T. E. Witzig, G. S. Nowakowski and C. S. Zent (2016). “Anti-CD20 monoclonal antibody-dependent phagocytosis of chronic lymphocytic leukaemia cells by autologous macrophages.” Clin Exp Immunol 183(1): 90-101.
Cooper, P. R., G. J. Ciambrone, C. M. Kliwinski, E. Maze, L. Johnson, Q. Li, Y. Feng and P. J. Hornby (2013). “Efflux of monoclonal antibodies from rat brain by neonatal Fc receptor, FcRn.” Brain Res 1534: 13-21.
Dall'Acqua, W. F., P. A. Kiener and H. Wu (2006). “Properties of human IgG1s engineered for enhanced binding to the neonatal Fc receptor (FcRn).” J Biol Chem 281(33): 23514-23524.
Garg, A. and J. P. Balthasar (2009). “Investigation of the influence of FcRn on the distribution of IgG to the brain.” AAPS J 11(3): 553-557.
Goulatis, L. I. and E. V. Shusta (2017). “Protein engineering approaches for regulating blood-brain barrier transcytosis.” Curr Opin Struct Biol 45: 109-115.
Johnsen, K. B., A. Burkhart, F. Melander, P. J. Kempen, J. B. Vejlebo, P. Siupka, M. S. Nielsen, T. L. Andresen and T. Moos (2017). “Targeting transferrin receptors at the blood-brain barrier improves the uptake of immunoliposomes and subsequent cargo transport into the brain parenchyma.” Sci Rep 7(1): 10396.
Robbie, G. J., R. Criste, W. F. Dall'acqua, K. Jensen, N. K. Patel, G. A. Losonsky and M. P.
Griffin (2013). “A novel investigational Fc-modified humanized monoclonal antibody, motavizumab-YTE, has an extended half-life in healthy adults.” Antimicrob Agents Chemother 57(12): 6147-6153.
Roopenian, D. C. and S. Akilesh (2007). “FcRn: the neonatal Fc receptor comes of age.” Nat Rev Immunol 7(9): 715-725.
Schlachetzki, F., C. Zhu and W. M. Pardridge (2002). “Expression of the neonatal Fc receptor (FcRn) at the blood-brain barrier.” J Neurochem 81(1): 203-206.
Vandermeeren, M., M. Borgers, K. Van Kolen, C. Theunis, B. Vasconcelos, A. Bottelbergs, C. Wintmolders, G. Daneels, R. Willems, K. Dockx, L. Delbroek, A. Marreiro, L. Ver Donck, C. Sousa, R. Nanjunda, E. Lacy, T. Van De Casteele, D. Van Dam, P. P. De Deyn, J. A. Kemp, T. J. Malia and M. H. Mercken (2018). “Anti-Tau Monoclonal Antibodies Derived from Soluble and Filamentous Tau Show Diverse Functional Properties in vitro and in vivo.” J Alzheimers Dis 65(1): 265-281.
Yu, Y. J., J. K. Atwal, Y. Zhang, R. K. Tong, K. R. Wildsmith, C. Tan, N. Bien-Ly, M. Hersom, J. A. Maloney, W. J. Meilandt, D. Bumbaca, K. Gadkar, K. Hoyte, W. Luk, Y. Lu, J. A. Ernst, K. Scearce-Levie, J. A. Couch, M. S. Dennis and R. J. Watts (2014). “Therapeutic bispecific antibodies cross the blood-brain barrier in nonhuman primates.” Sci Transl Med 6(261): 261ra154.
Yu, Y. J., Y. Zhang, M. Kenrick, K. Hoyte, W. Luk, Y. Lu, J. Atwal, J. M. Elliott, S. Prabhu, R. J. Watts and M. S. Dennis (2011). “Boosting brain uptake of a therapeutic antibody by reducing its affinity for a transcytosis target.” Sci Transl Med 3(84): 84ra44.
Zent, C. S. and M. R. Elliott (2017). “Maxed out macs: physiologic cell clearance as a function of macrophage phagocytic capacity.” FEBS J 284(7): 1021-1039.

We claim:

1. An anti-CD98 antibody or antigen-binding fragment thereof for delivering an agent to the brain of a subject in need thereof, wherein the anti-CD98 antibody or antigen-binding fragment thereof binds to a CD98, preferably human CD98hc, with a dissociation constant K_{D} of at least 1 nM, preferably 1-500 nM, at neutral pH and an off-rate constant k_{d} of at least 10⁻⁴ sec⁻¹, preferably 10⁻⁴ to 10⁻¹ sec⁻¹, at an acidic pH, preferably pH 5.

2. The anti-CD98 antibody or antigen-binding fragment thereof of claim 1, having an off-rate constant k_{d} of 2×10⁻² to 2×10⁻⁴ sec⁻¹, preferably 8×10⁻³ sec⁻¹ at the neutral pH.

3. The anti-CD98 antibody or antigen-binding fragment thereof of claim 1 or 2, comprising:
(1) a heavy chain variable region comprising heavy chain complementarity determining regions (HCDRs) HCDR1, HCDR2 and HCDR3, and a light chain variable region comprising light chain complementarity determining regions (LCDRs) LCDR1, LCDR2 and LCDR3, wherein the HCDR1, HCDR2, HCDR3, LCDR1, LCDR2 and LCDR3 have the amino acid sequences of:
(i) SEQ ID NOs: 7, 8, 9, 10, 11 and 12, respectively;
(ii) SEQ ID NOs: 67, 68, 69, 70, 71 and 72, respectively;
(iii) SEQ ID NOs: 89, 90, 91, 92, 93 and 94, respectively;
(iv) SEQ ID NOs: 113, 114, 115, 116, 117 and 118, respectively;
(v) SEQ ID NOs: 123, 124, 125, 126, 127 and 128, respectively;
(vi) SEQ ID NOs: 133, 134, 135, 136, 137 and 138, respectively;
(vii) SEQ ID NOs: 150, 151, 152, 153, 154 and 155, respectively;
(viii) SEQ ID NOs: 160, 161, 162, 163, 164 and 165, respectively;
(ix) SEQ ID NOs: 170, 171, 172, 173, 174 and 175, respectively;
(x) SEQ ID NOs: 180, 181, 182, 183, 184 and 185, respectively;
(xi) SEQ ID NOs: 194, 195, 196, 197, 198 and 199, respectively; or
(xii) SEQ ID NOs: 204, 205, 206, 207, 208 and 209, respectively; or
(2) a single variable domain on a heavy chain (VHH) comprising heavy chain complementarity determining regions (HCDRs) HCDR1, HCDR2 and HCDR3 having the amino acid sequences of:
(i) SEQ ID NOs: 29, 30 and 31, respectively;
(ii) SEQ ID NOs: 48, 49 and 50, respectively;
(iii) SEQ ID NOs: 143, 144 and 145, respectively.

4. The anti-CD98 antibody or antigen-binding fragment thereof of any one of claims 1-3, being a VHH fragment comprising an amino acid sequence having at least 80%, such as at least 85%, 90%, 95% or 100%, sequence identity to SEQ ID NO: 28, SEQ ID NO: 47, or SEQ ID NO: 142.

5. The anti-CD98 antibody or antigen-binding fragment thereof of any one of claims 1-3, being single-chain variable fragment (scFv) comprising the heavy chain variable region covalently linked to the light chain variable region via a linker, preferably, the linker has the amino acid sequence of SEQ ID NO: 189, more preferably, the scFv comprises an amino acid sequence having at least 80%, such as at least 85%, 90%, 95% or 100%, sequence identity to the amino acid sequences of SEQ ID NO: 6, SEQ ID NO: 66, SEQ ID NO: 88, SEQ ID NO: 112, SEQ ID NO: 122, SEQ ID NO: 132, SEQ ID NO: 149, SEQ ID NO: 159, SEQ ID NO: 169, SEQ ID NO: 179, SEQ ID NO: 193, or SEQ ID NO: 203.

6. A conjugate comprising the anti-CD98 antibody or antigen-binding fragment thereof of any one of claims 1-5 coupled to a therapeutic or diagnostic agent, preferably, the conjugate is a multi-specific antibody comprising a first antigen binding region which binds the CD98 and comprises the antigen-binding fragment of any one of claims 1 to 5, and a second antigen binding region which binds a brain target, such as a brain target selected from the group consisting of beta-secretase 1 (BACE1), amyloid beta (Abeta), epidermal growth factor receptor (EGFR), human epidermal growth factor receptor 2 (HER2), Tau, apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), and caspase 6.

7. A fusion construct comprising the anti-CD98 antibody or antigen-binding fragment thereof of any one of claims 1 to 5 covalently linked to a second antibody or an antigen binding fragment thereof that binds to a brain target, such as a brain target selected from the group consisting of beta-secretase 1 (BACE1), amyloid beta (Abeta), epidermal growth factor receptor (EGFR), human epidermal growth factor receptor 2 (HER2), Tau, apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), and caspase 6.

8. The fusion construct of claim 7, wherein the anti-CD98 antibody or antigen-binding fragment thereof is covalently linked to the carboxy terminus of only one of the two heavy chains of the second antibody or antigen binding fragment thereof via a linker, preferably wherein the linker has the amino acid sequence of SEQ ID NO: 190 or SEQ ID NO: 191.

9. The fusion construct of claim 8, wherein each of the two heavy chains of the second antibody or antigen binding fragment thereof comprises one or more heterodimeric mutations, such as a modified heterodimeric CH3 domain, or one or more knob and hole mutations, as compared to a wild-type CH3 domain polypeptide.

10. The fusion construct of claim 9, wherein the heterodimeric mutations comprise the modified heterodimeric CH3 domain of the first heavy chain comprises amino acid modifications at positions T350, L351, F405, and Y407, and the modified heterodimeric CH3 domain of the second heavy chain comprises amino acid modifications at positions T350, T366, K392 and T394, wherein the amino acid modification at position T350 is T350V, T350I, T350L or T350M; the amino acid modification at position L351 is L351Y; the amino acid modification at position F405 is F405A, F405V, F405T or F405S; the amino acid modification at position Y407 is Y407V, Y407A or Y407I; the amino acid modification at position T366 is T366L, T366I, T366V or T366M, the amino acid modification at position K392 is K392F, K392L or K392M, and the amino acid modification at position T394 is T394W, and wherein the numbering of amino acid residues is according to the EU index as set forth in Kabat.

11. The fusion construct of claim 10, wherein the modified heterodimeric CH3 domain of the first heavy chain comprises mutations T350V, L351Y, F405A and Y407V, and the modified heterodimeric CH3 domain of the second heavy chain comprises mutations T350V, T366L, K392L and T394W.

12. The fusion of any one of claims 7-11, wherein the second antibody or antigen binding fragment thereof comprises one or more mutations in the Fc domain that enhance binding of the fusion to the neonatal Fc receptor (RcRn), preferably the one or more mutations enhance the binding at an acidic pH, more preferably the Fc has the M252Y/S254T/T256E (YTE) mutations, wherein the numbering of amino acid residues is according to the EU index as set forth in Kabat.

13. The fusion of any one of claims 7-12, wherein the second antibody or antigen binding fragment thereof comprises one or more mutations in the Fc domain that reduce or eliminate the effector function, preferably the Fc has one or more amino acid modifications at positions L234, L235, D270, N297, E318, K320, K322, P331, and P329, such as one, two or three mutations of L234A, L235A and P331S, wherein the numbering of amino acid residues is according to the EU index as set forth in Kabat.

14. The fusion construct of any one of claims 7-13, wherein the second antibody or antigen binding fragment thereof binds to Tau and comprises HCDR1, HCDR2, HCDR3, LCDR1, LCDR2 and LCDR3 having the amino acid sequences of SEQ ID NOs: 213 to 218, respectively, preferably, the second antibody is a monoclonal antibody comprising a heavy chain having the amino acid sequence of SEQ ID NO: 110 and a light chain having the amino acid sequence of SEQ ID NO: 111.

15. The fusion construct of any one of claims 7-14, comprising:
(1) a first heavy chain having an amino acid sequence that is at least 80%, such as at least 85%, 90%, 95% or 100%, identical to an amino acid sequence selected from the group consisting of SEQ ID NOs: 13, 16, 19, 22, 25, 32, 35, 38, 41, 44, 51, 54, 57, 60, 63, 73, 76, 79, 82, 85, 95, 98, 101, 104, 107, 119, 129, 139, 146, 156, 166, 176, 186, 200, 210, and 219;
(2) two light chains each independently having an amino acid sequence that is at least 80%, such as at least 85%, 90%, 95% or 100%, identical to an amino acid sequence selected from the group consisting of SEQ ID NOs: 14, 17, 20, 23, 26, 33, 36, 42, 45, 52, 55, 58, 61, 64, 74, 77, 80, 83, 86, 96, 99, 102, 105, 108, 120, 130, 140, 147, 157, 167, 177, 187, 201, 211, and 220, respectively; and
(3) a second heavy chain having an amino acid sequence that is at least 80%, such as at least 85%, 90%, 95% or 100%, identical to an amino acid sequence selected from the group consisting of SEQ ID NOs: 15, 18, 21, 24, 27, 34, 37, 43, 46, 53, 56, 59, 62, 65, 75, 78, 81, 84, 87, 97, 100, 103, 106, 109, 121, 131, 141, 148, 158, 168, 178, 188, 202, 212, and 221, respectively.

16. An isolated nucleic acid encoding the antibody or antigen-binding fragment of any one of claims 1-5, the conjugate of claim 6 or the fusion construct of any one of claims 7-15.

17. A vector comprising the isolated nucleic acid of claim 16.

18. A host cell comprising the nucleic acid of claim 16 or the vector of claim 17.

19. A method of producing the antibody or antigen-binding fragment of any one of claims 1-5, the conjugate of claim 6, or the fusion construct of any one of claims 7-15, comprising culturing a cell comprising a nucleic acid encoding the antibody or antigen-binding fragment, the conjugate or the fusion construct under conditions to produce the antibody or antigen-binding fragment, the conjugate or the fusion construct, and recovering the antibody or antigen-binding fragment, the conjugate or the fusion construct from the cell or cell culture.

20. A pharmaceutical composition comprising the antibody or antigen-binding fragment of any one of claims 1-5, the conjugate of claim 6, or the fusion construct of any one of claims 7-15, and a pharmaceutically acceptable carrier.

21. A method of treating or detecting a disorder, preferably a neurological disorder, in a subject in need thereof, comprising administering to the subject the antibody or antigen-binding fragment of any one of claims 1-5, the conjugate of claim 6, or the fusion construct of any one of claims 7-15, or the pharmaceutical composition of claim 20, preferably, the neurological disorder is selected from the group consisting of neurodegenerative diseases (such as Lewy body disease, postpoliomyelitis syndrome, Shy-Draeger syndrome, olivopontocerebellar atrophy, Parkinson's disease, multiple system atrophy, striatonigral degeneration, spinocerebellar ataxia, spinal muscular atrophy), tauopathies (such as Alzheimer disease and supranuclear palsy), prion diseases (such as bovine spongiform encephalopathy, scrapie, Creutz-feldt-Jakob syndrome, kuru, Gerstmann-Straussler-Scheinker disease, chronic wasting disease, and fatal familial insomnia), bulbar palsy, motor neuron disease, and nervous system heterodegenerative disorders (such as Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (such as Pick's disease, and spinocerebellar ataxia), and cancer of the CNS and/or brain (such as brain metastases resulting from cancer elsewhere in the body).

22. The method of claim 21, wherein the antibody or antigen-binding fragment thereof, the conjugate, or the pharmaceutical composition is administered intravenously.

23. A method of delivering a therapeutic or diagnostic agent across the blood-brain barrier (BBB) of a subject in need thereof, comprising administering to the subject a complex comprising the therapeutic or diagnostic agent coupled to, preferably covalently conjugated to, the antibody or antigen-binding fragment thereof of any one of claims 1 to 5.

24. The method of any one of claims 21 to 24, wherein the administration reduces Fc-mediated effector function and/or does not induce rapid reticulocyte depletion.

25. A method of inducing antibody dependent phagocytosis (ADP) without stimulating secretion of a pro-inflammatory cytokine in a subject in need thereof, comprising administering to the subject a complex comprising a therapeutic antibody or antigen binding fragment thereof coupled to, preferably covalently conjugated to, the antigen-binding fragment thereof of any one of claims 1 to 5, wherein the therapeutic antibody or antigen binding fragment thereof comprises one or more amino acid modifications at positions L234, L235, D270, N297, E318, K320, K322, P331, and P329, such as one, two or three mutations of L234A, L235A and P331S, wherein the numbering of amino acid residues is according to the EU index as set forth in Kabat.

26. The method of claim 25, wherein the therapeutic antibody or antigen binding fragment thereof binds specifically to tau aggregates.


SEQUENCE LISTING (221 sequences)

SEQ ID NO: 1 - protein, 431 aa, Homo sapiens
HHHHHHRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 2 - protein, 431 aa, Macaca fascicularis
HHHHHHRAPRCRELPAQKWWHTGALYRIGDLQAFQGHGSGNLAGLKGRLDYLSSLKVKGLVLGPLHKNQKDDVAQTDLLQIDPNFGSKEDFDNLLQSAKKKSIRVILDLTPNYRGENLWFSTQVDSVATKVKDALEFWLQAGVDGFQVRDIENLKDASSFLAEWENITKGFSEDRLLIAGTNSSDLQQIVSLLESNKDLLLTSSYLSDSSFTGEHTKSLVTQYLNATGNRWCSWSLSQAGLLTSFLPAQLLRLYQLMLFTLPGTPVFSYGDEIGLKAAALPGQPVEAPVMLWDESSFPDIPGAVSANMTVKGQSEDPGSLLSLFRQLSDQRSKERSLLHGDFHTFSSGPGLFSYIRHWDQNERFLVVLNFGDVGLSAGLQASDLPASASLPTKADLVLSTQPGREEGSPLELERLKLEPHEGLLLRFPYVA

SEQ ID NO: 3 - protein, 431 aa, Callithrix jacchus
HHHHHHRAPRCRDLPVQKWWHTGALYRIGDLQGFQGNGAGNLAGLKGHLDYLSSLKVKGLVLGPIHKNQKDDVAGTDLLQIDPNFGSKEDFASLLQSAKKKSIRIILDLTPNYQGENSWFSTQVDTVATKVKDALEFWLQAGVDGFQVRDIENLKDASSFLAEWQNITKGFSEDRLLIAGTNSSDLQQILSLLESNKDLLLTSSYLSNSSFTGEHTKSLVTQYLNATGNRWCSWSLSQAGLLTSFLPAQLLRLYQLMLFTLPGTPVFSYGDEIGLEAAAVPGQPVEAPVMLWDESSFPFISAAGSANMTVKGQSEDPGSLLSLFRRLSDQRSKERSLLHGDFHALSSGPGFFSYIRHWDQNERFLVVLNFGDVGLSAGLQASDLPASASLPAKADLLLSTQPGRDEGSPLELERLKLEPHEGLLLRFPYVA

SEQ ID NO: 4 - protein, 434 aa, Mus musculus
HHHHHHRAPRCRELPVQRWWHKGALYRIGDLQAFVGRDAGGIAGLKSHLEYLSTLKVKGLVLGPIHKNQKDEINETDLKQINPTLGSQEDFKDLLQSAKKKSIHIILDLTPNYQGQNAWFLPAQADIVATKMKEALSSWLQDGVDGFQFRDVGKLMNAPLYLAEWQNITKNLSEDRLLIAGTESSDLQQIVNILESTSDLLLTSSYLSNSTFTGERTESLVTRFLNATGSQWCSWSVSQAGLLADFIPDHLLRLYQLLLFTLPGTPVFSYGDELGLQGALPGQPAKAPLMPWNESSIFHIPRPVSLNMTVKGQNEDPGSLLTQFRRLSDLRGKERSLLHGDFHALSSSPDLFSYIRHWDQNERYLVVLNFRDSGRSARLGASNLPAGISLPASAKLLLSTDSARQSREEDTSLKLENLSLNPYEGLLLQFPFVA

SEQ ID NO: 5 - protein, 435 aa, Rattus rattus
HHHHHHRAPRCRELPVQRWWHKGALYRIGDLQAFVGPEARGIAGLKNHLEYLSTLKVKGLVLGPIHKNQKDEVNETDLKQIDPDLGSQEDFKDLLQSAKKKSIHIILDLTPNYKGQNAWFLPPQADIVATKMKEALSSWLQDGVDGFQVRDVGKLANASLYLAEWQNITKNFSEDRLLIAGTASSDLQQIVNILESTSDLLLTSSYLSQPVFTGEHAELLVIKYLNATGSRWCSWSVSQAGLLTSFIPAQFLRLYQLLLFTLPGTPVFSYGDELGLQAVALPGQPMEAPFMLWNESSNSQTSSPVSLNMTVKGQNEDPGSLLTQFRRLSDLRGKERSLLHGDFDALSSSSGLFSYVRHWDQNERYLVVLNFQDVGLSARVGASNLPAGISLPASANLLLSTDSTRLSREEGTSLSLENLSLNPYEGLLLQFPFVA

SEQ ID NO: 6 - protein, 247 aa, Artificial Sequence
  C98hc 1
QVQLQESGPGLVKPSQTLSLTCTVSGGSISSGAYYWSWIRQHPGKGLEWIGYIYYSGSTYYNPSLKSRVTISLDTSKNQFSLKLSSVTAADTAVYYCARAGPKWELWFDYWGQGTLVTVSSGTEGKSSGSGSESKSTQSALTQPASVSGSPGQSITISCTGTSSDVDNYNYVSWYLQHPGKAPKLMIYDVSNRPSGISTRFSGSKSGNTASLTISGLQAEDEADYYCSSFTSSSTWVFGGGTKLTVL

SEQ ID NO: 7 - protein, 11 aa, Artificial Sequence
  C98hc 1 HCDR1
GGSISSGAYYW

SEQ ID NO: 8 - protein, 7 aa, Artificial Sequence
  C98hc 1 HCDR2
IYYSGST

SEQ ID NO: 9 - protein, 13 aa, Artificial Sequence
  C98hc 1 HCDR3
ARAGPKWELWFDY

SEQ ID NO: 10 - protein, 9 aa, Artificial Sequence
  C98hc 1 LCDR1
SSDVDNYNY

SEQ ID NO: 11 - protein, 7 aa, Artificial Sequence
  C98hc 1 LCDR2
DVSNRPS

SEQ ID NO: 12 - protein, 10 aa, Artificial Sequence
  C98hc 1 LCDR3
SSFTSSSTWV

SEQ ID NO: 13 - protein, 702 aa, Artificial Sequence
  BBBB404 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLQESGPGLVKPSQTLSLTCTVSGGSISSGAYYWSWIRQHPGKGLEWIGYIYYSGSTYYNPSLKSRVTISLDTSKNQFSLKLSSVTAADTAVYYCARAGPKWELWFDYWGQGTLVTVSSGTEGKSSGSGSESKSTQSALTQPASVSGSPGQSITISCTGTSSDVDNYNYVSWYLQHPGKAPKLMIYDVSNRPSGISTRFSGSKSGNTASLTISGLQAEDEADYYCSSFTSSSTWVFGGGTKLTVL

SEQ ID NO: 14 - protein, 218 aa, Artificial Sequence
  BBBB404 LC
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 449 aa, Artificial Sequence
  BBBB404 HC2
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 16 - protein, 700 aa, Artificial Sequence
  BBBB1058 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLQESGPGLVKPSQTLSLTCTVSGGSISSGAYYWSWIRQHPGKGLEWIGYIYYSGSTYYNPSLKSRVTISLDTSKNQFSLKLSSVTAADTAVYYCARAGPKWELWFDYWGQGTLVTVSSGTEGKSSGSGSESKSTQSALTQPASVSGSPGQSITISCTGTSSDVDNYNYVSWYLQHPGKAPKLMIYDVSNRPSGISTRFSGSKSGNTASLTISGLQAEDEADYYCSSFTSSSTWVFGGGTKLTVL

SEQ ID NO: 17 - protein, 214 aa, Artificial Sequence
  BBBB1058 LC
DIQMTQSPSSLSASVGDRVTITCKASQDINRYLNWFQQKPGKAPKTLIYRANRLLDGVPSRFSGSGSGTDFTLTISSLQPEDFATYYCQQYDEFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 447 aa, Artificial Sequence
  BBBB1058 HC2
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 19 - protein, 700 aa, Artificial Sequence
  BBBB1056 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLQESGPGLVKPSQTLSLTCTVSGGSISSGAYYWSWIRQHPGKGLEWIGYIYYSGSTYYNPSLKSRVTISLDTSKNQFSLKLSSVTAADTAVYYCARAGPKWELWFDYWGQGTLVTVSSGTEGKSSGSGSESKSTQSALTQPASVSGSPGQSITISCTGTSSDVDNYNYVSWYLQHPGKAPKLMIYDVSNRPSGISTRFSGSKSGNTASLTISGLQAEDEADYYCSSFTSSSTWVFGGGTKLTVL

SEQ ID NO: 20 - protein, 214 aa, Artificial Sequence; identical to SEQ ID NO: 17
  BBBB1056 LC

SEQ ID NO: 21 - protein, 447 aa, Artificial Sequence
  BBBB1056 HC2
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 22 - protein, 698 aa, Artificial Sequence
  BBBB1097 HC1
QVQLVQSGAEVKKPGASVKVSCKASGHVFTSYDMYWVRQAPGQGLEWIGYIDSDSGDTSYNQKFKGRVTLTVDTSTSTVYMELSSLRSEDTAVYYCAYYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLQESGPGLVKPSQTLSLTCTVSGGSISSGAYYWSWIRQHPGKGLEWIGYIYYSGSTYYNPSLKSRVTISLDTSKNQFSLKLSSVTAADTAVYYCARAGPKWELWFDYWGQGTLVTVSSGTEGKSSGSGSESKSTQSALTQPASVSGSPGQSITISCTGTSSDVDNYNYVSWYLQHPGKAPKLMIYDVSNRPSGISTRFSGSKSGNTASLTISGLQAEDEADYYCSSFTSSSTWVFGGGTKLTVL

SEQ ID NO: 23 - protein, 219 aa, Artificial Sequence
  BBBB1097 LC
DVVMTQSPLSLPVTLGQPASISCKSSQSLLDSRAKTYLTWLQQRPGQSPRRLIYLVSKLDSGVPDRFSGSGSGTDFTLKISRVEAEDVGVYYCWQGTHFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 445 aa, Artificial Sequence
  BBBB1097 HC2
QVQLVQSGAEVKKPGASVKVSCKASGHVFTSYDMYWVRQAPGQGLEWIGYIDSDSGDTSYNQKFKGRVTLTVDTSTSTVYMELSSLRSEDTAVYYCAYYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 25 - protein, 446 aa, Artificial Sequence
  BBBB1226 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVYPPSQEEMTKNQVSLTCLVKGFYPSDIAVEWESNGQPENNYKTTPPVLDSDGSFALVSRLTVDKSRWQEGNVFSCSVMHEALHNHYTQKSLSLSLG

SEQ ID NO: 26 - protein, 218 aa, Artificial Sequence; identical to SEQ ID NO: 14
  BBBB1226 LC

SEQ ID NO: 27 - protein, 699 aa, Artificial Sequence
  BBBB1226 HC2
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVLPPSQEEMTKNQVSLLCLVKGFYPSDIAVEWESNGQPENNYLTWPPVLDSDGSFFLYSRLTVDKSRWQEGNVFSCSVMHEALHNHYTQKSLSLSLGGGSGGSQVQLQESGPGLVKPSQTLSLTCTVSGGSISSGAYYWSWIRQHPGKGLEWIGYIYYSGSTYYNPSLKSRVTISLDTSKNQFSLKLSSVTAADTAVYYCARAGPKWELWFDYWGQGTLVTVSSGTEGKSSGSGSESKSTQSALTQPASVSGSPGQSITISCTGTSSDVDNYNYVSWYLQHPGKAPKLMIYDVSNRPSGISTRFSGSKSGNTASLTISGLQAEDEADYYCSSFTSSSTWVFGGGTKLTVL

SEQ ID NO: 28 - protein, 125 aa, Artificial Sequence
  CD98hc 2
QVQLVESGGGLVQAGGSLRLSCAASGRAFSSNGMGWFRQAPGKEREFVATISCSGTSTYYADSVKGRFTISRDNAKNTVYLQMNSLKPEDTAVYFCAADTRHCMGYTKDEGYAYWGQGTQVTVSS

SEQ ID NO: 29 - protein, 8 aa, Artificial Sequence
  CD98hc 2 HCDR1
GRAFSSNG

SEQ ID NO: 30 - protein, 8 aa, Artificial Sequence
  CD98hc 2 HCDR2
ISCSGTST

SEQ ID NO: 31 - protein, 18 aa, Artificial Sequence
  CD98hc 2 HCDR3
AADTRHCMGYTKDEGYAY

SEQ ID NO: 32 - protein, 580 aa, Artificial Sequence
  BBBB448 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLVESGGGLVQAGGSLRLSCAASGRAFSSNGMGWFRQAPGKEREFVATISCSGTSTYYADSVKGRFTISRDNAKNTVYLQMNSLKPEDTAVYFCAADTRHCMGYTKDEGYAYWGQGTQVTVSS

SEQ ID NO: 33 - protein, 218 aa, Artificial Sequence; identical to SEQ ID NO: 14
  BBBB448 LC

SEQ ID NO: 34 - protein, 449 aa, Artificial Sequence; identical to SEQ ID NO: 15
  BBBB448 HC2

SEQ ID NO: 35 - protein, 578 aa, Artificial Sequence
  BBBB1061 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLVESGGGLVQAGGSLRLSCAASGRAFSSNGMGWFRQAPGKEREFVATISCSGTSTYYADSVKGRFTISRDNAKNTVYLQMNSLKPEDTAVYFCAADTRHCMGYTKDEGYAYWGQGTQVTVSS

SEQ ID NO: 36 - protein, 214 aa, Artificial Sequence; identical to SEQ ID NO: 17
  BBBB1061 LC

SEQ ID NO: 37 - protein, 447 aa, Artificial Sequence; identical to SEQ ID NO: 18
  BBBB1061 HC2

SEQ ID NO: 38 - protein, 578 aa, Artificial Sequence
  BBBB1059 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLVESGGGLVQAGGSLRLSCAASGRAFSSNGMGWFRQAPGKEREFVATISCSGTSTYYADSVKGRFTISRDNAKNTVYLQMNSLKPEDTAVYFCAADTRHCMGYTKDEGYAYWGQGTQVTVSS

SEQ ID NO: 39 - protein, 214 aa, Artificial Sequence; identical to SEQ ID NO: 17
  BBBB1059 LC

SEQ ID NO: 40 - protein, 447 aa, Artificial Sequence; identical to SEQ ID NO: 21
  BBBB1059 HC2

SEQ ID NO: 41 - protein, 576 aa, Artificial Sequence
  BBBB1100 HC1
QVQLVQSGAEVKKPGASVKVSCKASGHVFTSYDMYWVRQAPGQGLEWIGYIDSDSGDTSYNQKFKGRVTLTVDTSTSTVYMELSSLRSEDTAVYYCAYYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLVESGGGLVQAGGSLRLSCAASGRAFSSNGMGWFRQAPGKEREFVATISCSGTSTYYADSVKGRFTISRDNAKNTVYLQMNSLKPEDTAVYFCAADTRHCMGYTKDEGYAYWGQGTQVTVSS

SEQ ID NO: 42 - protein, 219 aa, Artificial Sequence; identical to SEQ ID NO: 23
  BBBB1100 LC

SEQ ID NO: 43 - protein, 445 aa, Artificial Sequence; identical to SEQ ID NO: 24
  BBBB1100 HC2

SEQ ID NO: 44 - protein, 446 aa, Artificial Sequence; identical to SEQ ID NO: 25
  BBBB1228 HC1

SEQ ID NO: 45 - protein, 218 aa, Artificial Sequence; identical to SEQ ID NO: 14
  BBBB1228 LC

SEQ ID NO: 46 - protein, 577 aa, Artificial Sequence
  BBBB1228 HC2
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVLPPSQEEMTKNQVSLLCLVKGFYPSDIAVEWESNGQPENNYLTWPPVLDSDGSFFLYSRLTVDKSRWQEGNVFSCSVMHEALHNHYTQKSLSLSLGGGSGGSQVQLVESGGGLVQAGGSLRLSCAASGRAFSSNGMGWFRQAPGKEREFVATISCSGTSTYYADSVKGRFTISRDNAKNTVYLQMNSLKPEDTAVYFCAADTRHCMGYTKDEGYAYWGQGTQVTVSS

SEQ ID NO: 47 - protein, 125 aa, Artificial Sequence
  CD98hc 3
QVHLVESGGGLVQAGGSLRLSCAASGRTISTYGMGWFRQAPGKEREFVGTISCSGTSTYYTDSVKGRFTISRDNAKNTVYLEMNSLKPEDTAVYFCAADTRHCMGYTKHGGYDYWGQGTQVTVSS

SEQ ID NO: 48 - protein, 8 aa, Artificial Sequence
  CD98hc 3 HCDR1
GRTISTYG

SEQ ID NO: 49 - protein, 7 aa, Artificial Sequence
  CD98hc 3 HCDR2
SCSGTST

SEQ ID NO: 50 - protein, 18 aa, Artificial Sequence
  CD98hc 3 HCDR3
AADTRHCMGYTKHGGYDY

SEQ ID NO: 51 - protein, 580 aa, Artificial Sequence
  BBBB449 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVHLVESGGGLVQAGGSLRLSCAASGRTISTYGMGWFRQAPGKEREFVGTISCSGTSTYYTDSVKGRFTISRDNAKNTVYLEMNSLKPEDTAVYFCAADTRHCMGYTKHGGYDYWGQGTQVTVSS

SEQ ID NO: 52 - protein, 218 aa, Artificial Sequence; identical to SEQ ID NO: 14
  BBBB449 LC

SEQ ID NO: 53 - protein, 449 aa, Artificial Sequence; identical to SEQ ID NO: 15
  BBBB449 HC2

SEQ ID NO: 54 - protein, 578 aa, Artificial Sequence
  BBBB1064 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVHLVESGGGLVQAGGSLRLSCAASGRTISTYGMGWFRQAPGKEREFVGTISCSGTSTYYTDSVKGRFTISRDNAKNTVYLEMNSLKPEDTAVYFCAADTRHCMGYTKHGGYDYWGQGTQVTVSS

SEQ ID NO: 55 - protein, 214 aa, Artificial Sequence; identical to SEQ ID NO: 17
  BBBB1064 LC

SEQ ID NO: 56 - protein, 447 aa, Artificial Sequence; identical to SEQ ID NO: 18
  BBBB1064 HC2

SEQ ID NO: 57 - protein, 578 aa, Artificial Sequence
  BBBB1062 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVHLVESGGGLVQAGGSLRLSCAASGRTISTYGMGWFRQAPGKEREFVGTISCSGTSTYYTDSVKGRFTISRDNAKNTVYLEMNSLKPEDTAVYFCAADTRHCMGYTKHGGYDYWGQGTQVTVSS

SEQ ID NO: 58 - protein, 214 aa, Artificial Sequence; identical to SEQ ID NO: 17
  BBBB1062 LC

SEQ ID NO: 59 - protein, 447 aa, Artificial Sequence; identical to SEQ ID NO: 21
  BBBB1062 HC2

SEQ ID NO: 60 - protein, 576 aa, Artificial Sequence
  BBBB1103 HC1
QVQLVQSGAEVKKPGASVKVSCKASGHVFTSYDMYWVRQAPGQGLEWIGYIDSDSGDTSYNQKFKGRVTLTVDTSTSTVYMELSSLRSEDTAVYYCAYYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVHLVESGGGLVQAGGSLRLSCAASGRTISTYGMGWFRQAPGKEREFVGTISCSGTSTYYTDSVKGRFTISRDNAKNTVYLEMNSLKPEDTAVYFCAADTRHCMGYTKHGGYDYWGQGTQVTVSS

SEQ ID NO: 61 - protein, 219 aa, Artificial Sequence; identical to SEQ ID NO: 23
  BBBB1103 LC

SEQ ID NO: 62 - protein, 445 aa, Artificial Sequence; identical to SEQ ID NO: 24
  BBBB1103 HC2

SEQ ID NO: 63 - protein, 446 aa, Artificial Sequence; identical to SEQ ID NO: 25
  BBBB1229 HC1

SEQ ID NO: 64 - protein, 218 aa, Artificial Sequence; identical to SEQ ID NO: 14
  BBBB1229 LC

SEQ ID NO: 65 - protein, 577 aa, Artificial Sequence
  BBBB1229 HC2
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVLPPSQEEMTKNQVSLLCLVKGFYPSDIAVEWESNGQPENNYLTWPPVLDSDGSFFLYSRLTVDKSRWQEGNVFSCSVMHEALHNHYTQKSLSLSLGGGSGGSQVHLVESGGGLVQAGGSLRLSCAASGRTISTYGMGWFRQAPGKEREFVGTISCSGTSTYYTDSVKGRFTISRDNAKNTVYLEMNSLKPEDTAVYFCAADTRHCMGYTKHGGYDYWGQGTQVTVSS

SEQ ID NO: 66 - protein, 247 aa, Artificial Sequence
  CD98hc 4
QVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSSFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIK

SEQ ID NO: 67 - protein, 8 aa, Artificial Sequence
  CD98hc 4 HCDR1
GFTFSSYG

SEQ ID NO: 68 - protein, 8 aa, Artificial Sequence
  CD98hc 4 HCDR2
ISYDGSNK

SEQ ID NO: 69 - protein, 11 aa, Artificial Sequence
  CD98hc 4 HCDR3
ARAPSSFYFDY

SEQ ID NO: 70 - protein, 12 aa, Artificial Sequence
  CD98hc 4 LCDR1
QSVLFSSNNKNY

SEQ ID NO: 71 - protein, 3 aa, Artificial Sequence
  CD98hc 4 LCDR2
WAS

SEQ ID NO: 72 - protein, 9 aa, Artificial Sequence
  CD98hc 4 LCDR3
QQYYSTPPT

SEQ ID NO: 73 - protein, 702 aa, Artificial Sequence
  BBBB578 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSSFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIK

SEQ ID NO: 74 - protein, 218 aa, Artificial Sequence; identical to SEQ ID NO: 14
  BBBB578 LC

SEQ ID NO: 75 - protein, 449 aa, Artificial Sequence; identical to SEQ ID NO: 15
  BBBB578 HC2

SEQ ID NO: 76 - protein, 700 aa, Artificial Sequence
  BBBB1067 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSSFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIK

SEQ ID NO: 77 - protein, 214 aa, Artificial Sequence; identical to SEQ ID NO: 17
  BBBB1067 LC

SEQ ID NO: 78 - protein, 447 aa, Artificial Sequence; identical to SEQ ID NO: 18
  BBBB1067 HC2

SEQ ID NO: 79 - protein, 700 aa, Artificial Sequence
  BBBB1065 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSSFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIK

SEQ ID NO: 80 - protein, 214 aa, Artificial Sequence; identical to SEQ ID NO: 17
  BBBB1065 LC

SEQ ID NO: 81 - protein, 447 aa, Artificial Sequence; identical to SEQ ID NO: 21
  BBBB1065 HC2

SEQ ID NO: 82 - protein, 698 aa, Artificial Sequence
  BBBB1106 HC1
QVQLVQSGAEVKKPGASVKVSCKASGHVFTSYDMYWVRQAPGQGLEWIGYIDSDSGDTSYNQKFKGRVTLTVDTSTSTVYMELSSLRSEDTAVYYCAYYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSSFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIK

SEQ ID NO: 83 - protein, 219 aa, Artificial Sequence; identical to SEQ ID NO: 23
  BBBB1106 LC

SEQ ID NO: 84 - protein, 445 aa, Artificial Sequence; identical to SEQ ID NO: 24
  BBBB1106 HC2

SEQ ID NO: 85 - protein, 446 aa, Artificial Sequence; identical to SEQ ID NO: 25
  BBBB1230 HC1

SEQ ID NO: 86 - protein, 218 aa, Artificial Sequence; identical to SEQ ID NO: 14
  BBBB1230 LC

SEQ ID NO: 87 - protein, 699 aa, Artificial Sequence
  BBBB1230 HC2
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVLPPSQEEMTKNQVSLLCLVKGFYPSDIAVEWESNGQPENNYLTWPPVLDSDGSFFLYSRLTVDKSRWQEGNVFSCSVMHEALHNHYTQKSLSLSLGGGSGGSQVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSSFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIK

SEQ ID NO: 88 - protein, 238 aa, Artificial Sequence
  CD98hc 5
QVQLQESGPGLVKPSETLSLTCTVSGGSISSSYWSWIRQPPGKGLEWIGYIYNSGSTNYNPSLKSRVTISVDTSKNQFSLKLSSVTAADTAVYYCARETYYFDYWGQGTLVTVSSGTEGKSSGSGSESKSTDIVMTQSQKFMSTSVGDRVSVTCKASQNVGINVAWYQKKPGQPPKALIYSAAYRYSGVPDRFTGSGSGTDFTLTISNVQSEDLAEYFCQQYNSYPLTFGSGTKLEIK

SEQ ID NO: 89 - protein, 8 aa, Artificial Sequence
  CD98hc 5 HCDR1
GGSISSSY

SEQ ID NO: 90 - protein, 7 aa, Artificial Sequence
  CD98hc 5 HCDR2
IYNSGST

SEQ ID NO: 91 - protein, 9 aa, Artificial Sequence
  CD98hc 5 HCDR3
ARETYYFDY

SEQ ID NO: 92 - protein, 6 aa, Artificial Sequence
  CD98hc 5 LCDR1
QNVGIN

SEQ ID NO: 93 - protein, 3 aa, Artificial Sequence
  CD98hc 5 LCDR2
SAA

SEQ ID NO: 94 - protein, 9 aa, Artificial Sequence
  CD98hc 5 LCDR3
QQYNSYPLT

SEQ ID NO: 95 - protein, 693 aa, Artificial Sequence
  BBBB584 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLQESGPGLVKPSETLSLTCTVSGGSISSSYWSWIRQPPGKGLEWIGYIYNSGSTNYNPSLKSRVTISVDTSKNQFSLKLSSVTAADTAVYYCARETYYFDYWGQGTLVTVSSGTEGKSSGSGSESKSTDIVMTQSQKFMSTSVGDRVSVTCKASQNVGINVAWYQKKPGQPPKALIYSAAYRYSGVPDRFTGSGSGTDFTLTISNVQSEDLAEYFCQQYNSYPLTFGSGTKLEIK

SEQ ID NO: 96 - protein, 218 aa, Artificial Sequence; identical to SEQ ID NO: 14
  BBBB584 LC

SEQ ID NO: 97 - protein, 449 aa, Artificial Sequence; identical to SEQ ID NO: 15
  BBBB584 HC2

SEQ ID NO: 98 - protein, 691 aa, Artificial Sequence
  BBBB1070 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLQESGPGLVKPSETLSLTCTVSGGSISSSYWSWIRQPPGKGLEWIGYIYNSGSTNYNPSLKSRVTISVDTSKNQFSLKLSSVTAADTAVYYCARETYYFDYWGQGTLVTVSSGTEGKSSGSGSESKSTDIVMTQSQKFMSTSVGDRVSVTCKASQNVGINVAWYQKKPGQPPKALIYSAAYRYSGVPDRFTGSGSGTDFTLTISNVQSEDLAEYFCQQYNSYPLTFGSGTKLEIK

SEQ ID NO: 99 - protein, 214 aa, Artificial Sequence; identical to SEQ ID NO: 17
  BBBB1070 LC

SEQ ID NO: 100 - protein, 447 aa, Artificial Sequence; identical to SEQ ID NO: 18
  BBBB1070 HC2

SEQ ID NO: 101 - protein, 691 aa, Artificial Sequence
  BBBB1068 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLQESGPGLVKPSETLSLTCTVSGGSISSSYWSWIRQPPGKGLEWIGYIYNSGSTNYNPSLKSRVTISVDTSKNQFSLKLSSVTAADTAVYYCARETYYFDYWGQGTLVTVSSGTEGKSSGSGSESKSTDIVMTQSQKFMSTSVGDRVSVTCKASQNVGINVAWYQKKPGQPPKALIYSAAYRYSGVPDRFTGSGSGTDFTLTISNVQSEDLAEYFCQQYNSYPLTFGSGTKLEIK

SEQ ID NO: 102 - protein, 214 aa, Artificial Sequence; identical to SEQ ID NO: 17
  BBBB1068 LC

SEQ ID NO: 103 - protein, 447 aa, Artificial Sequence; identical to SEQ ID NO: 21
  BBBB1068 HC2

SEQ ID NO: 104 - protein, 689 aa, Artificial Sequence
  BBBB1109 HC1
QVQLVQSGAEVKKPGASVKVSCKASGHVFTSYDMYWVRQAPGQGLEWIGYIDSDSGDTSYNQKFKGRVTLTVDTSTSTVYMELSSLRSEDTAVYYCAYYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLQESGPGLVKPSETLSLTCTVSGGSISSSYWSWIRQPPGKGLEWIGYIYNSGSTNYNPSLKSRVTISVDTSKNQFSLKLSSVTAADTAVYYCARETYYFDYWGQGTLVTVSSGTEGKSSGSGSESKSTDIVMTQSQKFMSTSVGDRVSVTCKASQNVGINVAWYQKKPGQPPKALIYSAAYRYSGVPDRFTGSGSGTDFTLTISNVQSEDLAEYFCQQYNSYPLTFGSGTKLEIK

SEQ ID NO: 105 - protein, 219 aa, Artificial Sequence; identical to SEQ ID NO: 23
  BBBB1109 LC

SEQ ID NO: 106 - protein, 445 aa, Artificial Sequence; identical to SEQ ID NO: 24
  BBBB1109 HC2

SEQ ID NO: 107 - protein, 446 aa, Artificial Sequence; identical to SEQ ID NO: 25
  BBBB1231 HC1

SEQ ID NO: 108 - protein, 218 aa, Artificial Sequence; identical to SEQ ID NO: 14
  BBBB1231 LC

SEQ ID NO: 109 - protein, 690 aa, Artificial Sequence
  BBBB1231 HC2
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVLPPSQEEMTKNQVSLLCLVKGFYPSDIAVEWESNGQPENNYLTWPPVLDSDGSFFLYSRLTVDKSRWQEGNVFSCSVMHEALHNHYTQKSLSLSLGGGSGGSQVQLQESGPGLVKPSETLSLTCTVSGGSISSSYWSWIRQPPGKGLEWIGYIYNSGSTNYNPSLKSRVTISVDTSKNQFSLKLSSVTAADTAVYYCARETYYFDYWGQGTLVTVSSGTEGKSSGSGSESKSTDIVMTQSQKFMSTSVGDRVSVTCKASQNVGINVAWYQKKPGQPPKALIYSAAYRYSGVPDRFTGSGSGTDFTLTISNVQSEDLAEYFCQQYNSYPLTFGSGTKLEIK

SEQ ID NO: 110 - protein, 448 aa, Artificial Sequence
  PT1B844 HC
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 214 aa, Artificial Sequence; identical to SEQ ID NO: 17
  PT1B844 LC

SEQ ID NO: 112 - protein, 251 aa, Artificial Sequence
  CD98hc 6
QVQLVQSGAEVKKPGASVKVSCKASGYTFTSYYIHWVRQAPGQGLEWMGLINPSGGTTNYPQKFQGRVTMTRDTSTSTVYMELSSLRSEDTAVYYCARDPNDYGGPKYFPHWGQGTLVTVSSGTEGKSSGSGSESKSTDIVMTQSPDSLAVSLGERATINCRSSQSVLYRSNNKNYLGWYQQKPGQPPKLLIYWASTRESGVPDRFSGSGSGTDFTLTISSLQAEDVAVYYCHQYYSTPLTFGGGTKVDIK

SEQ ID NO: 113 - protein, 8 aa, Artificial Sequence
  CD98hc 6 HCDR1
GYTFTSYY

SEQ ID NO: 114 - protein, 8 aa, Artificial Sequence
  CD98hc 6 HCDR2
INPSGGTT

SEQ ID NO: 115 - protein, 15 aa, Artificial Sequence
  CD98hc 6 HCDR3
ARDPNDYGGPKYFPH

SEQ ID NO: 116 - protein, 12 aa, Artificial Sequence
  CD98hc 6 LCDR1
QSVLYRSNNKNY

SEQ ID NO: 117 - protein, 3 aa, Artificial Sequence
  CD98hc 6 LCDR2
WAS

SEQ ID NO: 118 - protein, 9 aa, Artificial Sequence
  CD98hc 6 LCDR3
HQYYSTPLT

SEQ ID NO: 119 - protein, 706 aa, Artificial Sequence
  BBBB580 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLVQSGAEVKKPGASVKVSCKASGYTFTSYYIHWVRQAPGQGLEWMGLINPSGGTTNYPQKFQGRVTMTRDTSTSTVYMELSSLRSEDTAVYYCARDPNDYGGPKYFPHWGQGTLVTVSSGTEGKSSGSGSESKSTDIVMTQSPDSLAVSLGERATINCRSSQSVLYRSNNKNYLGWYQQKPGQPPKLLIYWASTRESGVPDRFSGSGSGTDFTLTISSLQAEDVAVYYCHQYYSTPLTFGGGTKVDIK

SEQ ID NO: 120 - protein, 218 aa, Artificial Sequence; identical to SEQ ID NO: 14
  BBBB580 LC

SEQ ID NO: 121 - protein, 449 aa, Artificial Sequence; identical to SEQ ID NO: 15
  BBBB580 HC2

SEQ ID NO: 122 - protein, 253 aa, Artificial Sequence
  CD98hc7
QVQLVQSGAEVKKPGASVRVSCKASEYTFTSYYMHWIRQAPGQGLEWMGLINPSGGSTSYAQKFQGRVTMTRDTSTTTVYMELSSLRSEDTALYYCARAGYCTINSCFNWFDPWGQGTLVTVSSGTEGKSSGSGSESKSTDIVMTQSPDSLAESLGERATINCKSSQSVLYASRNKNYLAWYQQRPGQPPKLLIYWASTRESGVPDRFSGSGSGTDFTLTISSLQAEDVAIYYCQQYYSTPFTFGPGTKVDIK

SEQ ID NO: 123 - protein, 8 aa, Artificial Sequence
  CD98hc7 HCDR1
EYTFTSYY

SEQ ID NO: 124 - protein, 8 aa, Artificial Sequence
  CD98hc7 HCDR2
INPSGGST

SEQ ID NO: 125 - protein, 17 aa, Artificial Sequence
  CD98hc7 HCDR3
ARAGYCTINSCFNWFDP

SEQ ID NO: 126 - protein, 12 aa, Artificial Sequence
  CD98hc7 LCDR1
QSVLYASRNKNY

SEQ ID NO: 127 - protein, 3 aa, Artificial Sequence
  CD98hc7 LCDR2
WAS

SEQ ID NO: 128 - protein, 9 aa, Artificial Sequence
  CD98hc7 LCDR3
QQYYSTPFT

SEQ ID NO: 129 - protein, 708 aa, Artificial Sequence
  BBBB586 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLVQSGAEVKKPGASVRVSCKASEYTFTSYYMHWIRQAPGQGLEWMGLINPSGGSTSYAQKFQGRVTMTRDTSTTTVYMELSSLRSEDTALYYCARAGYCTINSCFNWFDPWGQGTLVTVSSGTEGKSSGSGSESKSTDIVMTQSPDSLAESLGERATINCKSSQSVLYASRNKNYLAWYQQRPGQPPKLLIYWASTRESGVPDRFSGSGSGTDFTLTISSLQAEDVAIYYCQQYYSTPFTFGPGTKVDIK

SEQ ID NO: 130 - protein, 218 aa, Artificial Sequence; identical to SEQ ID NO: 14
  BBBB586 LC

SEQ ID NO: 131 - protein, 449 aa, Artificial Sequence; identical to SEQ ID NO: 15
  BBBB586 HC2

SEQ ID NO: 132 - protein, 249 aa, Artificial Sequence
  CD98hc8
QVHLVQSGVEVKKPGASVKVSCKASGYTFTSYYIHWVRQAPGQGLEWMGLFNPSGGSTSYAQKFQGRVTLTRDTSTSTVYMELSSLRSEDTAVYYCARGGVGATGYFDYWGQGTLVTVSSGTEGKSSGSGSESKSTDIVMTQSPDSLAVSLGERATINCKSSQSVLYRSNNKNYLAWYQQKPGQPPKLLIYWASTRESGVPDRFSGSGSETDFTLTISSLQAEDVALYYCQQYYGTPFTFGPGTKVDIK

SEQ ID NO: 133 - protein, 8 aa, Artificial Sequence
  CD98hc8 HCDR1
GYTFTSYY

SEQ ID NO: 134 - protein, 8 aa, Artificial Sequence
  CD98hc8 HCDR2
FNPSGGST

SEQ ID NO: 135 - protein, 13 aa, Artificial Sequence
  CD98hc8 HCDR3
ARGGVGATGYFDY

SEQ ID NO: 136 - protein, 12 aa, Artificial Sequence
  CD98hc8 LCDR1
QSVLYRSNNKNY

SEQ ID NO: 137 - protein, 3 aa, Artificial Sequence
  CD98hc8 LCDR2
WAS

SEQ ID NO: 138 - protein, 9 aa, Artificial Sequence
  CD98hc8 LCDR3
QQYYGTPFT

SEQ ID NO: 139 - protein, 704 aa, Artificial Sequence
  BBBB593 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVHLVQSGVEVKKPGASVKVSCKASGYTFTSYYIHWVRQAPGQGLEWMGLFNPSGGSTSYAQKFQGRVTLTRDTSTSTVYMELSSLRSEDTAVYYCARGGVGATGYFDYWGQGTLVTVSSGTEGKSSGSGSESKSTDIVMTQSPDSLAVSLGERATINCKSSQSVLYRSNNKNYLAWYQQKPGQPPKLLIYWASTRESGVPDRFSGSGSETDFTLTISSLQAEDVALYYCQQYYGTPFTFGPGTKVDIK

SEQ ID NO: 140 - protein, 218 aa, Artificial Sequence; identical to SEQ ID NO: 14
  BBBB593 LC

SEQ ID NO: 141 - protein, 449 aa, Artificial Sequence; identical to SEQ ID NO: 15
  BBBB593 HC2

SEQ ID NO: 142 - protein, 125 aa, Artificial Sequence
  CD98hc9
QVQLVESGGGLVQAGGSLRLSCAASGRTFSRYGMGWFRQAPGKEREFVATISCSGTSTYYADSLKGRFTISRDNAKNTVYLQMNSLKPEDTAVYFCAADTRHCMGYTKDGGYAYWGQGTQVTVSS

SEQ ID NO: 143 - protein, 8 aa, Artificial Sequence
  CD98hc9 HCDR1
GRTFSRYG

SEQ ID NO: 144 - protein, 8 aa, Artificial Sequence
  CD98hc9 HCDR2
ISCSGTST

SEQ ID NO: 145 - protein, 18 aa, Artificial Sequence
  CD98hc9 HCDR3
AADTRHCMGYTKDGGYAY

SEQ ID NO: 146 - protein, 580 aa, Artificial Sequence
  BBBB445 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLVESGGGLVQAGGSLRLSCAASGRTFSRYGMGWFRQAPGKEREFVATISCSGTSTYYADSLKGRFTISRDNAKNTVYLQMNSLKPEDTAVYFCAADTRHCMGYTKDGGYAYWGQGTQVTVSS

SEQ ID NO: 147 - protein, 218 aa, Artificial Sequence; identical to SEQ ID NO: 14
  BBBB445 LC

SEQ ID NO: 148 - protein, 449 aa, Artificial Sequence; identical to SEQ ID NO: 15
  BBBB445 HC2

SEQ ID NO: 149 - protein, 238 aa, Artificial Sequence
  CD98hc10
QVQLVQSGAEVKKPGASVKVSCKASGYTFTGYYLHWVRQAPGQGLEWMGWINPNSGGSNSAQKFQGRVTMTRDTSISTAYMEVSRLTSDDTAVYYCLRGKTFDPWGQGTLVTVSSGTEGKSSGSGSESKSTDIQLTQSPSFLSASVGDRVTITCRASQGISSHLAWYQQKPGKAPKLLIYAASTLQSGVPSRFSGSGSGTEFTLTISSLQPEDFATYYCQQLNNYPLTFGQGTRLEIK

SEQ ID NO: 150 - protein, 8 aa, Artificial Sequence
  CD98hc10 HCDR1
GYTFTGYY

SEQ ID NO: 151 - protein, 8 aa, Artificial Sequence
  CD98hc10 HCDR2
INPNSGGS

SEQ ID NO: 152 - protein, 8 aa, Artificial Sequence
  CD98hc10 HCDR3
LRGKTFDP

SEQ ID NO: 153 - protein, 6 aa, Artificial Sequence
  CD98hc10 LCDR1
QGISSH

SEQ ID NO: 154 - protein, 3 aa, Artificial Sequence
  CD98hc10 LCDR2
AAS

SEQ ID NO: 155 - protein, 9 aa, Artificial Sequence
  CD98hc10 LCDR3
QQLNNYPLT

SEQ ID NO: 156 - protein, 693 aa, Artificial Sequence
  BBBB589 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLVQSGAEVKKPGASVKVSCKASGYTFTGYYLHWVRQAPGQGLEWMGWINPNSGGSNSAQKFQGRVTMTRDTSISTAYMEVSRLTSDDTAVYYCLRGKTFDPWGQGTLVTVSSGTEGKSSGSGSESKSTDIQLTQSPSFLSASVGDRVTITCRASQGISSHLAWYQQKPGKAPKLLIYAASTLQSGVPSRFSGSGSGTEFTLTISSLQPEDFATYYCQQLNNYPLTFGQGTRLEIK

SEQ ID NO: 157 - protein, 218 aa, Artificial Sequence; identical to SEQ ID NO: 14
  BBBB589 LC

SEQ ID NO: 158 - protein, 449 aa, Artificial Sequence; identical to SEQ ID NO: 15
  BBBB589 HC2

SEQ ID NO: 159 - protein, 253 aa, Artificial Sequence
  CD98hc11
QVQLVQSGAEVKKPGASVKVSCKASGYTFTSYYMHWVRQAPGQGLEWMGLINPSGGSTSYAQKFQGRVTMTRDTSTSTVYMELSSLRSEDTAVYYCARAGYCRSTTSCFNWFDPWGQGTLVTVSSGTEGKSSGSGSESKSTDIVMTQSPDSLAVSLGERATINCKSSQSVLYNSNNKNYLAWYQQKPGQPPKLLIYWASTRESGVPDRFSGSGSGTDFTLTFSSLQAEDVAVYYCQQYYSTPFTFGPGTKVDI

SEQ ID NO: 160 - protein, 8 aa, Artificial Sequence
  CD98hc11 HCDR1
GYTFTSYY

SEQ ID NO: 161 - protein, 7 aa, Artificial Sequence
  CD98hc11 HCDR2
NPSGGST

SEQ ID NO: 162 - protein, 18 aa, Artificial Sequence
  CD98hc11 HCDR3
ARAGYCRSTTSCFNWFDP

SEQ ID NO: 163 - protein, 12 aa, Artificial Sequence
  CD98hc11 LCDR1
QSVLYNSNNKNY

SEQ ID NO: 164 - protein, 3 aa, Artificial Sequence
  CD98hc11 LCDR2
WAS

SEQ ID NO: 165 - protein, 9 aa, Artificial Sequence
  CD98hc11 LCDR3
QQYYSTPFT

SEQ ID NO: 166 - protein, 708 aa, Artificial Sequence
  BBBB594 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLVQSGAEVKKPGASVKVSCKASGYTFTSYYMHWVRQAPGQGLEWMGLINPSGGSTSYAQKFQGRVTMTRDTSTSTVYMELSSLRSEDTAVYYCARAGYCRSTTSCFNWFDPWGQGTLVTVSSGTEGKSSGSGSESKSTDIVMTQSPDSLAVSLGERATINCKSSQSVLYNSNNKNYLAWYQQKPGQPPKLLIYWASTRESGVPDRFSGSGSGTDFTLTFSSLQAEDVAVYYCQQYYSTPFTFGPGTKVDI

SEQ ID NO: 167 - protein, 218 aa, Artificial Sequence; identical to SEQ ID NO: 14
  BBBB594 LC

SEQ ID NO: 168 - protein, 449 aa, Artificial Sequence; identical to SEQ ID NO: 15
  BBBB594 HC2

SEQ ID NO: 169 - protein, 246 aa, Artificial Sequence
  CD98hc12
EVQLVESGGGLVQPGGSLRLSCAASGFTLSSSWMHWVRQAPGKGLVWVSRINSDGSSTSYADSVKGRFTISRDNAKNTLYLQMNSLRAEDTALYYCARWNWGWYFDLWGRGTLVTVSSGTEGKSSGSGSESKSTDIVMTQSPLSLPVTPGEPASISCRSSQSLLHSHGDNYLDWFLQKPGQSPQLLIYLGSNRASGVPDRFSGSGSGTDFTLKISRVEAEDVGVYYCMQTLETPYTFGQGTKLQIK

SEQ ID NO: 170 - protein, 8 aa, Artificial Sequence
  CD98hc12 HCDR1
GFTLSSSW

SEQ ID NO: 171 - protein, 8 aa, Artificial Sequence
  CD98hc12 HCDR2
INSDGSST

SEQ ID NO: 172 - protein, 11 aa, Artificial Sequence
  CD98hc12 HCDR3
ARWNWGWYFDL

SEQ ID NO: 173 - protein, 11 aa, Artificial Sequence
  CD98hc12 LCDR1
QSLLHSHGDNY

SEQ ID NO: 174 - protein, 3 aa, Artificial Sequence
  CD98hc12 LCDR2
LGS

SEQ ID NO: 175 - protein, 9 aa, Artificial Sequence
  CD98hc12 LCDR3
MQTLETPYT

SEQ ID NO: 176 - protein, 701 aa, Artificial Sequence
  BBBB602 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLVESGGGLVQPGGSLRLSCAASGFTLSSSWMHWVRQAPGKGLVWVSRINSDGSSTSYADSVKGRFTISRDNAKNTLYLQMNSLRAEDTALYYCARWNWGWYFDLWGRGTLVTVSSGTEGKSSGSGSESKSTDIVMTQSPLSLPVTPGEPASISCRSSQSLLHSHGDNYLDWFLQKPGQSPQLLIYLGSNRASGVPDRFSGSGSGTDFTLKISRVEAEDVGVYYCMQTLETPYTFGQGTKLQIK

SEQ ID NO: 177 - protein, 218 aa, Artificial Sequence; identical to SEQ ID NO: 14
  BBBB602 LC

SEQ ID NO: 178 - protein, 449 aa, Artificial Sequence; identical to SEQ ID NO: 15
  BBBB602 HC2

SEQ ID NO: 179 - protein, 247 aa, Artificial Sequence
  CD98hc13
EVQLVESGGGLVQPGGSLRLSCVASGFTFSSYWMHWVRQAPGKGLVWVSRINSDGSIIRYADSVKGRFTISRDNAKNTLYLQMNSLRAEDTAVYYCVREGRYTGSSLDYWGQGALVTVSSGTEGKSSGSGSESKSTQSVLTQPPSVSGAPGQRVAISCTGNNSNIGAGYDVHWYQQLPGTAPQLLIYGNSDRPSGVPDRFSGSKSGTSASLAITGLQAEDEAAYYCQSYDSSLTGWVFGGGTKLTVL

SEQ ID NO: 180 - protein, 8 aa, Artificial Sequence
  CD98hc13 HCDR1
GFTFSSYW

SEQ ID NO: 181 - protein, 8 aa, Artificial Sequence
  CD98hc13 HCDR2
INSDGSII

SEQ ID NO: 182 - protein, 13 aa, Artificial Sequence
  CD98hc13 HCDR3
VREGRYTGSSLDY

SEQ ID NO: 183 - protein, 9 aa, Artificial Sequence
  CD98hc13 LCDR1
NSNIGAGYD

SEQ ID NO: 184 - protein, 3 aa, Artificial Sequence
  CD98hc13 LCDR2
GNS

SEQ ID NO: 185 - protein, 11 aa, Artificial Sequence
  CD98hc13 LCDR3
QSYDSSLTGWV

SEQ ID NO: 186 - protein, 702 aa, Artificial Sequence
  BBBB610 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLVESGGGLVQPGGSLRLSCVASGFTFSSYWMHWVRQAPGKGLVWVSRINSDGSIIRYADSVKGRFTISRDNAKNTLYLQMNSLRAEDTAVYYCVREGRYTGSSLDYWGQGALVTVSSGTEGKSSGSGSESKSTQSVLTQPPSVSGAPGQRVAISCTGNNSNIGAGYDVHWYQQLPGTAPQLLIYGNSDRPSGVPDRFSGSKSGTSASLAITGLQAEDEAAYYCQSYDSSLTGWVFGGGTKLTVL

SEQ ID NO: 187 - protein, 218 aa, Artificial Sequence; identical to SEQ ID NO: 14
  BBBB610 LC

SEQ ID NO: 188 - protein, 449 aa, Artificial Sequence; identical to SEQ ID NO: 15
  BBBB610 HC2

SEQ ID NO: 189 - protein, 16 aa, Artificial Sequence
  Linker
GTEGKSSGSGSESKST

SEQ ID NO: 190 - protein, 6 aa, Artificial Sequence
  Linker
GGSGGS

SEQ ID NO: 191 - protein, 6 aa, Artificial Sequence
  Linker
GGAGGA

SEQ ID NO: 192 - protein, 20 aa, Artificial Sequence
  Biotinylated phospho-tau oligomer
GTPGSRSRPSLPPPTREPLL

SEQ ID NO: 193 - protein, 247 aa, Artificial Sequence
  CD98hc14
QVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSAFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIK

SEQ ID NO: 194 - protein, 8 aa, Artificial Sequence
  CD98hc14 HCDR1
GFTFSSYG

SEQ ID NO: 195 - protein, 8 aa, Artificial Sequence
  CD98hc14 HCDR2
ISYDGSNK

SEQ ID NO: 196 - protein, 11 aa, Artificial Sequence
  CD98hc14 HCDR3
ARAPSAFYFDY

SEQ ID NO: 197 - protein, 12 aa, Artificial Sequence
  CD98hc14 LCDR1
QSVLFSSNNKNY

SEQ ID NO: 198 - protein, 3 aa, Artificial Sequence
  CD98hc14 LCDR2
WAS

SEQ ID NO: 199 - protein, 9 aa, Artificial Sequence
  CD98hc14 LCDR3
QQYYSTPPT

SEQ ID NO: 200 - protein, 700 aa, Artificial Sequence
  BBBB1201 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSAFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIK

SEQ ID NO: 201 - protein, 214 aa, Artificial Sequence; identical to SEQ ID NO: 17
  BBBB1201 LC

SEQ ID NO: 202 - protein, 447 aa, Artificial Sequence; identical to SEQ ID NO: 18
  BBBB1201 HC2

SEQ ID NO: 203 - protein, 247 aa, Artificial Sequence
  CD98hc15
QVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSHFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIK

SEQ ID NO: 204 - protein, 8 aa, Artificial Sequence
  CD98hc15 HCDR1
GFTFSSYG

SEQ ID NO: 205 - protein, 8 aa, Artificial Sequence
  CD98hc15 HCDR2
ISYDGSNK

SEQ ID NO: 206 - protein, 11 aa, Artificial Sequence
  CD98hc15 HCDR3
ARAPSHFYFDY

SEQ ID NO: 207 - protein, 12 aa, Artificial Sequence
  CD98hc15 LCDR1
QSVLFSSNNKNY

SEQ ID NO: 208 - protein, 3 aa, Artificial Sequence
  CD98hc15 LCDR2
WAS

SEQ ID NO: 209 - protein, 9 aa, Artificial Sequence
  CD98hc15 LCDR3
QQYYSTPPT

SEQ ID NO: 210 - protein, 700 aa, Artificial Sequence
  BBBB1202 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSHFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIK

SEQ ID NO: 211 - protein, 214 aa, Artificial Sequence; identical to SEQ ID NO: 17
  BBBB1202 LC

SEQ ID NO: 212 - protein, 447 aa, Artificial Sequence; identical to SEQ ID NO: 18
  BBBB1202 HC2

SEQ ID NO: 213 - protein, 10 aa, Artificial Sequence
  PT1B844 HCDR1
GFTFSSYAMS

SEQ ID NO: 214 - protein, 9 aa, Artificial Sequence
  PT1B844 HCDR2
SISKGGNTY

SEQ ID NO: 215 - protein, 10 aa, Artificial Sequence
  PT1B844 HCDR3
GWGDYGWFAY

SEQ ID NO: 216 - protein, 11 aa, Artificial Sequence
  PT1B844 LCDR1
KASQDINRYLN

SEQ ID NO: 217 - protein, 7 aa, Artificial Sequence
  PT1B844 LCDR2
RANRLLD

SEQ ID NO: 218 - protein, 9 aa, Artificial Sequence
  PT1B844 LCDR3
QQYDEFPLT

SEQ ID NO: 219 - protein, 446 aa, Artificial Sequence; identical to SEQ ID NO: 25
  BBBB1227 HC1

SEQ ID NO: 220 - protein, 218 aa, Artificial Sequence; identical to SEQ ID NO: 14
  BBBB1227 LC

SEQ ID NO: 221 - protein, 577 aa, Artificial Sequence
  BBBB1227 HC2
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVLPPSQEEMTKNQVSLLCLVKGFYPSDIAVEWESNGQPENNYLTWPPVLDSDGSFFLYSRLTVDKSRWQEGNVFSCSVMHEALHNHYTQKSLSLSLGGGSGGSQVQLVESGGGLVQAGGSLRLSCAASGRTFSRYGMGWFRQAPGKEREFVATISCSGTSTYYADSLKGRFTISRDNAKNTVYLQMNSLKPEDTAVYFCAADTRHCMGYTKDGGYAYWGQGTQVTVSS